(19) United States
(12) Patent Application Publication
(10) Pub. No.: US 2026/0234713 A1
(43) Pub. Date: Aug. 13, 2026

(54) METHODS AND COMPOSITIONS FOR ANALYZING NUCLEIC ACIDS

(71) Applicant: Countable Labs, Inc., Palo Alto, CA (US)

(72) Inventors: Eleen Yee Lam SHUM, San Carlos, CA (US); Hei Mun Christina FAN, Palo Alto, CA (US); Stephen P.A. FODOR, Palo Alto, CA (US); Janice Hoiyi LAI, Mountain View, CA (US); Jung Won KEUM, Palo Alto, CA (US); Haeun Grace LEE, Palo Alto, CA (US); Sixing LI, Palo Alto, CA (US); Emily Jaene-Blair JACOBS, Palo Alto, CA (US)

(73) Assignee: Countable Labs, Inc., Palo Alto, CA (US)

(21) Appl. No.: 19/638,454

(22) Filed: Apr. 3, 2026

Related U.S. Application Data
(63) Continuation of application No. PCT/US24/49654, filed on Oct. 2, 2024.
(60) Provisional application No. 63/588,652, filed on Oct. 6, 2023.

(51) Int. Cl. C12Q 1/686

(52) U.S. Cl. CPC C12Q 1/686

(57) ABSTRACT

Provided herein are methods and compositions for analyzing nucleic acids within partitions loaded with a high occupancy. In some cases, partitions may be provided that include portions of a nucleic acid sample, The partitions may also include processing materials. The portions of the nucleic acid sample may be reacted with the processing materials. In some cases an imaging system may be used to scan the partitions. Signals generated from scanning the partitions may be used to identify a plurality of nucleic acid molecules.

### CROSS-REFERENCE

This application is a continuation of PCT Application No. PCT/US24/49654, filed Oct. 2, 2024, which claims priority to U.S. Provisional Patent Application No. 63/588,652, filed Oct. 6, 2023, which is herein incorporated by reference in their entireties for all purposes.

### BACKGROUND

Nucleic acid molecules can be analyzed using a variety of technologies. Polymerase chain reaction (PCR) and digital PCR (dPCR) have been used to analyze nucleic acid molecules. In some cases, partitions have been used to analyze nucleic acid molecules using dPCR.

### SUMMARY

Aspects disclosed herein provide methods comprising: (a) providing a plurality of partitions comprising at least 1,000 partitions, wherein each partition of the plurality of partitions comprises: (i) a portion of a nucleic acid sample; and (ii) processing materials, wherein a distribution of nucleic acid material of the nucleic acid sample to partition has an average of at least 10 femtograms of the nucleic acid material per partition and wherein each partition comprises an average volume of the plurality of partitions is less than or equal to 10 picolitre (pl); (b) reacting the processing materials of each partition of the plurality of partitions with the portion of the nucleic acid sample of each partition of the plurality of partitions; (c) detecting signals from at least a subset of the plurality of partitions; and (d) using the signals detected in (c) to identify a plurality of nucleic acid molecules of the nucleic acid sample.

In some embodiments, the plurality of partitions comprises at least 10,000 partitions. In some embodiments, the plurality of partitions comprises at least 1,000,000 partitions. In some embodiments, the distribution of nucleic acid material to partition has an average of at least 100 femtograms of the nucleic acid sample per partition. In some embodiments, the nucleic acid material to partition has an average of at least 200 femtograms of the nucleic acid sample per partition. In some embodiments, the average volume of the plurality of partitions is less than or equal to 5 pL. In some embodiments, the average volume of the plurality of partitions is less than or equal to 1 pL.

Other aspects disclosed herein provide methods comprising: (a) providing a plurality of partitions comprising at least 1,000,000 partitions, wherein each partition of the plurality of partitions comprises: (i) a portion of a nucleic acid sample; and (ii) processing materials, wherein a distribution of nucleic acid material of the nucleic acid sample to partition has an average of at least 10 femtograms of the nucleic acid material per partition; (b) reacting the processing materials of each partition of the plurality of partitions with the portion of the nucleic acid sample of each partition of the plurality of partitions; (c) detecting signals from at least a subset of the plurality of partitions; and (d) using the signals detected in (c) to identify a plurality of nucleic acid molecules of the nucleic acid sample.

In some embodiments, the plurality of partitions comprises at least 10,000,000 partitions. In some embodiments, the plurality of partitions comprises at least 20,000,000 partitions. In some embodiments, the distribution of nucleic acid material to partition has an average of at least 100 femtograms of the nucleic acid sample per partition. In some embodiments, the distribution of nucleic acid material to partition has an average of at least 200 femtograms of the nucleic acid sample per partition. In some embodiments, the nucleic acid sample comprises at least 0.1 microgram of nucleic acid material. In some embodiments, the nucleic acid sample comprises at least 0.5 microgram of nucleic acid material. In some embodiments, the nucleic acid sample comprises at least 1.0 microgram of nucleic acid material. In some embodiments, the nucleic acid sample comprises at least 5.0 micrograms of nucleic acid material. In some embodiments, the plurality of partitions are part of a gel matrix. In some embodiments, the plurality of partitions are part of an emulsion. In some embodiments, the nucleic acid sample comprises cell-free nucleic acid molecules. In some embodiments, nucleic acid molecules comprise deoxyribonucleic acid molecules. In some embodiments, the nucleic acid molecules comprise ribonucleic acid molecules. In some embodiments, the processing materials comprise an enzyme. In some embodiments, the enzyme comprises a polymerase. In some embodiments, the processing materials comprise nucleotides. In some embodiments, the processing materials comprise one or more probes that bind to one or more nucleic acid molecules of the plurality of nucleic acid molecules of the nucleic acid sample. In some embodiments, the one or more probes comprise a fluorescent modification. In some embodiments, the one or more probes comprise a quencher modification. In some embodiments, the nucleic acid sample comprises at least 1×10⁶ nucleic acid molecules. In some embodiments, the nucleic acid sample comprises at least 1×10⁸ nucleic acid molecules. In some embodiments, the nucleic acid sample comprises at least 1×10¹⁰ nucleic acid molecules. In some embodiments, the nucleic acid sample comprises at least 1×10¹² nucleic acid molecules.

In some embodiments, the method further comprises prior to (a) generating the plurality of partitions by driving a solution comprising the nucleic acid sample through a membrane. In some embodiments, the plurality of partitions are immobilized within a container during (b) and (c). In some embodiments, the container comprises a tube. In some embodiments, (c) comprises scanning cross-sections of the plurality of partitions immobilized within the container. In some embodiments, (b) comprises heating the plurality of partitions. In some embodiments, (c) comprises performing imaging of the subset of the plurality of partitions. In some embodiments, the imaging is performed using an imaging system. In some embodiments, the imaging system comprises light sheet imaging.

In some embodiments, the imaging comprises collecting image data across a set of channels. In some embodiments, the set of channels comprise fluorescence channels. In some embodiments, the set of channels comprise at least 3 channels. In some embodiments, the set of channels comprise at least 4 channels. In some embodiments, the signals comprise fluorescence intensities associated with the plurality of nucleic acid molecules of the nucleic acid sample. In some embodiments, (d) comprises comparing the signals to a lookup table to identify the plurality of nucleic acid molecules of the nucleic acid sample. In some embodiments, the method further comprises prior to (b), incubating the plurality of partitions at a temperature less than or equal to 20° C. for at least 1 hour. In some embodiments, the method further comprises prior to (c), incubating the plurality of partitions at a temperature less than or equal to 20° C. for at least 1 hour. In some embodiments, the method further comprises prior to (d), incubating the plurality of partitions at a temperature less than or equal to 20° C. for at least 1 hour. In some embodiments, the plurality of nucleic acid molecules identified in (d) comprise at least 10,000 nucleic acid molecules. In some embodiments, the plurality of nucleic acid molecules identified in (d) comprise at least 100,000 nucleic acid molecules. In some embodiments, the plurality of nucleic acid molecules identified in (d) comprise at least 1,000,000 nucleic acid molecules. In some embodiments, the plurality of nucleic acid molecules identified in (d) comprise one or more mutations relative to a reference sequence. In some embodiments, the reference sequence comprises a human reference genome. In some embodiments, the reference sequence comprises a healthy human reference genome.

In some embodiments, (b) comprises performing an amplification reaction. In some embodiments, the amplification reaction comprises polymerase chain reaction. In some embodiments, (b) and (c) are completed in no more than 3 hours. In some embodiments, the plurality of nucleic acid molecules identified in (d) comprise at least 10 different loci. In some embodiments, the plurality of nucleic acid molecules identified in (d) comprise at least 20 different loci. In some embodiments, the plurality of nucleic acid molecules identified in (d) comprise at least 30 different loci.

Some platforms, methods, and compositions for performing multiplexed analyses may involve significant infrastructure investment (e.g., in relation to microfluidic platform and detection platform aspects); however, such technologies are limited in relation to: number of targets that can be detected simultaneously; mechanism by which different targets are differentially detected (e.g., as in mechanisms involving primarily signal amplitude-based detection); ability to provide multiplexing capability with a high degree of accuracy for partitioning technologies where the partitions are arranged three-dimensionally (3D) in bulk format (e.g., in a packed configuration in three dimensions); ability to provide multiplexing for partitioning technologies involving an extremely high number of partitions (e.g., greater than 1 million partitions) for digital analyses; and other factors in the context of multidimensional digital analyses.

Accordingly, this disclosure describes embodiments, variations, and examples of systems, methods, and compositions for digital detection of a large number of targets in a high-performance, efficient, and accurate manner, and with less complex instrumentation.

An aspect of the disclosure provides an approach that balances the amplification kinetics between amplicons and distinguishing such amplicons, thereby enabling the broad adoption of high order multiplex PCR panels. The disclosure provides a new paradigm in PCR amplification and multiplexed detection using UltraPCR, where an example of the embodiment(s) utilizes a centrifugation workflow to split a PCR reaction into ~34 million partitions (or greater), forming an optically clear pellet of spatially separated reaction compartments in a container (e.g., PCR tube). After in-situ thermocycling, light-sheet scanning is used to produce a 3D reconstruction of the fluorescent positive compartments within the pellet. At some sample DNA concentrations, the magnitude of partitions offered by UltraPCR dictate that the vast majority (or all) of the target molecules occupy a compartment uniquely, in single-molecule format. This single molecule realm allows for isolated amplification events, thereby eliminating competition between different targets and generating unambiguous optical signals for detection. Using a 4-color optical setup, the example of the embodiment(s) incorporates 10+ different fluorescent dyes in the same UltraPCR reaction, and push multiplexing to an unprecedented level by combinatorial labeling with fluorescent dyes. Using the same 4-color optical setup, the example of the embodiment(s) incorporate a 22-target comboplex panel that can detect all targets simultaneously at high precision. Collectively, UltraPCR pushes PCR applications beyond is currently available, enabling a new class of precision assays.

The disclosure thus covers systems and/or methods that achieve comparable or better performance of, digital PCR (dPCR) technologies, quantitative PCR (qPCR) technologies, and next generation sequencing (NGS) technologies, within a single platform.

As such, an aspect of the disclosure provides compositions, methods, and systems for implementation of highly multiplexed molecular diagnostic assays involving color combinatorics, stimulus-responsive probes, tandem probes, conjugated polymer probes, and other mechanisms for increasing the number of targets that can be simultaneously detected in a digital assay. As described in more detail below, combinations of mechanisms can provide a number of targets that can be differentially detected according to n!/[r! (n−r)!], where n represents the number of available colors, and r represents the number of selected colors from the number of available colors. Permutations of mechanisms can provide a number of targets that can be differentially detected according to n!/[(n−r)!]+n, where n represents the number of available colors, and r represents the number of selected colors from the number of available colors. In examples, the numbers of targets that can be differentially tagged and detected from a single sample and within a single assay run can be greater than 10 targets, greater than 15 targets, greater than 20 targets, greater than 25 targets, greater than 30 targets, greater than 35 targets, greater than 40 targets, greater than 45 targets greater than 50 targets, greater than 55 targets, greater than 60 targets, greater than 65 targets, greater than 70 targets, greater than 75 targets, greater than 80 targets, greater than 85 targets, greater than 90 targets, or greater than 100 targets, with optical detection of signals from targets.

Differential detection is achieved in part due to the high number of partitions involved when using the technologies described, where distribution of sample targets across partitions results in low occupancy of partitions by targets, and large partition numbers contribute to significantly low percentages of doublets (e.g., single partitions occupied by two targets), triplets (e.g., single partitions occupied by three targets), or other forms of multi-plets (single partitions occupied by multiple targets). In particular, successful multiplexing at this level is attributed to the high degree of partitioning (with achievable numbers of generated partitions described) and extremely low occupancy (with achievable percent occupancies described), such that multiple molecules from the target molecules of interest have a minimal (or zero) probability of occupying the same partition as another target molecule. In such a high-partition and low-occupancy regime, there is no competition associated with multiple target molecules per partition, and the platform is not subject to problems related to differences in PCR efficiency between different target molecules.

In the context of digital multiplexed analyses, the disclosure also provides systems, methods, and compositions that can achieve a high dynamic range, due to the number of partitions involved and occupancy of the partitions by targets of the sample. In examples, the systems, methods, and compositions can provide a dynamic range of: over 4 orders of magnitude from a lower count capability to a higher count capability (e.g., at least 10⁴), over 5 orders of magnitude from a lower count capability to a higher count capability (e.g., at least 10⁵), over 6 orders of magnitude from a lower count capability to a higher count capability (e.g., at least 10⁶), over 7 orders of magnitude from a lower count capability to a higher count capability (e.g., at least 10⁷), or greater, for sample volumes described. In examples, the systems, methods, and compositions can achieve quantification of targets over a 4-log dynamic range, over a 5-log dynamic range, over a 6-log dynamic range, over a 7-log dynamic range, or greater, for sample volumes described.

For partitions arranged in bulk (e.g., in close-packed format, in the form of droplets of an emulsion) within a closed container, the systems, methods, and compositions described can provide discernable signals from individual partitions, with readout performed using multiple color channels (e.g., 2 color channels, 3 color channels, 4 color channels, 5 color channels, 6 color channels, 7 color channels, etc.) corresponding to light sources and optics involved in detection, with suitable signal-to-noise (SNR) characteristics in relation to background fluorescence.

For multiplexed analyses, methods described involve detection of signals from a large number of partitions, where detected signals correspond to a set of color combinatorics paired with targets of a set of targets potentially represented in the sample and contained within partitions of the set of partitions, and wherein the set of targets has a total number greater than the number of color channels used to detect colors corresponding to the set of color combinatorics. In examples, the set of color combinatorics involves combinations of up to 3 colors, up to 4 colors, up to 5 colors, up to 6 colors, up to 7 colors, or greater (from each of the set of partitions), where each combination of colors has a corresponding target associated with the respective combination.

In one embodiment, the set of partitions involves droplets of an emulsion within a closed container, and the set of color combinatorics involves combinations of up to 3 colors, up to 4 colors, up to 5 colors, up to 6 colors, up to 7 colors, etc. detectable from each of the set of partitions. Additionally or alternatively, multiplexing involving stimulus-responsive materials can expand the number of targets that can be differentially tagged and detected by a factor equal to the number of states through which probes used to tag targets can transition. Additionally or alternatively, multiplexing involving materials that exhibit Foerster resonance energy transfer (FRET) behavior can expand the number of targets that can be differentially tagged and detected by a factor equal to the number of FRET capable probes used.

The disclosure also provides compositions that produce significantly improved signal-to-noise (SNR) values with reduced background, in relation to detection techniques described below (e.g., based on light sheet imaging, etc.) for partitions arranged in bulk in 3D. In examples, target signals can be at least 10² greater than background noise signals, 10³ greater than background noise signals, 10⁴ greater than background noise signals, 10⁵ greater than background noise signals, 10⁶ greater than background noise signals, 10⁷ greater than background noise signals, or better. Background noise can be attributed to fluorescence from adjacent partitions and adjacent planes of the set of planes of partitions in the context of emulsion digital PCR, or attributed to other sources with closely-positioned partitions.

In examples associated with reaction materials described and used for droplet digital PCR, determining the target signal value can include: for each plane of a set of planes of partitions under interrogation (e.g., by light sheet detection, by another method of detection, etc.): determining a categorization based upon a profile of positive partitions represented in a respective plane, determining a target signal distribution and a noise signal distribution specific to the profile, and determining a target signal intensity and a noise signal intensity for the respective plane. Here, the target signal value can be an average value (or other representative value) of the target signal intensities determined from the set of planes, and the background noise signal value can be an average value (or other representative value) of the noise signal intensities determined from the set of planes.

The disclosure also provides oligonucleotide compositions and designs for multiplexed assays (e.g., locked nucleic acid (LNA) assays, KASP assays, Taqman assays, etc.). Such improved oligonucleotides improve sample processing, with respect to primer cleanup/removal, reduction of background, implementation of compatible forward and reverse primers for direct multiplexed assays (e.g., PCR), implementation of checks for complementarity of amplicons to non-self probes (i.e., in both sense and antisense strands), implementation of checks for complementarity of primers to probes (i.e., in both sense and antisense strands), generation of positive and negative controls for a clinical workflow, establishment of limits of detection (LoDs) and other metrics for NIPT ultraPCR assays, and other improvements.

Examples of partition generation methods can include generating an extremely high number of droplets (e.g., greater than 5 million droplets, greater than 6 million droplets, greater than 7 million droplets, greater than 8 million droplets, greater than 9 million droplets, greater than 10 million droplets, greater than 15 million droplets, greater than 20 million droplets, greater than 25 million droplets, greater than 30 million droplets, greater than 40 million droplets, greater than 50 million droplets, greater than 100 million droplets, etc.) within a collecting container having a volumetric capacity (e.g., less than 50 microliters, from 50 through 100 microliters and greater, etc.), where droplets have a characteristic dimension (e.g., from 1-50 micrometers, from 10-50 micrometers, etc.) that is relevant for digital analyses, target detection, individual molecule partitioning, or other applications. In embodiments, droplets that form after leaving the side of the membrane facing the liquid layer(s) in the collecting container then pass through the liquid layer(s), in order to form a gel material comprising partitions (e.g., a partition network, a permeable partition network) where the partitions are stabilized in position in the collecting container. The gel material can thus be re-scanned for each of the set of channels of interrogation, while the partitions retain their relative positions within the closed collecting container during each scanning run.

In relation to occupancy, embodiments, variations, and examples of partitioning may be conducted in a manner such that each partition has one or zero molecules (e.g., one or zero target molecules), such that the partitions may be characterized as having low occupancy (e.g., less than 15% occupancy of partitions by individual molecules, less than 14% occupancy of partitions by individual molecules, less than 13% occupancy of partitions by individual molecules, less than 12% occupancy of partitions by individual molecules, less than 11% occupancy of partitions by individual molecules, less than 10% occupancy of partitions by individual molecules, less than 9% occupancy of partitions by individual molecules, less than 8% occupancy of partitions by individual molecules, less than 7% occupancy of partitions by individual molecules, less than 6% occupancy of partitions by individual molecules, less than 5% occupancy of partitions by individual molecules, less than 4% occupancy of partitions by individual molecules, etc.).

In examples, the systems, methods, and compositions described can be used to generate 50,000 counts per target for each of a set of targets of interest, 60,000 counts per target for each of a set of targets of interest, 70,000 counts per target for each of a set of targets of interest, 80,000 counts per target for each of a set of targets of interest, 90,000 counts per target for each of a set of targets of interest, 100,000 counts per target for each of a set of targets of interest, 120,000 counts per target for each of a set of targets of interest, 130,000 counts per target for each of a set of targets of interest, 140,000 counts per target for each of a set of targets of interest, 150,000 counts per target for each of a set of targets of interest, 160,000 counts per target for each of a set of targets of interest, 170,000 counts per target for each of a set of targets of interest, 180,000 counts per target for each of a set of targets of interest, 190,000 counts per target for each of a set of targets of interest, 200,000 counts per target for each of a set of targets of interest, 210,000 counts per target for each of a set of targets of interest, 220,000 counts per target for each of a set of targets of interest, 230,000 counts per target for each of a set of targets of interest, 240,000 counts per target for each of a set of targets of interest, 250,000 counts per target for each of a set of targets of interest, 260,000 counts per target for each of a set of targets of interest, 270,000 counts per target for each of a set of targets of interest, 280,000 counts per target for each of a set of targets of interest, 290,000 counts per target for each of a set of targets of interest, 300,000 counts per target for each of a set of targets of interest, or other counts per target for each of a set of targets of interest.

Compositions, methods, and systems described can further involve use of a single primer with tandem adapters or multiple primers used to tag targets with probes. Multiplexed primers structured to flank target-specific probes that encode for different targets can be used. Multiplexed primer compositions can be configured for 20-plex amplification of loci of interest for each a set of targets being analyzed, 30-plex amplification of loci of interest for each a set of targets being analyzed, 40-plex amplification of loci of interest for each a set of targets being analyzed, 50-plex amplification of loci of interest for each a set of targets being analyzed, 60-plex amplification of loci of interest for each a set of targets being analyzed, 70-plex amplification of loci of interest for each a set of targets being analyzed, 80-plex amplification of loci of interest for each a set of targets being analyzed, 90-plex amplification of loci of interest for each a set of targets being analyzed, 100-plex amplification of loci of interest for each a set of targets being analyzed, or greater.

Relatedly, an aspect of the disclosure provides embodiments, variations, and examples of devices and methods for rapidly generating partitions (e.g., droplets from a sample fluid, droplets of an emulsion) and distributing nucleic acid material (e.g., multiplexed target detection) across partitions, where, the device includes: a first substrate defining a reservoir comprising a reservoir inlet and a reservoir outlet; a membrane coupled to the reservoir outlet and comprising a distribution of holes; and a supporting body comprising an opening configured to retain a collecting container in alignment with the reservoir outlet. During operation, the first substrate can be coupled with the supporting body and enclose the collecting container, with the reservoir outlet aligned with, seated within the collecting container, or a combination thereof. During operation, the reservoir can contain a sample fluid (e.g., a mixture of nucleic acids of the sample and materials for an amplification reaction), where application of a force to the device or sample fluid generates a plurality of droplets at an extremely high rate (e.g., of at least 200,000 droplets/minute, of at least 300,000 droplets/minute, of at least 400, droplets/minute, of at least 500,000 droplets/minute, of at least 600,000 droplets/minute, of at least 700,000 droplets/minute, of at least 800,000 droplets/minute, of at least 900,000 droplets/minute, of at least 1 million droplets/minute, of at least 2 million droplets/minute, of at least 3 million droplets/minute, of at least 4 million droplets/minute, of at least 5 million droplets/minute, of at least 6 million droplets per minute, etc.), where the droplets then pass through the fluid layer(s) and form partitions (e.g., a partition network) may be stabilized in position (e.g., in a close-packed format, in equilibrium stationary positions) within the collecting container.

An aspect of the disclosure provides embodiments, variations, and examples of a method for rapidly generating partitions (e.g., droplets from a sample fluid, droplets of an emulsion) within a collecting container at an extremely high rate, each of the plurality of droplets including an aqueous mixture for a digital analysis, wherein upon generation, the plurality of droplets pass through the fluid layer(s) and form partitions (e.g., a partition network) that are stabilized in position (e.g., in a close-packed format, at equilibrium stationary positions, etc.) within a continuous phase (e.g., as an emulsion having a bulk morphology defined by the collecting container). In aspects, partition generation can be executed by driving the sample fluid through a distribution of holes of a membrane, where the applied force can be one or more of centrifugal (e.g., under centrifugal force), associated with applied pressure, magnetic, or otherwise physically applied.

In relation to a single-tube workflow in which the collecting container remains closed (e.g., the collecting container has no outlet, there is no flow out of the collecting container, to avoid sample contamination), method(s) can further include transmitting heat to and from the plurality of droplets within the closed collecting container according to an assay protocol. In relation to generation of stabilized partitions having suitable clarity (e.g., with or without refractive index matching), method(s) can further include transmission of signals from individual stabilized partitions from within the closed collecting container, for readout (e.g., by an optical detection platform, by another suitable detection platform).

Where method(s) include transmitting heat to and from the plurality of droplets, within the closed container, the droplets may be stable across a wide range of temperatures (e.g., 1° C. through 95° C., greater than 95° C., less than 1° C.) relevant to various digital analyses and other bioassays, where the droplets remain consistent in morphology and remain unmerged with adjacent droplets.

The disclosure generally provides mechanisms for efficient capture, distribution, and labeling of target material (e.g., DNA, RNA, miRNA, proteins, small molecules, single analytes, multianalytes, etc.) in order to enable genomic, proteomic, other multi-omic characterization of materials, or a combination thereof, in parallel and in a multiplexed manner, for various applications.

In examples, the approach discussed is designed around a simple workflow to enable deployment to local and decentralized laboratories. First, samples may be carried end-to-end in the same PCR tube for user convenience and to minimize sample contamination. Second, ultra-partitioning and PCR amplification can be performed using laboratory equipment such as a swing bucket centrifuge and thermal cycler, lowering the infrastructure cost for ultraPCR adoption. However, compositions of the disclosure can also be utilized in coordination with various technologies for isolating material in single-molecule format (e.g., by use of wells, by use of droplets, by use of other partitioning elements, etc.).

Another aspect of the present disclosure provides a non-transitory computer readable medium comprising machine executable code that, upon execution by one or more computer processors, implements any of the methods above or elsewhere herein.

The disclosure provides compositions, methods, and systems for multiplexed detection of targets that can provide value in research or other non-clinical settings, with or without evaluation and processing of live human or mammalian biological material, and without the immediate purpose of obtaining a diagnostic result of a disease or health condition.

Another aspect of the present disclosure provides a system comprising one or more computer processors and computer memory coupled thereto. The computer memory comprises machine executable code that, upon execution by the one or more computer processors, implements any of the methods above or elsewhere herein.

In an aspect, the present disclosure provides a method comprising: performing a digital multiplexed analysis of a sample distributed across a set of more than 500,000 partitions stabilized in position along three axes within a closed container, wherein performing the digital multiplexed analysis comprises: reacting the sample with a set of processing materials within the set of partitions, wherein each of the set of partitions has less than two targets, and detecting signals from the set of partitions upon performing 3D scanning of the set of partitions with a set of optical channels, wherein each of the set of optical channels comprises a respective emission and excitation configuration, wherein said signals correspond to a set of label combinatorics paired with targets of a set of targets potentially represented in the sample and contained within partitions of the set of partitions, and wherein the set of targets has a total number greater than a number of the set of optical channels used to detect signatures corresponding to the set of label combinatorics, and wherein the set of optical channels comprises at least four channels.

In some embodiments, the set of partitions comprises droplets of an emulsion within a closed container. In some embodiments, the set of label combinatorics involves combinations of a set of labels detectable from each of the set of partitions. In some embodiments, the set of labels comprises a label associated with a class I dye. In some embodiments, the set of labels comprises a label associated with a class II dye. In some embodiments, the set of labels comprises a label associated with a class III dye. In some embodiments, the set of labels comprises a photobleachable dye. In some embodiments, the set of partitions is stabilized in position within a container, the method further comprising moving the container during performance of 3D scanning of the set of partitions.

In some embodiments, 3D scanning of the set of partitions comprises scanning the container sequentially, for a number of scanning runs corresponding to the set of optical channels. In some embodiments, the set of optical channels comprises greater than 10 optical channels. In some embodiments, the set of processing materials comprises, for a target of the set of targets: a primer set comprising: a common primer and a set of target-specific primers comprising a target-specific primer structured to interact with a target region of the target, and a first fluorophore-labeled oligonucleotide corresponding to the flanking sequence, the first fluorophore-labeled oligonucleotide comprising a first fluorophore configured to transmit a first target signal if the target region is amplified. In some embodiments, the set of processing materials comprise, for a first target and a second target of the set of targets: a primer set comprising: at least one primer structured to tag the first target with a first probe having a first fluorophore and the second target with a second probe having a second fluorophore. In some embodiments, the first fluorophore is a photo-bleachable fluorophore, and wherein detecting signals from the set of partitions comprises scanning the set of partitions with a first wavelength range of light and a second wavelength range of light configured to bleach the first fluorophore, the method further comprising: detecting signals from the set of partitions in a first phase of analysis upon scanning the set of partitions with the first wavelength range of light, and detecting signals from the set of partitions in a second phase of analysis upon scanning the set of partitions and bleaching the first fluorophore with the second wavelength range of light, thereby enabling differential detection of the first target and the second target.

In some embodiments, detecting a target of the set of targets comprises assembling an optical signature from a partition containing the target, from positive and negative signals aggregated from scanning the partition with each of the set of optical channels. In some embodiments, no partitions of the set of partitions contains two or more targets, and wherein the method omits deconvolution of partitions ambiguously containing multiple targets.

In another aspect, the present disclosure provides a method comprising: performing a digital multiplexed analysis of a sample distributed across a set of partitions stabilized in position within a closed container, wherein each partition of the set of partitions comprises less than two targets of the sample, and wherein the digital multiplexed analysis can simultaneously and differentially detect at least 20 different targets from the sample.

In some embodiments, performing the digital multiplexed analysis comprises: reacting the sample with a set of processing materials within the set of partitions, wherein each of the set of partitions has less than two targets, and detecting signals from the set of partitions upon scanning the set of partitions sequentially with a set of optical channels, wherein said signals correspond to a set of label combinatorics paired with targets of a set of targets potentially represented in the sample and contained within partitions of the set of partitions. In some embodiments, the set of partitions comprises greater than 30 million droplets stabilized in position in an emulsion, and wherein scanning comprises performing a light sheet imaging operation. In some embodiments, the set of targets has a total number greater than a number of the set of optical channels used to detect signatures corresponding to the set of label combinatorics, and wherein the set of optical channels comprises at least 10 channels. In some embodiments, each of the set of optical channels comprises a respective emission and excitation configuration. In some embodiments, the set of partitions comprises greater than 500,000 partitions and wherein the set of partitions is characterized by less than 15% occupancy of partitions by said biological targets.

In some embodiments, wherein the set of processing materials comprises a set of non-hydrolysis probes, the method further comprising tagging the set of targets with a set of permutations of the set of non-hydrolysis probes, wherein detecting signals from the set of partitions comprises detecting signals corresponding to the set of permutations for differential detection of the set of targets. In some embodiments, the method further comprises returning an output supporting at least one of: pathogen detection, non-invasive prenatal testing, organ transplantation analysis, forensics, microbiome analysis, and oncology, based upon the digital multiplexed analysis.

Methods and compositions disclosed herein may be useful for analyzing large amounts of nucleic acids.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A depicts a flowchart of an embodiment of a method for multiplexed detection and digital quantitation of targets.

FIG. 1B depicts a flowchart of a variation of a method for multiplexed detection and digital quantitation of targets, with applications in characterizing non-self genetic material from a sample.

FIG. 1C depicts a flowchart of a variation of a method for multiplexed detection and digital quantitation of targets, with applications in characterizing fetal fraction in a sample.

FIG. 1D depicts a flowchart of portions of an embodiment of a method for multiplexed detection and digital quantitation of targets.

FIG. 2 depicts a schematic of components implemented in an embodiment of a method for multiplexed detection of targets.

FIG. 3A depicts a schematic of differences in multiplexing with low-partition systems in comparison to high-partition systems.

FIG. 3B depicts a schematic of different strategies for labeling a target with one or more probes.

FIG. 3C depicts a schematic of example color combinations for differential detection and quantitation of targets.

FIG. 3D depicts a schematic of example color combinations for differential detection and quantitation of targets.

FIG. 3E depicts a schematic of an emulsion where targets within partitions are tagged in a manner involving color combinatorics.

FIG. 3F depicts variations of tagging a target with tandem probes, in relation to positioning of fluorophores and quenchers.

FIG. 3G depicts scenarios involving use of tandem probes and amplitude-based differentiation of signals involving two colors.

FIG. 3H depicts variations of tagging different targets of a sample with single probes or tandem probes.

FIG. 3I depicts a variation of multiplexing involving probes capable of FRET behavior.

FIG. 3J depicts a schematic of an example of a FRET-capable probe.

FIG. 3K depicts a schematic of an example of stimulus-responsive probes used for tagging and detection of different targets.

FIG. 4A depicts a schematic of achievable levels of multiplexing, with combinations and permutations of probes and detected colors.

FIG. 4B depicts a schematic demonstrating expansion of multiplexing ability for an assay, with combinations of multiplexing strategies.

FIG. 5A depicts alternative assay chemistry for performing differential detection and quantitation of targets in a multiplexed manner.

FIG. 5B depicts an embodiment of a process for determining a signal to noise ratio (SNR) for an embodiment of a digital multiplexed analysis.

FIG. 6 depicts a schematic of an embodiment of a system for partitioning samples.

FIG. 7 depicts schematics of portions of an embodiment of a method for detection of one of the targets in a multiplex panel.

FIG. 8 depicts a schematic related to digital quantitation of targets (e.g., where the number of targets is greater than the number of fluorescent channels).

FIG. 9 illustrates a computer system that is programmed or otherwise configured to implement methods provided herein.

FIG. 10 Overview of UltraPCR. (A) Workflow diagram outlining how UltraPCR works in a 4-step process. Step 1: PCR mix is loaded onto the spin column. Step 2: Centrifugation draws the PCR mix through a custom-designed column to divide a 50 μL reaction into ~34 million partitions within 20 minutes. Step 3: Thermal cycling of the UltraPCR emulsion amplifies signal in partitions with single molecule templates. Step 4: UltraPCR Imager analyzes the optically clear UltraPCR emulsion by translating a laser light sheet through the PCR tube. (B) Plot displaying the % of partitions encompassing more than 1 molecule in legacy dPCR systems (20,000 partitions, black) (15, 20, 21) vs UltraPCR (34,000,000 partitions, blue) (14). In UltraPCR, even at 1,000,000 molecule input, ~99.95% of partitions are either empty or with single target molecules, which enables a paradigm shift of massively parallel single molecule PCR. Schematic of UltraPCR DNA occupancy mimics the actual proportion of partitions with 1 million molecules in 34 million partitions (2.9% occupancy). On the other hand, in legacy dPCR systems, even with 50,000 molecule input, more than 70% of the partitions are occupied with two or more target molecules (250% occupancy), as shown in the schematic.

FIG. 11 Generation of optical signature with spatial information using 10 dyes. (A) Schematic of how optical signature is captured. In this specific example, every partition is scanned serially with 10 different channel settings configured for dye classes I, II, and III. Channel settings include distinct excitation wavelength, indicated by colored vertical lines, emission wavelength, indicated by colored rectangles, and photobleaching events. Data generated in each channel is used to assemble an optical signature for each partition composed of fluorescence intensity values, which unambiguously identifies the target captured in 3D partition positions. Sample of data is shown in the table (B) Light sheet images with photobleachable (FAM, Bodipy TMR-X and Cy5) and photostable (Alexa488, HEX and Atto647N) dyes before and after a brief photobleaching event. Zoomed in micrographs of individual partitions are shown in the corner of images as examples. (C) UMAP visualization of optical signatures for all positive partitions detected by the UltraPCR Imager analysis pipeline followed by clustering using DBScan (22). (D) Aggregate optical signature for each cluster (vertical-axis) with circle size indicating normalized intensity value for each channel (horizontal-axis). (E) Number of molecules identified per cluster. This experiment was performed as a duplicate to generate an error bar signifying standard error.

FIG. 12 Comboplex overview. Strategy for labeling a target with more than one dye. (A) In this design, a target was amplified using 1 to 3 hydrolysis probes using the same sequence but conjugated with different color dyes (shown in the bar above the image, labeled A, B, and C), amplifying a gene segment of prfA of L. cytomonogenes, resulting in 1 color, 2 color, or 3 color combinations. Sample light sheet images in each channel shown in different rows display positive partitions in each color combination. (B) Each color combination generates a unique optical signature that can be visualized on UMAP. (C) Plot of each channel's fluorescence intensity, indicated by size of the circles, for each comboplex configuration; cluster numbers in the horizontal axis correspond to cluster numbers on UMAP.

FIG. 13 Ultra-high multiplex panel using comboplex in UltraPCR. (A) Design of a 22-plex pathogen identification panel, showing the assignment of the 8 fluorescent dyes (labeled A, B, C, D, E, F, G, H, each associated with a unique dye). (B) Simultaneous visualization of the reference optical signatures of each of the 22 targets using UMAP. This reference map was generated by combining optical signatures of all positive partitions from 22 UltraPCR reactions, each performed with one of the 22 synthetic template targets. (C) Different numbers of targets (left) 4, (middle) 8, (right) 22 were spiked into the reaction to show that the reference signatures can be used for pathogen identification without the need for manual analysis and target assignment as in some dPCR methods. Each partition in the sample with all 22 targets was mapped to the nearest clusters of the reference signature to derive the molecule count detected per target in (D). Bar graph displaying the average molecule counts for each of the 22 targets all present in the reaction across 4 technical replicates, and the error bar displaying the standard error.

FIG. 14 shows A) signal intensity across different scanning channels for different dyes, and B) counts of molecules per 50 μL, for each cluster.

FIG. 15 shows A) median signal intensity vs. # of color labels; B) molecules per 50 μL for each color label configuration.

FIG. 16 shows A) Molecules per 50 μL for each dilution level and B) molecules per 50 μL for each dilution level.

FIG. 17 shows a schematic for performing a qPCR amplification reaction.

FIG. 18A shows a schematic of probe designs.

FIG. 18B shows a schematic of probe designs

FIG. 19 shows a schematic for performing a qPCR amplification reaction.

FIG. 20 shows images of tubes containing two different fluid layers.

FIG. 21 shows an image of loading collection containers into a centrifuge bucket.

FIG. 22 shows a diagram depicting sample loading into a container.

FIG. 23 shows a diagram depicting reagents loaded into a partition generation device.

FIG. 24 shows an image of containers with a sample comprising a nucleic acid sample.

FIG. 25 shows primers.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### General Overview

The method(s) described can confer several benefits over other systems, methods, and compositions.

In particular, the method(s) enable detection and digital quantitation of a set of targets having a number much greater than the number of channels (e.g., color channels, fluorescence detection channels) available for detection. Multiplexed detection involving a greater number of targets than available color channels for detection is based upon one or more of: color combinatorics, stimulus-responsive probes, tandem probes, conjugated polymer probes, and other mechanisms for increasing the number of targets that can be simultaneously detected in a digital assay. Such functionality is attributed to operation in a regime involving low occupancy of a large number of partitions, such that there is an extremely low probability of overlap between target template molecules within individual partitions. Large partition numbers contribute to significantly low percentages of doublets (e.g., single partitions occupied by two targets), triplets (e.g., single partitions occupied by three targets), or other forms of multi-plets (single partitions occupied by multiple targets). As such, signals from different amplified target templates distributed across individual partitions can be differentially detected and analyzed in relation to performance of digital assays.

In the context of emulsion digital PCR with partitions retained in bulk within a container, the method(s) achieve improved signal-to-noise (SNR) with respect to detection of signals from a partition surrounded in three dimensions by other partitions also potentially emitting signals, where the partitions may be interrogated by a three dimensional imaging technique. Some assay chemistries (e.g., such as EvaGreen chemistry, SYBR chemistry) may be less appropriate for such applications as they can yield high levels of background noise that reduce assay performance.

In the context of digital multiplexed analyses, the disclosure also provides systems, methods, and compositions that can achieve a high dynamic range, due to the number of partitions involved and occupancy of the partitions by targets of the sample. In examples, the systems, methods, and compositions can provide a dynamic range of: over 4 orders of magnitude from a lower count capability to a higher count capability (e.g., at least 10⁴), over 5 orders of magnitude from a lower count capability to a higher count capability (e.g., at least 10⁵), over 6 orders of magnitude from a lower count capability to a higher count capability (e.g., at least 10⁶), over 7 orders of magnitude from a lower count capability to a higher count capability (e.g., at least 10⁷), or greater, for sample volumes described. In examples, the systems, methods, and compositions can achieve quantification of targets over a 4-log dynamic range, over a 5-log dynamic range, over a 6-log dynamic range, over a 7-log dynamic range, or greater, for sample volumes described.

For partitions arranged in bulk (e.g., in close-packed format, in the form of an emulsion) within a closed container, the systems, methods, and compositions described can provide discernable signals from individual partitions, with readout performed using multiple color channels (e.g., 2 color channels, 3 color channels, 4 color channels, 5 color channels, 6 color channels, 7 color channels, 8 color channels, etc.), with suitable signal-to-noise (SNR) characteristics in relation to background fluorescence.

For multiplexed analyses, methods described involve detection of signals from a large number of partitions, where detected signals correspond to a set of color combinatorics paired with targets of a set of targets potentially represented in the sample and contained within partitions of the set of partitions, and wherein the set of targets has a total number greater than the number of color channels used to detect colors corresponding to the set of color combinatorics. In examples, the set of color combinatorics involves combinations of up to 3 colors, up to 4 colors, up to 5 colors, up to 6 colors, up to 7 colors, or greater, where each combination of colors has a corresponding target associated with the respective combination.

In one embodiment, the set of partitions involves droplets of an emulsion within a closed container, and the set of color combinatorics involves combinations of up to 3 colors, up to 4 colors, up to 5 colors, up to 6 colors, up to 7 colors, etc. detectable from each of the set of partitions. Additionally or alternatively, multiplexing involving stimulus-responsive materials can expand the number of targets that can be differentially tagged and detected by a factor equal to the number of states through which probes used to tag targets can transition. Additionally or alternatively, multiplexing involving materials that exhibit Foerster resonance energy transfer (FRET) behavior can expand the number of targets that can be differentially tagged and detected by a factor equal to the number of FRET capable probes used.

In the context of emulsion digital PCR with the numbers of partitions described, such multiplexed assay design aspects described can produce significantly improved signal-to-noise (SNR) values with reduced background, in relation to detection techniques described below (e.g., based on light-sheet imaging, etc.). In examples, the ratio of target signals (e.g., median target signals) to noise (e.g., median background noise from other partitions, planes of partitions, or a combination thereof) can be greater than 10², greater than 10³, greater than 10⁴, greater than 10⁵, greater than 10⁶, greater than 10⁷, greater than 10⁸, greater than 10⁹, greater than 10¹⁰, or any intermediate value. Background noise can be attributed to fluorescence from adjacent partitions and adjacent planes of the set of planes of partitions in the context of emulsion digital PCR, or attributed to other sources with closely-positioned partitions. In examples, upon processing a sample with the processing materials, noise (e.g., median noise from other partitions, from other planes of partitions, or a combination thereof) can be reduced in relation to target signals by a factor of at least 10⁻², 10⁻³, 10⁻⁴, 10⁻⁵, 10⁻⁶, 10⁻⁷, 10⁻⁸, 10⁻⁹, 10⁻¹⁰, or any intermediate value.

In variations, processing materials of the method(s) described can include: a primer set comprising a common primer and a target-specific primer (or set of target-specific primers) structured to interact with the target region, the target-specific primer having a common adapter sequence, a fluorophore-labeled oligonucleotide corresponding to the common adapter sequence, the fluorophore-labeled oligonucleotide comprising a fluorophore configured to transmit a target signal if the target region is amplified, and a probe additive reagent structured to reduce background noise (e.g., from adjacent partitions and planes of partitions within the container, as described). The common primer can be a forward primer or a reverse primer. The target-specific primer can be a forward primer or a reverse primer. In specific examples, processing materials can include Taqman® probes (e.g., dual-labeled hydrolysis probes). In specific examples, processing materials can include molecular beacons or similar probe structures (e.g., probes with hairpin structures), where such probes may be quenched by quenchers at regions opposite respective fluorophores when the probe is not bound to a target or in an extended configuration.

The methods provide high degrees of multiplexing for detection and digital counting of different targets in parallel, using color combinatorics in a non-sequencing-based system (e.g., non-next generation sequencing (NGS)-based system), in a dilution regimen. In specific examples, the methods can provide high degrees of multiplexing for characterization of different targets in parallel, using color combinatorics in a partition-based PCR system capable of providing an ultra-high number of partitions, thereby reducing/eliminating signal overlap and contributing to unambiguous assignment of partition contents for detection and digital quantitation.

In more detail, the methods confer the benefit of enabling performance of ultra-high multiplexed target detection using a partitioning system and methods of sample processing configured to provide a high number of partitions (e.g., more than 100,000 partitions, more than 200,000 partitions, more than 500,000 partitions, more than 1 million partitions, more than 10 million partitions, more than 20 million partitions, more than 30 million partitions, more than 50 million partitions, more than 100 million partitions, etc.) with low-occupancy (e.g., less than 10% occupancy, less than 8% occupancy, less than 5% occupancy, etc.) of partitions by targets. In particular, use of a low-occupancy platform involving high numbers of partitions provides a regime where the probability of encountering a falsely-labeled multi-color partition associated with one of a set of targets is very low. Such a regime allows a high number of targets to be uniquely labeled by at least one color at low error.

In various applications, the methods provide functionality for assaying samples for a panel of SNPs, CNVs, insertions, deletions, targets associated with other loci of interest, other suitable components, or a combination thereof. Evaluation of samples is performed in a multiplexed manner/in parallel, instead of detecting targets one-by-one in separate reactions.

In various applications, the method(s) can evaluate samples for a panel of SNPs (e.g., 50 common SNPs, less than 50 common SNPs, greater than 50 common SNPs). In some cases, the methods may not comprise sequencing of an individual first to determine the target panel. Common SNPs may be those that have allele frequency of 1% or more (e.g., from 30-60% allele frequency) in the population.

In one specific use case, the methods can be used to determine an amount of non-self vs. self genetic material in a sample mixture (e.g., from relative abundance calculations), where self genetic material originates from a subject, and non-self genetic material originates from another subject, and both the self genetic material and non-self genetic material may be mixed within the same sample. Determination of non-self vs. self genetic material can have specific uses in one or more of: non-invasive prenatal testing (NIPT) and non-invasive prenatal screening (e.g. for measuring fetal DNA fraction in maternal blood); evaluation or prediction of success of organ transplants (e.g. predicting rejection events by monitoring level of donor DNA in recipient's blood); evaluation of a sample to characterize DNA associated with cancers and DNA not associated with cancers (e.g., tumor DNA vs. non-tumor DNA); evaluation of a mixture of environmental samples (e.g., for detection of genetically modified organisms); forensic applications (e.g., detection of minute amounts of suspect DNA in a sample, which is difficult to detect by implementation of other PCR platforms); and other use cases.

In another specific use case, the method(s) can be used for evaluation of minimal residual disease (MRD) based upon detection of numbers of cancer cell targets present in a sample from a subject after one or more phases of cancer treatment (e.g., treatment of leukemia, treatment of lymphoma, treatment of multiple myeloma, etc.).

In another specific use case, the method(s) can be used for single nucleotide polymorphism genotyping (SNPtyping) to measure genetic variations of SNPs between members (e.g., members of a species). Additionally, the method(s) can be used for single nucleotide variant genotyping (SNVtyping) for germline DNA samples.

The method(s) can also be used for applications involving disease prediction generation and monitoring with multiplexed detection of markers of a gene expression marker panel (e.g., for pregnancy-associated complications, for other applications).

In another specific use case, systems, methods, and compositions described can be useful for ribosomal 16S, ITS characterization, or a combination thereof. In relation to the specific use case, systems, methods, and compositions described can be used to disperse a sample of 16S, ITS ribosomal RNA (rRNA) across a plurality of partitions, or a combination thereof (as described in more detail below), where processing materials described enable detection of regions/sequences of interest (e.g., V3 region, V4 region, V5, region, other hypervariable regions, etc.), and subsequently, for operational taxonomic unit (OTU) or amplicon sequence variant (ASV) categorizations. For instance, detection of V3, V4, V5, or a combination thereof regions can be used for bacterial microbiome analyses, fungal microbiome analyses, other microbiome analyses, rare species detection, other applications, or a combination thereof. Additionally or alternatively, such rRNA characterizations can be used for antimicrobial susceptibility testing (e.g., with a sample having one or more antibiotics being assessed, combined with bacteria and materials that can be used to indicate bacteria responses to the antibiotic(s)). Additionally or alternatively, such rRNA characterizations can be used for detection of a set of pathogens (e.g., up to 30 pathogens, up to 40 pathogens, up to 50 pathogens, up to 60 pathogens, up to 70 pathogens, etc.) and quantification (e.g., in relation to detection of presence or absence of various pathogens, in relation to characterization of infectious agents and potential prognoses). Additionally or alternatively, for microbial pathogen detection/quantification, any part of microbial genomics of a sample (e.g., non-rRNA targets) can be targeted, and subsequent detection can involve detection of sample composition (e.g., microbial composition, microbiome composition, etc.) without performance of next generation sequencing (NGS). In a related use case, detection/quantification of targets of a sample in a multiplexed manner can be used to differentiate between viral, fungal, and/or microbial infections (e.g., for a respiratory illness panel).

Additionally or alternatively, the method(s) can provide functionality for detection of other target analytes in a differentiable and multiplexed manner. In examples, analytes can include one or more of: DNA (e.g., cell-free DNA, genomic DNA, etc.), RNA, miRNA, proteins, small molecules, single analytes, multianalytes, chemicals, other analytes, or a combination thereof. For nucleic acid targets, capture probes of compositions described can include complementary molecules to the nucleic acid targets. For protein targets or small molecule targets, capture probes of the compositions described can include antibodies or aptamers conjugated with specific nucleic acid sequences for detection.

In another specific use case, the method(s) can provide functionality for detecting and quantifying a panel of synthetic targets, for instance, in order to provide quality controls for biological standards used to standardize assay performance with multiple synthetic targets in different concentrations. In another specific use case, nucleic acids exogenously introduced into cells (e.g., such as in genome editing applications, such as in CRISPR applications) can be measured using the method(s) described (e.g., for genome editing applications, for gene therapy applications).

The method(s) can be applied to samples from human organisms, other multicellular animals, plants, fungi, unicellular organisms, viruses, other material, or a combination thereof, with respect to evaluating presence or absence of sets of targets in parallel. Characterizations of the sets of targets can then be used for diagnostic purposes, for generation of targeted therapies to improve states of organisms from which the samples were sourced, or a combination thereof. The method(s) can also provide value in research or other non-clinical settings, with or without evaluation and processing of live human or mammalian biological material, and without the immediate purpose of obtaining a diagnostic result of a disease or health condition.

In combination with a higher number of colors/dyes used for detection, the method(s) can further improve the number of targets that can be detected from a sample within a single container, in a single-tube workflow.

The method(s) confer(s) the benefit of providing non-naturally occurring compositions for facilitating interactions with and amplification of a large set of target analytes from a sample in parallel, with improved efficiency, without utilizing complex microfluidic setups, and in a manner that reduces overall costs. As such, the method(s) provide a cost-competitive alternative to other methods for detection and digital quantitation of a large number of target analytes in a multiplexed manner.

The method(s) can provide mechanisms for target-specific/allele-specific amplification and can be applied to digital polymerase chain reaction (dPCR), other PCR-associated assays, or a combination thereof.

Additionally or alternatively, the method(s) can confer any other suitable benefit.

### 2. Methods and Materials

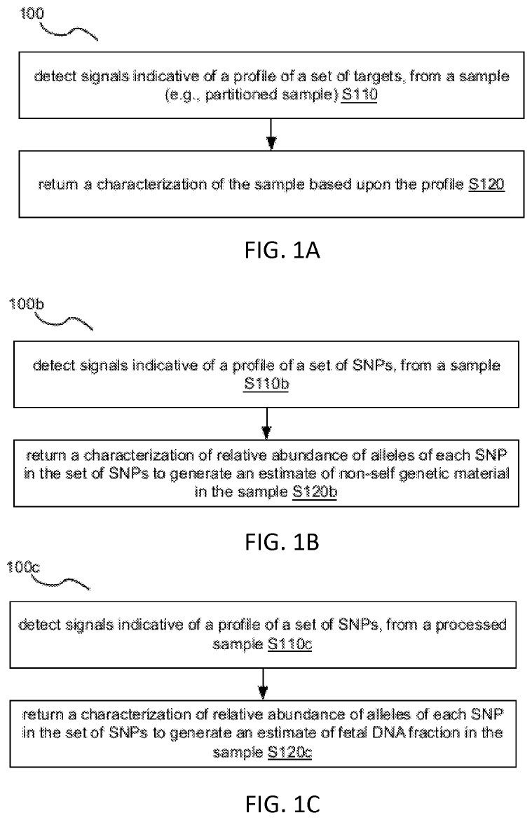

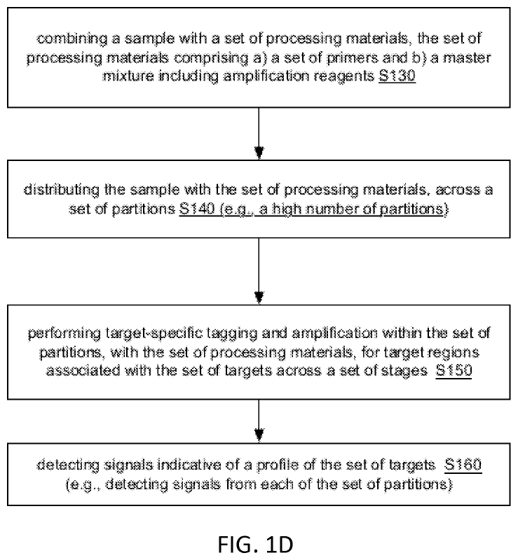

As shown in FIG. 1A, embodiments of a method 100 for multiplexed detection and quantitation of targets includes: detecting signals indicative of a profile of a set of targets, from a sample distributed across a set of partitions (e.g., a high number of partitions at low occupancy) S110, and returning a characterization of the sample based upon the profile S120. In embodiments, said signals correspond to a set of color combinatorics, other differentiable signals resulting from probes used to tag targets, or a combination thereof, wherein color combinatorics of the set of color combinatorics may be paired with targets of the set of targets, and where the set of targets has a total number greater than the number of color channels used to detect colors corresponding to the set of color combinatorics. As such, the method can provide unique labeling for multiplexed characterization of a panel of targets based upon color combinatorics. In some cases, the method may not comprise multiplexing based solely upon signal amplitudes.

In a variation, as shown in FIG. 1B, a method 100b for characterization of non-self genetic material from a sample containing self genetic material and non-self genetic material can include: detecting signals indicative of a profile of a set of set of single nucleotide polymorphisms (SNPs) from a sample distributed across a set of partitions S110b, and returning a characterization of relative abundance of alleles of each SNP in the set of SNPs to generate an estimate of non-self-genetic material in the sample S120b.

In a specific example, as shown in FIG. 1C, a method 100c for determination of fetal fraction (FF) can include detecting signals indicative of a profile of a set of SNPs from a sample distributed across a set of partitions S110c, and returning a characterization of relative abundance of alleles of each SNP in the set of SNPs to generate an estimate of fetal DNA fraction in the sample S120c.

One or more of the methods described can further include steps for processing a sample or providing an environment for producing the processed sample such that it can produce signals indicative of the profile(s), where processing the sample can include (as shown in FIG. 1D): combining a sample with a set of processing materials, the set of processing materials comprising a) a set of primers (e.g., for each of the set of targets, a set of target-specific forward primers corresponding to different targets of the set of targets, and a common reverse primer for the set of target-specific forward primers; other primer designs), and b) a master mixture including amplification reagents S130; distributing the sample with the set of processing materials, across a set of partitions (e.g., a high number of partitions at low occupancy, such that different targets of the set of targets do not co-inhabit a single partition) S140; performing target-specific (e.g., allele-specific) tagging and amplification, with the set of processing materials, for target regions associated with the set of targets across a set of stages S150; and detecting signals indicative of a profile of the set of targets S160.

The methods function to enable detection of genetic variations in biological sample material, in a multiplexed manner. In more detail, the methods enable performance of ultra-high multiplexed target detection by implementing a high number of partitions (e.g., more than 100,000 partitions, more than 200,000 partitions, more than 500,000 partitions, more than 1 million partitions, more than 10 million partitions, more than 20 million partitions, more than 30 million partitions, more than 50 million partitions, more than 100 million partitions, etc.) with low-occupancy (e.g., less than 10% occupancy, less than 8% occupancy, less than 5% occupancy, etc.) of partitions by targets. In particular, use of a low-occupancy platform involving high numbers of partitions provides a regime where the probability of encountering more than one target in a partition is very low, such that a unique color combination can be inferred from the particular target color-coded by the unique color combination.

In relation to detection and effective use of sample processing materials, the method(s) involve detection of signals from targets of interest of a processed sample, where the signals correspond to different color combinatorics of a set of color combinatorics, alone or in combination with other types of differentiable signals, where color combinatorics of the set of color combinatorics may be paired with targets of the set of targets, and where the set of targets has a total number greater than the number of color channels used to detect colors corresponding to the set of color combinatorics. In particular, due to the high-degree of partitioning described, any positive partition (e.g., droplet of an emulsion generated from the sample and containing a target) may contain one color combination corresponding to fluorescent materials used during processing of the sample, thereby providing an accurate mechanism for multiplexed detection.

In specific examples, the method(s) can provide a multi-color combinatoric scheme with 5-color assay. As described in more detail below, the methods can provide mechanisms for multi-color combinatorics using competitive target-specific or allele-specific assays (e.g., Kompetitiv allele-specific PCR (KASP), PCR allele competitive extension (PACE), etc.), other assay chemistries, or a combination thereof because they involve no additional probe sequence within generated amplicons, provide a low degree of assay complexity, and thus result in significantly reduced assay cost for a panel of targets. In variations, such assays can be based upon target-specific (e.g., allele-specific) oligonucleotide extension and fluorescence resonance energy transfer for signal generation. In alternative variations, such assays can be based upon generation and detection of other types of signals.

The methods can further provide functionality for multiplexed detection of genetic variants in a sample by optimizing the amount of information obtained using lower-cost and/or a reduced set of sample processing materials compared to assays based upon fluorescent detection, involving a higher number of primer types, probe types, quencher types, and probe additives. In combination with a higher number of colors/dyes used for detection, the methods can further improve the number of targets that can be detected from a sample within a single container.

The method(s) can be implemented by embodiments, variations, and examples of system components described in U.S. application Ser. No. 17/230,907 filed on 14 Apr. 2021 and/or U.S. application Ser. No. 17/687,080 filed 4 Mar. 2022, which may be each hereby incorporated in its entirety by this reference. Additionally or alternatively, the method(s) can be implemented by other system elements.

Sample Types and Targets: In variations, the method 100 can be used to process sample types including biological fluids including or derived from one or more of: blood (e.g., whole blood, peripheral blood, non-peripheral blood, blood lysate, etc.), plasma, serum, saliva, reproductive fluids, mucus, pleural fluid, pericardial fluid, peritoneal fluid, amniotic fluids, otic fluid, sweat, interstitial fluid, synovial fluid, cerebral-spinal fluid, urine, gastric fluids, biological waste, other biological fluids; tissues (e.g., homogenized tissue samples); food samples; liquid consumable samples; and/or other sample materials. Samples can be derived from human organisms, other multicellular animals, plants, fungi, unicellular organisms, viruses, and/or other material. In specific examples, samples processed can include maternal samples (e.g., blood, plasma, serum, urine, chorionic villus, etc.) including maternal and fetal material (e.g., cellular material, cell-free nucleic acid material, other nucleic acid material, etc.) from which prenatal detection or diagnosis of genetic disorders (e.g., aneuploidies, genetically inherited diseases, other chromosomal issues, etc.) can be performed.

In embodiments, targets detected in a multiplexed manner according to embodiments, variations, and examples of the method 100 can include: nucleic acids (e.g., DNA, RNA, miRNA, etc.), proteins, amino acids, peptides, small molecules, single analytes, multianalytes, chemicals, and/or other target material, in order to enable genomic, proteomic, and/or other multi-omic characterizations and diagnoses for various applications. Genetic targets can include one or more of: single nucleotide polymorphisms (SNPs), copy number variations (CNVs), insertions, deletions, genes, methylated loci, and/or other loci of interest.

In variations, SNPs tagged in a massively parallel manner and detected in a multiplexed manner according to methods described can include SNPs associated with any chromosome having a minor allele fraction (MAF) greater than 0.4. SNPs evaluated can alternatively be characterized by MAF above another suitable threshold (e.g., MAF>0.2, MAF>0.3, etc.). SNPs evaluated can be for coding regions (e.g., synonymous, non-synonymous, missense, nonsense) and/or non-coding regions. SNPs evaluated can be biallelic or multiallelic, with more than two alleles per SNP.

In examples, SNPs can be associated with chromosomes 13, 18, 21, X, Y, and/or other chromosomes, at various loci (e.g., from 10 to 20,000 polymorphic loci); however, SNPs evaluated can additionally or alternatively be associated with other chromosomes and/or loci. Furthermore, the size of the panel of targets can be determined based upon the likelihood of detecting at least one SNP that is homozygous in the mother and heterozygous in the fetus, such that it can be used as a marker for estimation of FF.

The size of the SNP panel being evaluated, threshold MAF for each SNP, and chromosomal distribution can thus be selected to optimize or otherwise increase the probability of returning an accurate estimate of FF or other characterization, based upon the methods described.

Furthermore, SNPs selected for evaluation can have allele pairs that may be well-discriminated (e.g., with respect to stabilizing-destabilizing characteristics). For instance, SNPs can be selected with prioritization of G/T, C/A, and T/A SNPs having high destabilization strength characteristics.

In examples, SNPs can include one or more of: rs2737653 with G/T alleles, rs2737654 with T/G alleles, rs1160680 with C/T alleles, rs701232 with C/T alleles, rs1736442 with A/G alleles, rs7232004 with G/T alleles, rs1498553 with C/T alleles, and other suitable SNPs.

Additionally or alternatively, in other specific applications, target material tagged in a multiplexed manner and evaluated according to methods described can provide diagnostics and/or characterizations in relation to one or more of: monitoring or detection of products (e.g., proteins, chemicals) released from single cells (e.g., interleukins or other compounds released from immune cells); monitoring cell survival and/or division for single cells; monitoring or detection of enzymatic reactions involving single cells; antibiotic resistance screening for bacteria; characterization of pathogens in a sample (e.g., in relation to infections, sepsis, in relation to environmental and food samples, etc.); microbiome characterizations (e.g., based upon detection of hypervariable regions of rRNA); characterization of heterogeneous cell populations in a sample; characterization of individual cells or viral particles; monitoring of viral infections of a single host cell; liquid biopsies and companion diagnostics; detection of cancer forms from various biological samples (e.g., from cell-free nucleic acids, tissue biopsies, biological fluids, feces, etc.) based upon characterization of target panels; detection and/or monitoring of minimal residual diseases; monitoring responses to therapies; detection or prediction of rejection events of transplanted organs; other diagnostics associated with other health conditions; other characterizations of statuses of other organisms; and other suitable applications.

### 2.1 Method—Assay Materials and Compositions

### 2.1.1 Method—Assay Materials and Compositions for Competitive Target-Specific Assays

Step S130 recites combining the sample with a set of processing materials, which functions to tag and amplify multiple targets of the sample in parallel. The set of processing materials described here in Section 2.1.1 can include fewer components (e.g., forward and reverse primers, single primers with tandem adapters, using shared probes/quencher oligonucleotides for primers targeting different targets, etc.) to provide detection of multiple targets in parallel. For instance, for a set of colors/wavelengths used for detection and digital quantitation of multiple targets based on color combinatorics, the set of processing materials may include one probe for each of the set of colors/wavelengths, rather than one probe per target of interest. As such, probes can be designed against a common PCR adapter tagged to forward and/or reverse primers of the set of processing materials, where the number of probes used has a number corresponding to the number of channels for detection, rather than the number of targets, thereby significantly reducing assay cost. As such, the set of processing materials implements chemistry for differential discrimination of partition contents based on color combinatorics, where color combinatorics of a set of color combinatorics may be paired with targets of a set of targets of interest, and where the set of targets has a total number greater than the number of color channels used to detect colors corresponding to the set of color combinatorics.

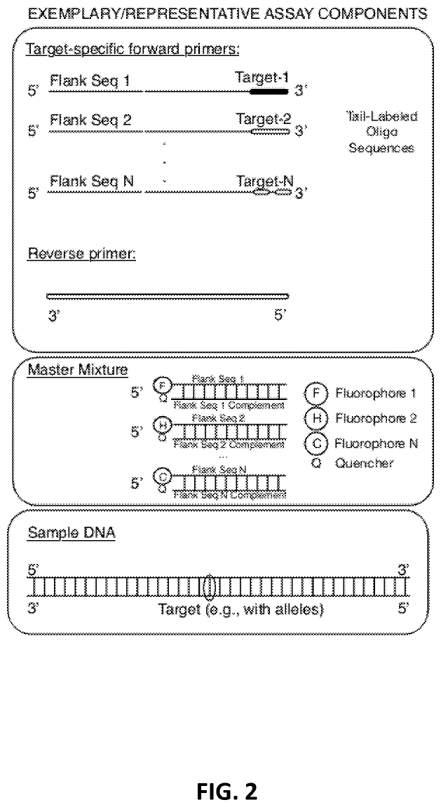

In embodiments, as shown in FIG. 2, the set of processing materials can include: a) for each of the set of targets, a set of target-specific (e.g., allele-specific) forward primers corresponding to different alleles of a respective target of the set of targets, and a common reverse primer for the set of target-specific (e.g., allele-specific) forward primers, and b) a master mixture including amplification reagent as well as: for each of the set of targets, a set of target-specific (e.g., allele-specific) flanking sequences corresponding to different targets of the set of targets.

For each target (e.g., SNP, CNV, loci of interest, insertion, deletion, other target) with allelic variations, the set of target-specific forward primers can include an allele-specific forward primer for each allele. As such, the set of target-specific forward primers can include two allele-specific forward primers, or greater than two allele-specific forward primers (e.g., 3 allele-specific forward primers, 4 allele-specific forward primers, 5 allele-specific forward primers, etc.) for multiallele variations. The allele-specific forward primers include sequence portions complementary to alleles of targets, such that the primer groups encode the target (e.g., SNP, other target) being evaluated, and colors/fluorophores detected provide indication of which allele of a target is present.

The allele-specific forward primers for each target may be configured to be competing, and include unique tail sequences for each allele. In variations, the tail sequences include oligonucleotides with a label corresponding to a dye/fluorophore that can be detected after sample processing. The label can be positioned at the 5′ end of the forward primer or the 3′ end of the forward primer, or can otherwise be positioned (e.g., at a position intermediate the 3′ and 5′ ends). In variations, each forward primer can include multiple labels. The target-specific forward primers can incorporate mismatches at or near penultimate sites (e.g., depending upon destabilization effects of allele combinations associated with targets being evaluated). In variations, the common reverse primer can additionally or alternatively include one or more labels, and/or the set of processing materials can include multiple reverse primers.

Concentrations of forward primers can range from 50 nM to 300 nM in solution, or alternatively, less than 50 nM or greater than 300 nM in solution. Concentrations of reverse primers can range from 100 nM to 600 nM in solution, or alternatively, less than 100 nM or greater than 600 nM in solution. Concentrations of reporter oligonucleotides (e.g., fluorescent reporter oligonucleotides) can range from 30 nM to 200 nM in solution, or alternatively, less than 30 nM or greater than 200 nM in solution. Concentrations of quencher oligonucleotides can range from 100 nM to 600 nM in solution, or alternatively, less than 100 nM or greater than 600 nM in solution.

Primers (e.g., forward primers, reverse primers) can have lengths of 20 base pairs, 21 base pairs, 22 base pairs, 23 base pairs, 24 base pairs, 25 base pairs, 26 base pairs, 27 base pairs, 28 base pairs, 29 base pairs, 30 base pairs, 35 base pairs, 40 base pairs, 45 base pairs, 50 base pairs, an intermediate number of base pairs, or a greater number of base pairs. In variations, primers can incorporate sequence regions corresponding to probes and target sequences (e.g., a 20 base pair target sequence, a target sequence having another suitable length, etc.), and be designed for various levels of plexy (e.g., 1-plex conditions, 2-plex conditions, 3-plex conditions, 4-plex conditions, 5-plex conditions, 6-plex conditions, 7-plex conditions, etc.) as described. In variations, forward primers can be longer than reverse primers, and in specific examples, used of forward primers having lengths 5-10 base pairs longer (e.g., than reverse primers, than another reference length) produced higher counts (e.g., 8-10% higher counts) and higher SNR values (e.g., 12-17% higher SNR values) in relation to shorter primer lengths, when detecting of targets from partitions, thereby providing higher detection performance.

Primers (e.g., forward primers, reverse primers) can have annealing temperatures from 48 C-65 C or another suitable annealing temperature range based upon reactions performed according to various assays. Primers (e.g., forward primers, reverse primers) can have melting temperatures from 65 C to 70 C (e.g., from 67 C to 68.8 C) or another suitable melting temperature range based upon reactions performed according to various assays.

Characteristics of forward and reverse primers described above can be reversed (e.g., the set of processing materials can include a forward primer and a set of target-specific reverse primers). Still alternatively, both forward and reverse primers can be target-specific.

As noted briefly above and shown in FIG. 2, in embodiments, the master mixture can include amplification reagents and, for each of the set of targets, a set of target-specific flanking sequences corresponding to different targets of the set of targets, in order to support multiplexed processing, detection, and digital quantitation. As such, in one variation, the set of processing materials can include, for a target of the set of targets: a primer set comprising: a common primer and a set of target-specific primers structured to interact with a target region of the target, the set of target-specific primers comprising a first target-specific primer comprising a first flanking sequence, and a first fluorophore-labeled oligonucleotide corresponding to the flanking sequence, the first fluorophore-labeled oligonucleotide comprising a first fluorophore configured to transmit a first target signal if the target region is amplified.

For tagging a target with probes configured to emit multiple colors (where tandem probes are described in more detail below), the set of target-specific primers can further include a second target-specific primer comprising a second flanking sequence, and the set of processing materials further comprises a second fluorophore-labeled oligonucleotide corresponding to the second flanking sequence, the second fluorophore-labeled oligonucleotide comprising a second fluorophore configured to transmit a second target signal if the target region is amplified, such that the target can be positively detected based upon the first target signal and the second target signal. Alternatively, a single primer can be used to tag the target, along with tandem adapters corresponding to the probes used to tag the targets. As such, the set of processing materials can include at least one primer structured to tag the target with a first probe having a first fluorophore and a second probe having a second fluorophore (and/or additional probes with additional fluorophores), wherein the first fluorophore and the second fluorophore (and optional additional fluorophores) correspond to two (or more) color channels of the number of color channels.

The master mixture can include a probe including a dye/fluorophore with complementary quencher for each target, a polymerase (e.g., Taq polymerase), dNPTs, and buffer components.

With respect to tagging implemented using the forward primers, and corresponding dyes/fluorophore families of probes, dyes/fluorophores can be associated with chemical families including: acridine derivatives, arylmethine derivatives, fluorescein derivatives, anthracene derivatives, tetrapyrrole derivatives, xanthene derivatives, oxazine derivatives, dipyrromethene derivatives, cyanine derivatives, squaraine derivates, squaraine rotaxane derivatives, naphthalene derivatives, coumarin derivatives, oxadiazole derivatives, pyrene derivatives, and/or other chemicals. Such fluorophores can further be attached to other functional groups for tagging of targets in a detectable manner.

In examples, dyes (e.g., for tagging of RNAs, DNAs, oligonucleotides, etc.) can include one or more of: FAM, (e.g., 6-FAM), Cy3™, Cy5™, Cy5.5™, TAMRA™ (e.g., 5-TAMRA, 6-TAMRA, etc.), MAX, JOE, TET™, ROX, TYE™ (e.g., TYE 563, TYE 665, TYE 705, etc.), Yakima Yellow®, HEX, TEX (e.g., TEX 615), SUN, ATTO™ (e.g., ATTO 488, ATTO 532, ATTO 550, ATTO 565, ATTO Rho101, ATTO 590, ATTO 633, ATTO 647, etc.), Alexa Fluor® (e.g., Alexa Fluor 488, Alexa Fluor 532, Alexa Fluor 546, Alexa Fluor 594, Alexa Fluor 647, Alexa Fluor 660, Alexa Fluor 750, etc.), IRDyes® (e.g., 5′IRDye 700, 5′IRDye 800, 5′IRDye 800CW, etc.), Rhodamine (e.g., Rhodamine Green, Rhodamine Red, Texas Red®, Lightcycler®, Dy 750, Hoechst dyes, DAPI dyes, SYTOX dyes, chromomycin dyes, mithramycin dyes, YOYO dyes, ethidium bromide dyes, acridine orange dyes, TOTO dyes, thiazole dyes, CyTRAK dyes, propidium iodide dyes, LDS dyes, BODIPY dyes, and/or other dyes.

In examples, cell function dyes for tagging of target material and detection can include one or more of: DCFH, DHR, SNARF, indo-1, Fluo-3, Fluo-4, and/or other dyes.

In examples, fluorescent proteins for tagging of target material and detection can include one or more of: cerulean, mCFP, mTurquoise, T-Sapphire, CyPet, ECFP, CFP, EBFP, Azurite, and/or other fluorescent proteins.

Dyes/fluorophores implemented can correspond to wavelength ranges in the visible spectrum and/or non-visible spectrum of electromagnetic radiation. Furthermore, dyes/fluorophores implemented can be configured to prevent overlapping wavelengths (e.g., of emission) and/or signal bleed through with respect to multiplexed detection and achieving high SNR values involving detection of signals from packed partitions. In variations, the set of processing materials can include components for 7 wavelength ranges for multiplexed detection of targets; however, the set of processing materials can include components for less than 7 wavelength ranges (e.g., one wavelength, two wavelengths, three wavelengths, four wavelengths, five wavelengths) or more than 7 wavelength ranges.

Quencher oligonucleotides implemented can include a quencher molecule configured such that, when the quencher oligonucleotide anneals with a primer having a fluorophore, the quencher molecule is in proximity to (e.g., directly opposite) the fluorophore in order to quench the fluorophore. Additionally or alternatively, quenchers can include one or more of: black hole quenchers, static quenchers, self-quenchers (e.g., fluorophores that self-quench under certain conditions by producing secondary structures or other structures), and/or other suitable quenchers. Variations of positions of quenchers (e.g., when tandem probes may be involved) are described in more detail below.

The set of processing materials of Step S130 can additionally or alternatively include implementation of components structured to improve signal-to-noise ratio (SNR) characteristics in the context of multiplexed detection, by increasing signal characteristics and/or reducing background (e.g., noise other artifacts). The components can include one additive for each wavelength range/color for detection (as opposed to one additive for each target/SNP being evaluated). Additionally or alternatively, the additives can have from 5-20 bases or another suitable number of bases. Additionally or alternatively, modified nucleic acids (e.g., such as locked nucleic acids (LNA) or other modified nucleic acids) can be incorporated into forward and/or reverse primers of the set of processing materials to improve SNR. In variations, LNA content can occupy a percentage (e.g., 10-60% LNA content) of the respective primer to improve SNR, where LNA content can be biased toward the 3′ end, the 5′ end, or intermediate the 3′ and 5′ ends.

However, the set of processing materials can additionally or alternatively include other suitable components and/or be configured in another suitable manner.

Furthermore, with respect to different wavelength ranges, different targets can be tagged with dye/fluorophore colors in a manner that promotes discrimination of results (e.g., without overlap) upon detection of signals from processed sample material. Furthermore, different targets can be matched with different combinations of colors/associated wavelengths in order to provide distinction upon detection of signals from processed sample materials. Variations and examples of multiplexing based upon color combinatorics and other features are provided below.

### 2.1.2 Multiplexing Based Upon Color Combinatorics

In relation to Step S130, the method can include detecting signals from the set of partitions, wherein the signals correspond to a set of color combinatorics paired with targets of a set of targets potentially represented in the sample and contained within partitions of the set of partitions, and wherein the set of targets has a total number greater than the number of color channels used to detect colors corresponding to the set of color combinatorics S132. In variations, the set of color combinatorics involves combinations of up to 3 colors detectable from each of the set of partitions, up to 4 colors detectable from each of the set of partitions, up to 5 colors detectable from each of the set of partitions, up to 6 colors detectable from each of the set of partitions, up to 7 colors detectable from each of the set of partitions, up to 8 colors detectable from each of the set of partitions, or another suitable number of colors detectable from each of the set of partitions.

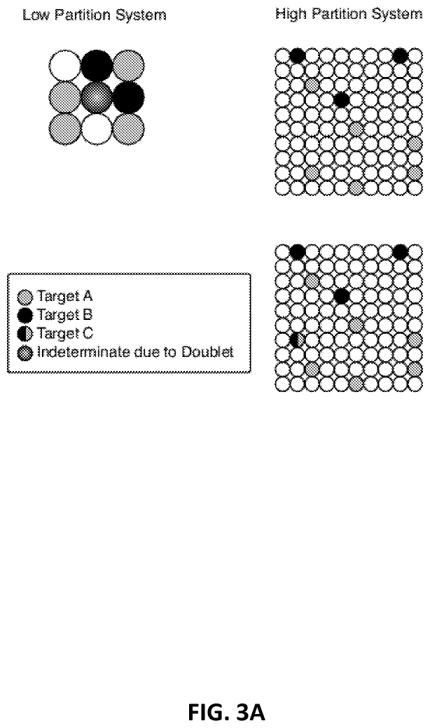

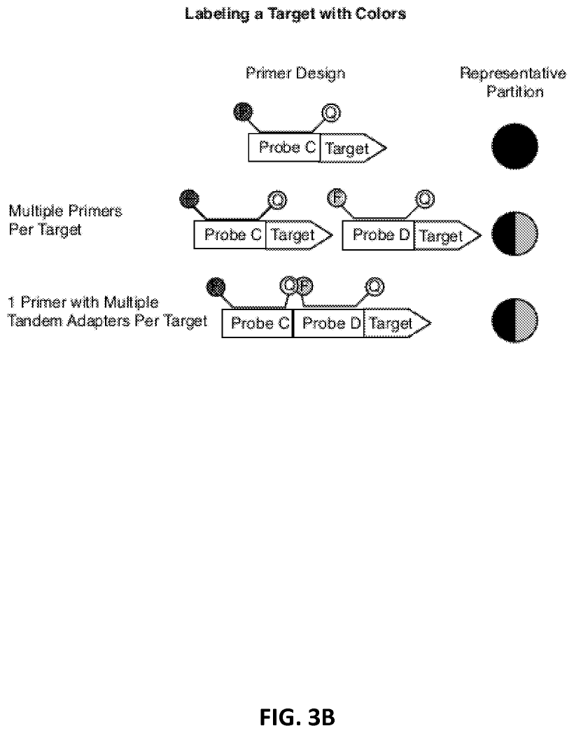

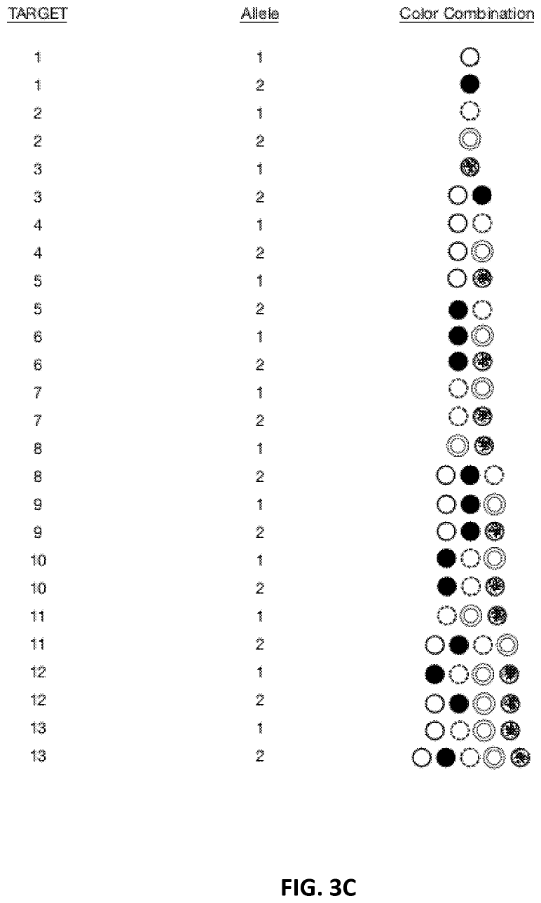

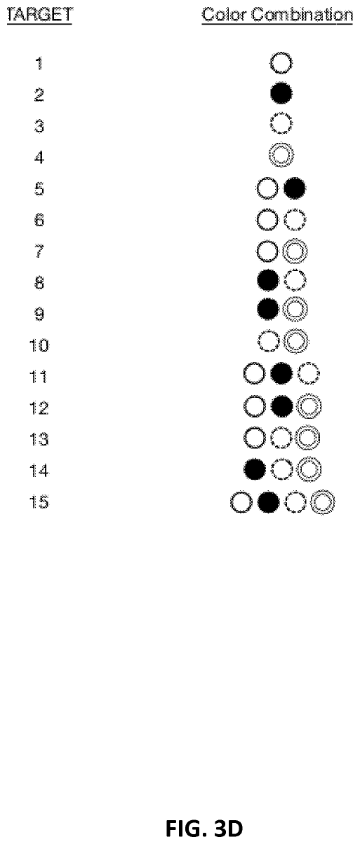

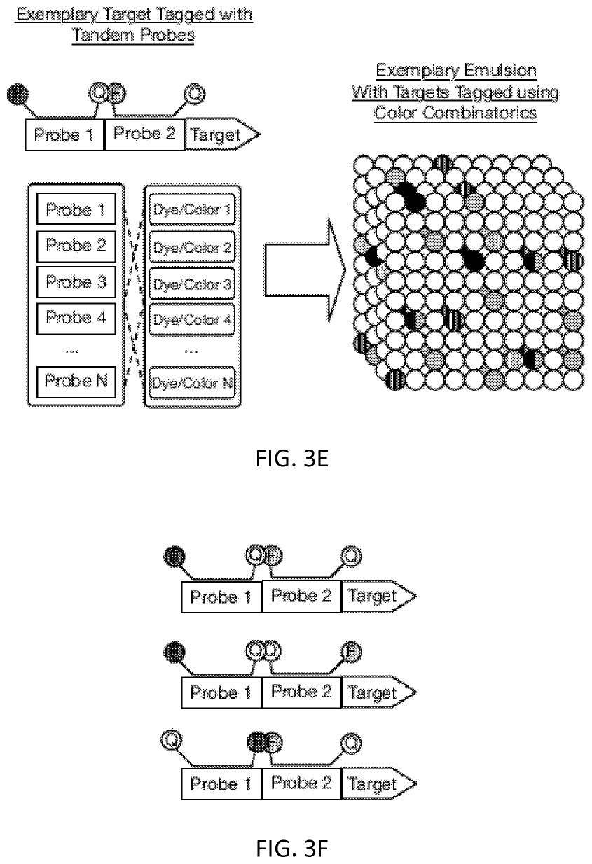

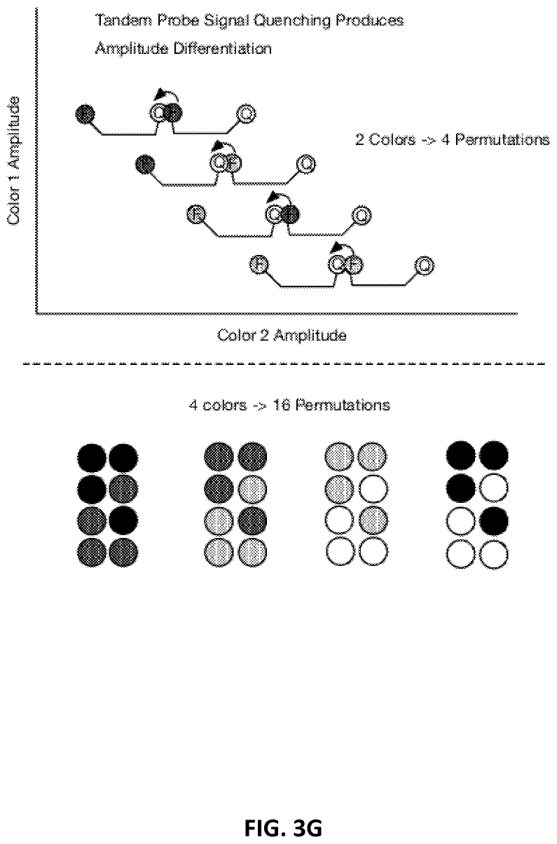

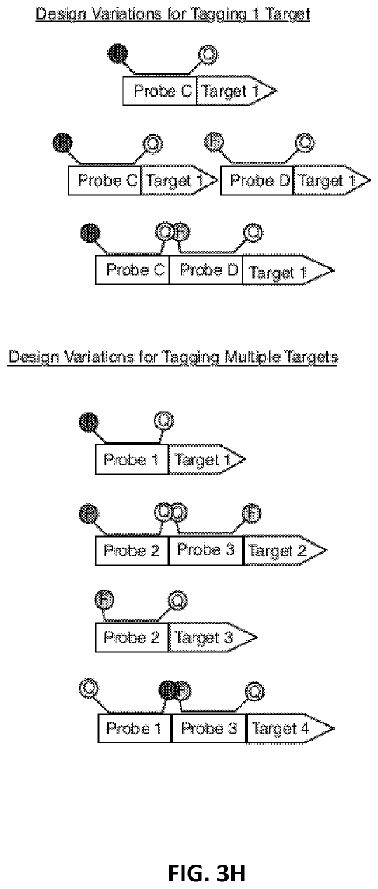

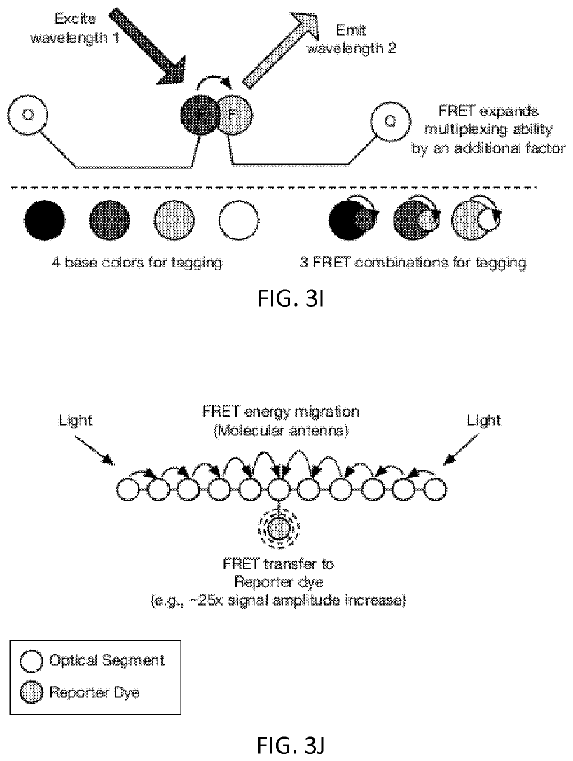

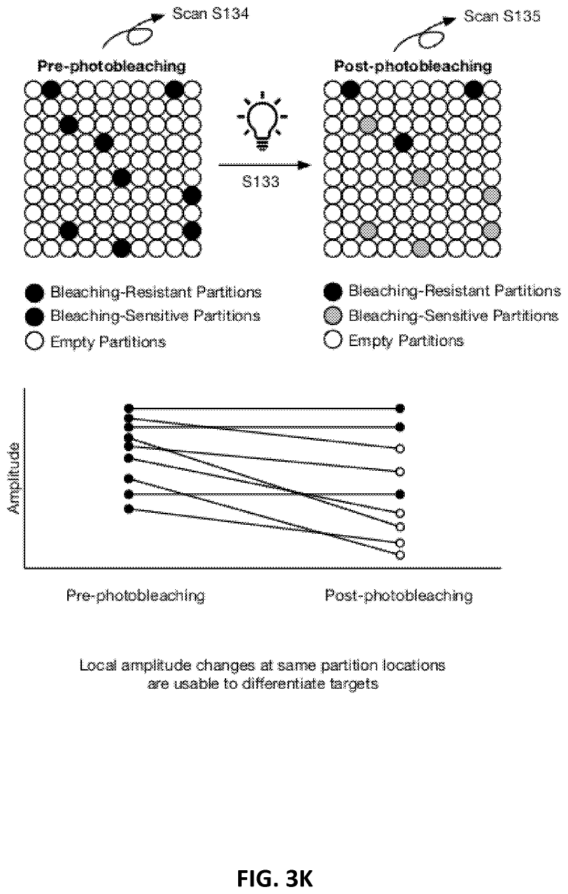

Multiplexing with color combinatorics is practically achievable using embodiments of the systems, methods, and processing materials described, especially due to the large number of partitions available (as described), and in some instances, low occupancy of partitions by targets (as described). For instance, as shown in FIG. 3A (left), low-partition systems may be subject to greater prevalence of multiple targets within a single partition (e.g., as in doublets, as in triplets), thereby contributing to a lower degree of multiplexed assay sensitivity. However, high-partition systems, also shown in FIG. 3A (right) may be subject to lower prevalence of multiple targets within a single partition (e.g., as in doublets, as in triplets), thereby contributing to a higher degree of multiplexed assay sensitivity when color combinatorics may be used. As such, a higher percentage of different targets that may be tagged with combinations of colors can be accurately discriminated with the high partition platform described. Table 1 below provides scenarios indicating doublet rates observable for systems with 20,000 partitions and 30,000,000 partitions (as in system embodiments described), respectively, where doublet rates may be provided for various inputs/counts per color channel used for scanning (and a significantly lower percentage of doubles may be observed with a high number of partitions).

TABLE 1

| | |
|---|---|
| 20K | 30M |
| Partitions | Partitions |

| | |
|---|---|
| Inputs/Counts | Doublet rate |

| | | |
|---|---|---|
| per channel | | |
| 1000 | 4.877% | 0.003% |
| 5000 | 22.120% | 0.017% |
| 10000 | 39.347% | 0.033% |
| 50000 | 91.792% | 0.167% |
| 100000 | 99.326% | 0.333% |
| 1000000 | — | 3.278% |

Furthermore, when implementing four or more colors for tagging a set of targets for multiplexed detection, the method 100 can include tagging targets with combinations of three or more colors, in order to reduce or otherwise eliminate error due to presence of doublets (i.e., two targets within a partition), where doublet targets, each tagged with single colors, may result in dual color partitions. Similarly, the method 100 can include tagging targets with combinations of four or more colors, in order to reduce or otherwise eliminate error due to presence of triplets (i.e., three targets within a partition), where triplet targets, each tagged with single colors, may result in tricolor partitions.

In relation to the set of processing materials described, labeling a target with multiple colors can be performed using multiple primers per target (e.g., gene), where each of a set of primers (e.g., forward primers) used to tag a respective target can tag the respective target with one of a set of dyes/fluorophores (2 primers/tandem probes are shown in representative FIG. 3B, but the methods can be adapted to more than 2 primers/tandem probes). Alternatively, labeling a target with multiple colors can be performed using a single primer (e.g., forward primer) for the target, along with tandem adapters (shown in FIG. 3B), where tandem adapters and probes are described in more detail in Section 2.1.3 below. Different tagging strategies (e.g., multiple primers per target vs. single primer per target) can be implemented. For instance, a set of primers (e.g., forward primers) can be used for tagging a first target with multiple colors, and a single primer (e.g., forward primer) can be used for tagging a second target with one or more colors. When using multiple primers, in silico primer design can be used to prevent undesired primer-primer, primer-probe, primer-amplicon (non-target) interactions and/or self-primer interactions (e.g., undesired hairpin structures, primer-dimer interactions, etc.) that may produce increased background during scanning.

In one case, in silico primer design can include generating multiple pairs of primers for individual targets of interest, with check steps to remove candidates with potential for amplicon/primer/probe interactions, to remove primers with multiple continuous matches (e.g., 11 or more continuous matches) to reduce cross-channel interactions in signal positive droplets and primer-probe interactions or background noise before amplification. Primer sets can be selected from the best primer pairs for each target, with secondary check steps for undesired primer-primer interactions and primer-amplicon interactions, removal of primers with multiple base pair (bp) continuous matches or 9 bp continuous matches in a region (e.g., last 10 bases). Such design steps can be used to create primer sets for which background is reduced for every partition (during use), and for which cross-channel interactions in positive droplets may be reduced. A resulting output of the in silico primer design operation produces a panel of compatible primers (e.g., with one primer pair per target of interest) for a specific multiplexing assay.

In relation to multiplexing with color combinatorics, labeling a target with multiple colors can be performed with combinations of colors, where the order of the colors used to tag a target is unaccounted for. Alternatively, labeling a target with multiple colors can be performed with permutations of colors, where the order of the colors used to tag a target is accounted for in relation to discrimination of a target. Permutation-based multiplexing is achievable using tandem probes used to tag targets, where tandem probes are described in more detail in Section 2.1.3 below. Implementation of fluorophores having transitionable fluorophore states (e.g., in response to a stimulus, in relation to exhibition of Foerster resonance energy transfer behavior, etc.) can also be used to achieve higher degrees of multiplexing, as described in more detail in Sections 2.1.3 and 2.1.4 below.

An example of targets (e.g., SNPs), corresponding alleles, and corresponding tagged-color combinations for detection and differentiation is shown in FIG. 3C, which enables encoding of 26 targets (e.g., 13 SNP loci) with a 5-color system (such that each partition can exhibit a color combinatoric of up to 5 colors). An example of targets (e.g., SNPs) and corresponding tagged-color combinations for detection and differentiation is shown in FIG. 3D, which enables encoding of 15 targets with a 4-color system (such that each partition can exhibit a color combinatoric of up to 4 colors). The example shown in FIGS. 3C and 3D can be adapted for systems greater than 5 colors or less than 4 colors. As such, with the ultra-high partition setting described involving limited dilution, each target of interest may be labeled with a unique set of colors for subsequent detection and digital quantitation. In some cases, the targets of interest may be labeled and may not comprise multiplexing based upon signal amplitudes.

Assays can be designed such that targets may be differentially tagged with combinations of colors in a manner that improves ability to discriminate color signals upon scanning and/or to use probes in a conservative and efficient manner. For instance, targets that may be anticipated to be most prevalent can be tagged with fewer colors, and targets that may be anticipated to be least prevalent can be tagged with more colors. Additionally or alternatively, non-similar targets (e.g., a first target and a second target that is non-similar to the first target) can be tagged with opposite color combinations, in order to improve the ability to discriminate non-similar targets upon scanning. For instance, in the context of microbiome analyses, bacteria-derived targets can be tagged with a first combination of colors and fungal targets can be tagged with a second combination of colors (e.g., colors that may be complementary to the first combination of colors, colors that may be near complementary to the first combination of colors, etc.) that allows for improved discrimination of bacterial targets and fungal targets upon scanning.

However, assay designs involving multiplexing with color combinatorics can be applied in another suitable manner.

2.1.3 Multiplexing with Tandem Probes, Conjugated Polymers, and Foerster Resonance Energy Transfer (FRET)

As noted above, higher degrees of multiplexing for partition-based systems can be achieved using tandem probes (e.g., a set of probes structured to tag the same target, for instance, with a single primer and tandem adapters for the target), such that each target being analyzed can be tagged with one or more of a set of probes (i.e., different probes configured to produce different color combinations of detectable signals). An example of a tandem probe design in shown in FIG. 3E, where individual probe sequences can be conjugated with one of a set of fluorophore/quencher combinations, and tagging a target with a subset of probes produces a signals that that can be detected with color channels appropriate to the subset of probes. As such, a target can be positively detected if signals corresponding to the subset of probes may be detected from a partition upon scanning the set of partitions with color channels corresponding to the subset of probes.

In variations, the fluorophores of the set of probes can all be positioned near a first end (e.g., 3′ end, 5′ end) of the respective probe, and the quenchers of the set of probes can all be positioned near a second end (e.g., 5′ end, 3′ end) of the respective probe, such that, as shown in FIG. 3F (top), the quencher of a first probe is positioned near the fluorophore of a second probe when the first probe and the second probe have tagged a target in tandem. Alternatively, as shown in FIG. 3F (middle), a first probe and a second probe to be used for tagging of a target in tandem can be configured such that the quencher of the first probe and the quencher of the second probe may be positioned near each other when the first probe and the second probe have tagged a target in tandem. Alternatively, as shown in FIG. 3F (bottom), a first probe and a second probe to be used for tagging of a target in tandem can be configured such that the quencher of the first probe and the quencher of the second probe may be positioned far from each other when the first probe and the second probe have tagged a target in tandem. However, in variations, the fluorophores and/or quenchers of probes can be positioned away from ends (e.g., 3′ ends, 5′ ends) of respective probes. Fluorophore and quencher positions along the lengths of respective probes can thus be configured to improve signal detection (e.g., in relation to detection of fluorescent signals from partitions/emulsion droplets, with desired quenching performance (e.g., with respect to quencher interference or quencher enhancement within a partition) and with desired background reduction performance).

While two tandem primers/tandem probes may be shown in FIG. 3H methods can be adapted and expanded to incorporate more than two primers/probes for target tagging. In particular, the methods described can involve up to 2 tandem probes for target tagging, up to 3 tandem probes for target tagging, up to 4 tandem probes for target tagging, up to 5 tandem probes for target tagging, up to 6 tandem probes for target tagging, up to 7 tandem probes for target tagging, up to 8 tandem probes for target tagging, up to 9 tandem probes for target tagging, up to 10 tandem probes for target tagging, or greater numbers of tandem probes for target tagging.

Additionally or alternatively, when the fluorophores of the set of probes may be positioned near a first end (e.g., 3′ end, 5′ end) of the respective probe, and the quenchers of the set of probes can all be positioned near a second end (e.g., 5′ end, 3′ end) of the respective probe, ordered positioning of a first probe with a first fluorophore and a first quencher and a second probe with a second fluorophore and a second quencher can be used to create different permutations of ordered probes that result in amplitude differentiation of signals for target detection. In particular, the effect of positioning quenchers and fluorophores in tandem and resulting signal intensity changes based upon positioning is due to FRET behavior, where, as a quencher of one probe is placed next to a fluorophore of another probe, the intensity of the fluorophore is reduced. Thus, by modulating relative positioning of quenchers and fluorophores of different probes used to tag a target, differential signal amplitudes for respective probes involved can be achieved and detected.

In the examples shown in FIG. 3G, when two fluorophore colors (i.e., a first color and a second color) may be available, tagging a target in tandem with a first probe having a first color and a second probe having the first color (FIG. 3G, top), enables detection of the target with a high amplitude signal corresponding to the first color. Tagging a target in tandem with a first probe having a first color and a second probe having a second color (FIG. 3G, second from top), enables detection of the target with a medium-high amplitude signal corresponding to the first color and a medium-low amplitude signal corresponding to the second color. Tagging a target in tandem with a first probe having a second color and a second probe having a first color (FIG. 3G, third from top), enables detection of the target with a medium-high amplitude signal corresponding to the second color and a medium-low amplitude signal corresponding to the first color. Tagging a target in tandem with a first probe having a second color and a second probe having a second color (FIG. 3G, bottom), enables detection of the target with a high amplitude signal corresponding to the second color. As such, when two colors may be available, 4 permutations of ordered pairs of tandem probes may be available for differential target tagging (with detectable signals based upon signal amplitude in different color channels corresponding to the colors available). When four colors may be available, 16 permutations of ordered pairs of tandem probes may be available (e.g., 4 permutations for each pair of colors), for differential target tagging, if two probes may be used per template. When four colors may be available, 256 permutations may be available (i.e., 4 probes with 4 colors×4 probes with 4 colors). Examples of signal amplitudes, for a scenario where four colors may be available and pairs of tandem probes may be used to tag targets, may be shown in FIG. 3G. Variations of the examples shown in FIG. 3G may not have the fluorophores and quenchers positioned at opposite ends of their respective probe, to provide differentiation of signals from targets using such tandem probes.

Additionally or alternatively, a first quencher can be added to a first probe used to tag a target, and based on the position of the first quencher of the first probe, the amplitude of a signal produced by a second fluorophore of a second probe used to tag a target in tandem with the first probe is reduced during detection. The amplitude reduction of the second fluorophore of the second probe can then enable discernment of the order of which the first probe (with the first fluorophore) and the second probe (with the second fluorophore) may be placed.

Additionally or alternatively, tandem probes can be configured to have a spacer region positioned between different probes used to tag the same target. The spacer region can reduce quenching effects provided by a quencher of a first probe and a fluorophore of a second probe positioned next to the first probe, where the length of the spacer modulates the quenching effect. As such, a longer spacer increases the resultant signal amplitude of a second probe spaced from the first probe by the spacer. Furthermore, resulting amplitudes associated with probes used can be modulated by tuning spacer length, in order to achieve additional granularity of amplitude levels of each dye used to tag one or more targets (and thus more permutations for multiplexing of targets). In variations, the spacer can have a length from 1 to 25 base pairs, and have a specific sequence or a random sequence, in relation to primer aspects described above.

In variations, as shown in FIG. 3H (left), a target (e.g., target gene) can be tagged using a target-specific primer (e.g., forward primer) with one or more detectable probes (e.g., Probe C and/or Probe D shown in FIG. 3H). As shown in FIG. 3H (right), multiple targets (e.g., target genes) can be tagged using primers (e.g., forward primers) with single probes and/or combinations of tandem probes, with signal detection for target identification from partitions performed as described. As such, tandem primer/probes and non-tandem primer/probes can be combined within the set of processing materials for tagging of different targets.

Alternatively, tandem probes where fluorophores may be positioned near each other (as shown in FIG. 3I) and capable of producing and/or responding a Foerster resonance energy transfer (FRET) effect (e.g., with one or more emitting fluorophores and one or more reporting fluorophores) can be used to enable signal detection based on FRET behavior. For instance, a first probe of a tandem probe can be excited by a first wavelength of light that matches the excitation spectrum of the first fluorophore, and FRET transfer to a reporter dye of a second probe can excite the second probe for detection of a target (e.g., using emission filter specific for the emission spectrum of the second fluorophore), thereby enabling differential detection of targets from different partitions given that the excitation wavelength profiles and the emission wavelength profiles for the set of partitions is identifiable with scanning. As such, in relation to a first fluorophore and a second fluorophore included in processing materials as described, embodiments of the method can further include causing Foerster resonance energy transfer (FRET) from the first fluorophore to the second fluorophore upon exciting the first fluorophore with a first wavelength profile of light, such that detecting signals from the set of partitions with the number of color channels can include detecting the target from a partition upon scanning the set of partitions with a second wavelength profile of light corresponding to the second fluorophore. As shown in FIG. 3I, a set of four fluorophores that exhibit FRET behavior in tandem with excitation of a cooler color first probe and FRET transfer and detection of a warmer color second probe, differential tagging and detection can be achieved for 7 targets.

As shown in FIG. 3J, the set of processing materials can additionally or alternatively include conjugated polymer probes for fluorescence enhancement, where such conjugated polymers operate as optical harvesters that exhibit long-range FRET capability. In more detail, as shown in FIG. 3J, a variation of a conjugated polymer probe includes a set of optical segments that harvest incident light with a large absorption cross-section. The structure of the conjugated polymer and set of optical segments determines the excitation wavelength. The set of optical segments operates as a molecular antenna and drive energy migration to a reporter dye of the probe, thereby achieving FRET transfer to the reporter dye.

Additionally or alternatively, conjugated polymer probes can include cationic polymers with complex structures (e.g., kinked structures, twisted structures, coiled structures, zigzagging structures, etc.) capable of FRET transfer from such complex structures to a reporter dye for amplification of signals produced by the reporter dye, with or without an emission spectrum shift by the reporter dye. As such, a subset number of excitation spectra (e.g., associated with a limited number of color channels) can be used to achieve color detection across more colors than the limited number of color channels. Dyes that may be included with or otherwise compatible with conjugated polymer probes include, for example, BD Horizon™ BB515, Alexa Fluor™ 488, FITC, BD Horizon™ BB630-P2, BD Horizon™ BB660-P2, PerCP-Cy5.5**, PerC**, BD Horizon™ BB700, BD Horizon™ BB755-P, BD Horizon™ BB790-P, and other BD Horizon™ components. Implementation of such conjugated polymer probes can, in some embodiments, be used to achieve detection of targets within partitions, with 12-color multiplexing, using 2 colors of light sources.

Additionally or alternatively, conjugated polymer probes can form complexes (e.g., electrostatic complexes) with quantum dots or other structures in a manner that can produce a cascading FRET effect, whereby energy is transferred to the quantum dots or other structures, from the conjugated polymer structure(s), upon being subject to excitation wavelengths of light, and energy is transferred from the quantum dots or other structures to the dye of the probe, in order to produce higher sensitivity.

Reporter dyes can include dyes discussed above and/or other dyes with distinct emission behavior, where FRET-based amplification of dyes can result in an orders of magnitude (e.g., one order of magnitude, two orders of magnitude, three orders of magnitude, etc.) or greater amplification effect in comparison to dyes without a conjugated polymer component.

### 2.1.4 Multiplexing Based Upon Stimulus-Responsive Dyes and Fluorophores

Additionally or alternatively, in the context of partitions that may be fixed in position or otherwise addressable (e.g., with barcoding, with identification of relative positions of partitions) across a set of scanning runs with the same color channel or different color channels, the method 100 can further include implementing stimulus-responsive dyes and/or fluorophores for tagging of targets, where scanning the set of partitions before and after applying a stimulus to the stimulus-responsive dyes and/or fluorophores enables additional levels of multiplexing to be achieved when using a limited number of color channels. As such, in relation to a base level of multiplexing achievable for a set of available color channels, the level of multiplexing can be expanded beyond the base level by a factor corresponding to the number of stimulus-responsive states available for each dye/fluorophore. For instance, multiplexing ability can be doubled (e.g., expanded by a factor of 2) for a color channel and use of a first fluorophore that is photobleaching resistant when irradiated using the color channel, and a second fluorophore that is photobleachable when irradiated using the color channel. Additional examples are provided below.

In variations, the dye(s)/fluorophore(s) can transition between states based upon application of a stimulus or stimuli, where the stimuli can involve one or more of: a stimulus involving irradiation, a stimulus involving a change in pH, a stimulus involving a change in temperature, a stimulus involving a change in pressure, a stimulus involving a change in electric field, a stimulus involving a change in magnetic field, or another suitable stimulus. Transition states of the dye(s)/fluorophore(s) can be binary (e.g., a first state and a second state) in response to an applied stimulus. Alternatively, a fluorophore can undergo transitions between a set of states (e.g., to different degrees), depending upon a method of application of the applied stimulus or stimuli, and/or number of same stimulus-response fluorophores attached to a target. For irradiation-responsive materials, stimulus application parameters can include intensity, wavelength, exposure duration, and other factors, and the amount of exposure to the stimulus can achieve different levels of photobleaching (which can have a more differentiable effect when multiple fluorophores may be used to tag the same target). For pH-responsive materials, stimulus application parameters can include pH value, temperature,
duration of exposure, and other factors and the amount of exposure to the stimulus can achieve different levels of photobleaching (which can have a more differentiable effect when multiple fluorophores may be used to tag the same target). For temperature-responsive materials, stimulus application parameters can include temperature, duration of exposure, and other factors and the amount of exposure to the stimulus can achieve different levels of photobleaching (which can have a more differentiable effect when multiple fluorophores may be used to tag the same target).

For multiplexing, the set of processing materials can include materials for tagging groups of dyes/fluorophores including one or more dyes/fluorophores that may be resistant to the stimulus (or may be not responsive to application of the stimuli) for a respective color channel, and one or more dyes/fluorophores that may be sensitive to the stimulus.

Examples of photostimulus-responsive dyes/fluorophores include Atto 495 (Blue color channel), FAM (Blue color channel), Bodipy-TMR (Green color channel), ROX (Yellow color channel), Cyanine 5 (Red color channel), Dy636 (Red color channel), Atto680 (Crimson color channel), and Cyanine 5.5 (Crimson color channel). Examples of photostimulus-resistant dyes/fluorophores include: Dy490 (Blue color channel), Atto488 (Blue color channel), Alexa488 (Blue color channel), Cyanine 3 (Green color channel), AttoRho 6G (Green color channel), HEX (Green color channel), VIC (Green color channel), SUN (Green color channel), Rhodamin 6G (Green color channel), Atto532 (Green color channel), Cyanin 3.5 (Yellow color channel), TEX615 (Orange color channel), Texas Red (Orange color channel), CAL Fluor 610 (Orange color channel), Bodipy-TR-X (Orange color channel), Atto590 (Orange color channel), Atto647N (Red color channel), and Dy682 (Crimson color channel).

Examples of pH sensitive fluorophores include pHrodo™ Green AM, pHrodo™ Red AM, fluorescein, LysoSensor Yellow, LysoSensor Blue, LysoSensor Green, Oregon Green 514, Oregon Green 488, Dichlorofluorescein derivatives, ACMA, HPTS, FAM, pHluorin, and pHluorin2.

Examples of temperature sensitive fluorophores include rhodamine B, Rhodamine 6G, Rhodamine C, Benzothiadiazoles, aza-BODIPY, phthalocyanines, perylene bisimide, and others.

Fluorophores or other colorimetric indicators can be differentially-responsive to the stimuli discussed, such that an applied stimulus produces differential responses in the fluorophores/colorimetric indicators. Alternatively, fluorophores or other colorimetric indicators can be equally-responsive or near-equally-responsive to an applied stimulus.

As such, implementation of the methods described can involve exposing tagged target analytes with stimulus-responsive tagging components, which can be differentially detectable before and/or after application of a stimulus, thereby increasing the level of plexy achievable with a limited number of color channels.

In relation to Step S130, the method 100 can additionally or alternatively include (for one or more color channels of a set of color channels involved in detection, and for a first fluorophore of the set of processing materials) use of a first fluorophore for achieving higher degrees of multiplexing, wherein the first fluorophore is a stimulus-responsive (e.g., photo-bleachable, photo-responsive, pH responsive, temperature-sensitive, etc.) fluorophore, and wherein detecting signals from the set of partitions comprises scanning the set of partitions prior to and post applying a stimulus to the first fluorophore, thereby transitioning the first fluorophore between a first state and a second state S133 (an example of which is shown in FIG. 3K). In a specific example, the applied stimulus is a light stimulus, and scanning can be performed with a first wavelength range of light to detect signals from the set of partitions prior to photobleaching, and after a second wavelength range of light configured to bleach the first fluorophore is applied. As such, the method 100 can include detecting signals from the set of partitions in a first phase of analysis upon scanning the set of partitions with the first wavelength range of light S134, and detecting signals from the set of partitions in a second phase of analysis upon scanning the set of partitions and bleaching the first fluorophore with the second wavelength range of light S135, as shown in FIG. 3K. As such, the set of processing materials described can include at least one primer structured to tag a first target with a first probe having a first fluorophore and a second target with a second probe having a second fluorophore, wherein the first fluorophore is a photo-bleachable fluorophore, thereby enabling differential detection of the first target and the second target. Alternatively, for Taqman™ chemistry, the set of processing materials can include, for a first target and a second target of the set of targets: a primer set comprising: a first primer structured to tag the first target with a first probe having a first fluorophore and a second primer structured to tag the second target with a second probe having a second fluorophore, wherein the first fluorophore is a photo-bleachable fluorophore, and wherein detecting signals from the set of partitions includes scanning the set of partitions with a first wavelength range of light and a second wavelength range of light configured to bleach the first fluorophore, thereby enabling differential detection of the first target and the second target.

Wavelengths of light used for scanning can be in the visible or non-visible spectrum. Light sources implemented can include laser light, light emitting diodes (LEDs), and/or other light sources. Laser powers implemented can include laser powers of 10 mW through 80 mW (or alternatively less than 10 mW or greater than 80 mW laser powers). However, other low power lasers can be implemented.

In a specific example, where the first fluorophore is FAM, scanning the set of partitions can include: scanning a set of planes of partitions within a collecting container, with a laser having a power of 50 mW and focused with optics to a 20 micron-thick light sheet (where other thicknesses less than 20 microns or greater than 20 microns can be implemented), where the duration of scanning for the set of planes (e.g., 500 planes) is at most 3 minutes, and wherein scanning bleaches the first fluorophore. Bleaching can be performed across a single scan of the set of planes, or across multiple scans of the set of planes. However, variations of the specific example can use other light sources (e.g., LEDs) that may be lower power, with longer exposure times to achieve bleaching or other states of signal emission characteristics. Detecting signals from the set of partitions can be performed prior to bleaching in a first characterization of the set of partitions, and post bleaching the first fluorophore for a second characterization of the set of partitions, in order to achieve higher degrees of multiplexing for differential detection of targets based upon the first characterization and the second characterization. Given that the locations of the partitions do not change (e.g., may be fixed in position within a stable emulsion), the first characterization can describe a first local signal amplitude for each droplet, and the second characterization can describe a second local signal amplitude for each droplet after the light stimulus is applied, where local amplitude characterizations can be achieved for partitions arranged in bulk with embodiments, variations, and examples of the platforms described.

Furthermore, scanning can be performed using different light wavelengths, powers, exposure times, and other parameters, for other transitionable fluorophores implemented in addition to the first fluorophore.

An example of signal discrimination pre and post photobleaching is shown in FIG. 3K.

Alternatively, for primarily non-photo responsive fluorophores, scanning the set of partitions can include: scanning a set of planes of partitions within a collecting container prior to application of a stimulus (e.g., temperature change, electric field, pH shift, mechanical stimulus, etc.); detecting signals from the set of planes of partitions for a first characterization of the set of partitions; scanning the set of planes of partitions within the collecting container post application of the stimulus (e.g., temperature change, electric field, pH shift, mechanical stimulus, etc.); detecting signals from the set of planes of partitions for a second characterization of the set of partitions; and characterizing targets of the sample in a multiplexed manner based upon the first characterization and the second characterization.

As such, in relation to other stimuli, the method 100 can include scanning the set of partitions with other wavelength(s) of light prior to and/or post-application of a stimulus (e.g., temperature change, electric field, pH shift, mechanical stimulus, etc.), where the stimulus causes changes in signal emission from the set of partitions appropriate to the wavelength(s) of light used for scanning.

2.1.5 Multiplexing with Different Levels of Plexy at Different Regions of a Density Gradient

In variations where the set of partitions comprise droplets of an emulsion, the emulsion can include subregions at different depths within the collecting container (e.g., vertical depths, radial depths, etc.), each subregion associated with a different level of plexy. For instance, a first subregion of the emulsion can include a first sample being assessed for targets of a first panel, and a second subregion of the emulsion can include a second sample being assessed for targets of a second panel, where the degree of multiplexing to characterize the first panel of targets of the first sample may be different from the degree of multiplexing that may be used to characterize the second panel of targets of the second sample.

In variations where the emulsion is generated upon applying a force (e.g., centrifugal force, pressure, etc.) to the sample(s) through a porous membrane or plate with openings, the first sample can have a first density and the second sample can have a second density, such that the first sample and the second sample self-arrange at different depths of the collecting container and have processing materials for different levels of plexy, at each depth of the collecting container. However, a gradient of regions can be formed in another suitable manner, in order to have different levels of assay plexy at different subregions of the collecting container, for different sets of targets of different samples.

### 2.1.6 Combined Multiplexing Options to Further Expand Levels of Plexy

In relation to the variations of multiplexing described (e.g., with color combinatorics, with stimulus-responsive materials, with density gradients involving layers of a container each having different degrees of plexy, etc.), multiplexing can be performed in a combinatorial manner, by implementing a number of different strategies, including color combinatorics, signal amplitude-based multiplexing (e.g., where discrimination of various targets is based upon signal amplitude with varied concentrations of primers, and when levels of background noise allow for accurate characterizations of signal amplitude corresponding to each target), stimulus-responsive fluorophores/dyes, different amplification and tagging chemistries (e.g., TaqMan-based chemistries described in more detail below, KASP-based chemistries, etc.) where a first level of plexy can be achieved with a first chemistry/assay design and a second level of plexy can be achieved with a second chemistry/assay design, chemistries involving Foerster resonance energy transfer (FRET) to produce a cascade of emission for different partitions, chemistries of the set of processing materials with non-hydrolysis probes, and other multiplexing technologies. For instance, chemistries with non-hydrolysis probes that may be capable of FRET behavior can be used to tag the set of targets with a set of permutations of the set of non-hydrolysis probes, where detecting signals from the set of partitions comprises detecting signals (e.g., based upon FRET from a first fluorophore of a first non-hydrolysis probe to a second fluorophore of a second non-hydrolysis probe) corresponding to the set of permutations, for differential detection of the set of targets. Additionally or alternatively, the set of processing materials can include a set of hydrolysis probes, such that the method includes tagging the set of targets with a set of combinations of the set of hydrolysis probes, and wherein detecting signals from the set of partitions includes detecting signals corresponding to the set of combinations for differential detection of the set of targets.

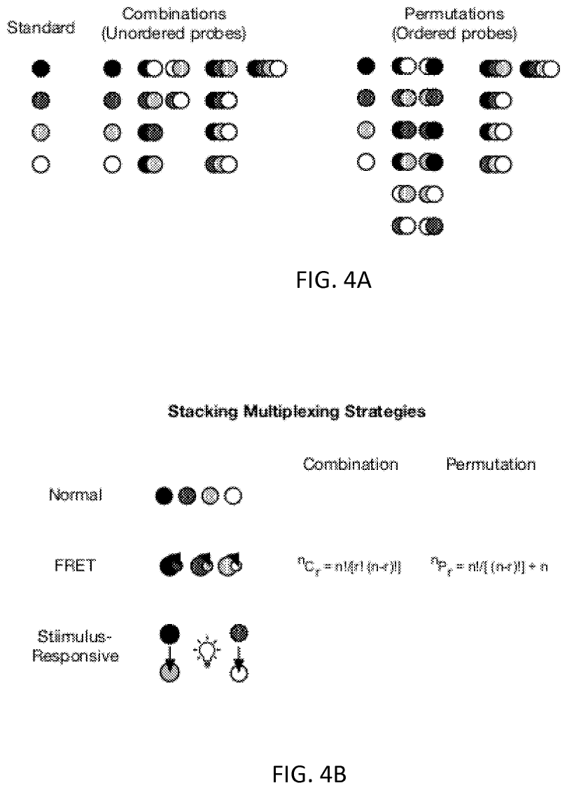

FIG. 4A shows the number of different targets that can be differentially tagged with four colors, without combinations or permutations of colors, with combinations of colors, and with permutations of colors. Without combinations or permutations of colors, four different targets can be differentially tagged and detected using four colors. With combinations four colors (with position-agnostic arrangements/orders of the colors used to tag the respective targets), 15 different targets can be differentially tagged and detected using four colors, and 10 different targets can be differentially tagged and detected using four colors if only up to two colors may be selected from the set of four colors. With permutations of four colors (with position-sensitive arrangements/orders of the colors used to tag the respective targets), 21 different targets can be differentially tagged and detected using four colors (with two colors of tandem probes), and 16 different targets can be differentially tagged and detected using four colors if up to two colors may be selected from the set of four colors.

As described above, the levels of multiplexing achieved can be enhanced (e.g., in an additive manner) with co-implementation of multiple multiplexing strategies and mechanisms. As shown in FIG. 4B, when combinations of colors may be used, the number of targets that can be differentially tagged can be represented by expression [1] below, where n represents the number of available colors, and r represents the number of selected colors from the number of available colors. When permutations of colors may be used, the number of targets that can be differentially tagged can be represented by expression [2], where n represents the number of available colors, and r represents the number of selected colors from the number of available colors.

$$nCr = n ! / [ r ! ( n ‐ r ) ! ] \qquad [ 1 ]$$

$$nPr = n ! / [ ( n - r ) ! ] + n \qquad [ 2 ]$$

Thus, with four available colors and up to three colors used in combination to tag targets, the number of targets that can be differentially tagged is 14. With four available colors and 2 colors used in permutation to tag targets, the number of targets that can be differentially tagged is 12.

When involving probes capable of FRET behavior, with four available base colors and 3 additional FRET-discriminable colors for a total of 7 signal types used in combination to tag targets, the number of targets that can be differentially tagged is 63 if up to 3 signal types may be selected and 28 if up to two signal types may be selected.

When additionally involving photobleachable probes, with four available base colors, 3 additional FRET-discriminable colors, and 2 photobleachable probes for a total of 9 signal types used in combination to tag targets, the number of targets that can be differentially tagged is 127 if up to 3 signal types may be selected and 45 if up to two signal types may be selected.

As such, the methods, systems, and compositions described can achieve extremely high levels of multiplexing even when a 3D imaging technique (e.g., light sheet imaging, 3D confocal microscopy, etc.) is involved for target detection, where background noise is much higher than for systems (e.g., microwell systems, etc.) where 1D and 2D imaging techniques may be sufficient for target detection.

### 2.1.7 Method—Assay Materials and Compositions Involving Allele-Targeting Probes

In alternative variations, the set of processing materials can utilize, for each target, a set of fluorophore-conjugated versions of the same probe sequence, where the set of fluorophore-conjugated versions produce a detectable combination of signals (e.g., color signals) that enable positive identification of the respective target. As such, the set of fluorophore-conjugated versions for each respective probe sequences can enable multiplexing based on color combinatorics to provide a high degree of multiplexing. In more detail, the set of processing materials can include a) for a respective target region of the set of targets, a forward primer, a reverse primer, and a set of fluorophore-conjugated probes, each of the set of fluorophore-conjugated probes targeting an allele of the respective target region and b) a master mixture including amplification reagents. The set of processing materials can additionally or alternatively include a probe additive for each of the set of fluorophore-conjugated probes, where the probe additive functions to quench background fluorescence resulting from the probes (i.e., is structured to reduce background noise), thereby improving signal-to-noise ratio (SNR). The set of fluorophore-conjugated probes can thus be configured to tag different alleles within a partition with different combinations of colors (corresponding to different fluorophores used), in order to provide discrimination of partition contents upon detection of signals from contents of each partition. A probe additive reagent can further include one or more quenches structured to interact with at least one of the 3′ region and the 5′ region of a fluorophore-labeled oligonucleotide.

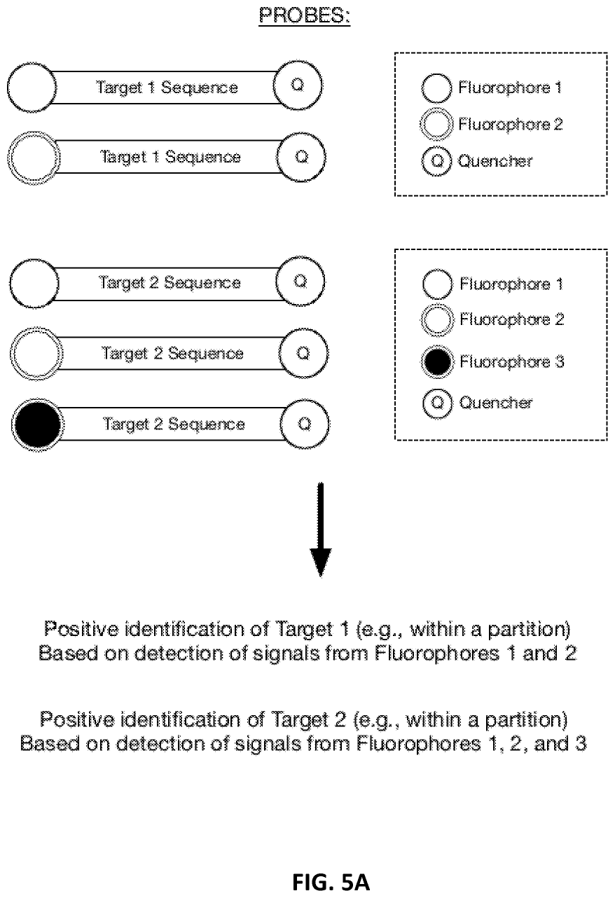

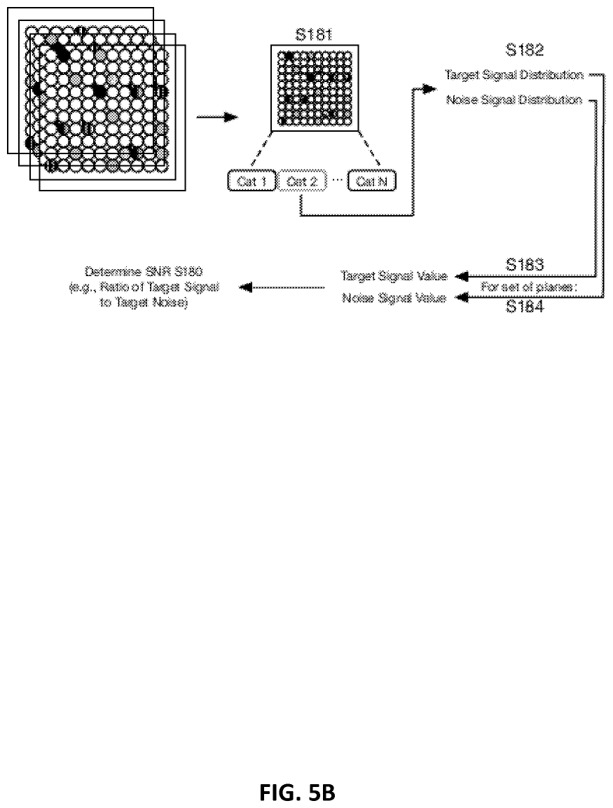

FIG. 5A depicts an example of fluorophore-conjugated probes that provide target detection and digital quantitation of different targets based on color combinatorics.

The probe additive can have a melting temperature (Tm) of 40-48° C. to be non-reacting during thermocycling (above 55° C. in general). However, in variations, the Tm of the probe additive can be greater than 48° C. or greater than 55° C. In examples the Tm of the probe additive can be from 47-79° C., greater than or equal to 79° C., or an intermediate value. In particular, with implementation of multiple and different probe additives, as primer diversity increases, primers can effectively compete with the probe additives to bind to respective probes, and some of the undesired primer-probe interactions cannot be eliminated via in silico design alone.

The set of probes can include Taqman™ probes and/or other dual-labeled probes to differentiate alleles of a target region. Probes can include dyes/fluorophores associated with chemical families including: acridine derivatives, arylmethine derivatives, anthracene derivatives, tetrapyrrole derivatives, xanthene derivatives, oxazine derivatives, dipyrromethene derivatives, cyanine derivatives, squaraine derivates, squaraine rotaxane derivatives, naphthalene derivatives, coumarin derivatives, oxadiazole derivatives, pyrene derivatives, and/or other chemicals. Such fluorophores can further be attached to other functional groups for tagging of targets in a detectable manner. Dyes/fluorophores can additionally or alternatively include compositions described in Section 2.1.1 above.

Primer concentration (e.g., forward primer concentration), probe concentration, and probe additive concentrations can be configured to improve detection performance (e.g., in relation to number of positive counts corresponding to positive targets, in relation to SNR values, etc.). In variations, the primer concentration (e.g., forward primer concentration): probe concentration: probe additive concentration can have a ratio of: 10:10:30; 10:20:60; 10:40:120; 10:80:240; 20:10:30; 20:20:30; 20:20:60; 20:40:120; 20:80:240; 40:10:30; 40:20:60; 40:40:120; 40:80:240; 80:10:30; 80:20:60; 80:40:120; 80:80:240; ratio values intermediate to those described; or other ratio values. Concentrations can be provided in terms of molarity or another suitable unit.

The example of targets (e.g., SNPs) and corresponding tagged-color combinations for detection and differentiation shown in FIGS. 3A and 3B can be similarly detected using the set of processing materials described here in Section 2.1.2. Similarly, the example shown in FIGS. 3C and 3D can be adapted for systems greater than 5 colors or less than 4 colors.

Quenchers of Taqman™ and/or other dual-label probes can be configured to quench signal of the fluorophore if the quencher is in proximity to the fluorophore below a threshold distance). Additionally or alternatively, quenchers can include one or more of: black hole quenchers, static quenchers, self-quenchers (e.g., fluorophores that self-quench under certain conditions by producing secondary structures or other structures), and/or other suitable quenchers. Quenchers can be used to suppress background signals (e.g., for 3D imaging applications, for other detection applications).

However, the set of processing materials can additionally or alternatively include other suitable components and/or be configured in another suitable manner.

2.2 Method—Partitioning of Sample with Processing Materials

Distributing the sample combined with the set of processing materials, across a set of partitions in step S140 can include receiving a sample (variations and examples of which may be described above) at a vessel passively or actively (e.g., with applied force, such as with gravitational force, with centrifugal force, with pressurization, etc.). The sample and processing materials can be delivered manually (e.g., with a fluid aspiration and delivery device, such as a pipettor). The sample and processing materials can additionally or alternatively be delivered with automation (e.g., using liquid handling apparatus or other sample handling apparatus).

In variations, vessel formats can include tubes (e.g., PCR tubes) containing partitions of the sample (e.g., in partition network format, in permeable partition network format, in gel-format, in emulsion format, in another format), wells (e.g., microwells, nanowells, etc.), channels, chambers, and/or other suitable containers. Additionally or alternatively, alternative variations of step S130 can include receiving the sample at other suitable substrates (e.g., slides, plates, etc.) functionalized with material components structured to interact with target material of the sample. For instance, sample material can be spotted onto substrates with material components structured to interact with target material of the sample and in a detectable manner.

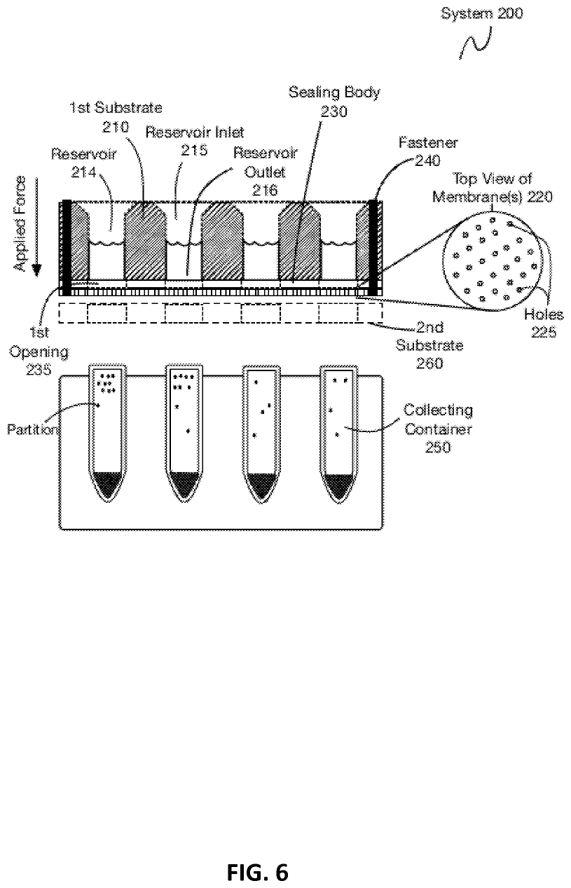

Embodiments, variations, and examples of the methods described can be implemented by or by way of embodiments, variations, and examples of components of system 200 shown in FIG. 6, with a first substrate 210 defining a set of reservoirs 214 (for carrying sample/mixtures for droplet generation), each having a reservoir inlet 215 and a reservoir outlet 216; one or more membranes (or alternatively, droplet-generating substrates) 220 positioned adjacent to reservoir outlets of the set of reservoirs 214, each of the one or more membranes 220 including a distribution of holes 225; and optionally, a sealing body 230 positioned adjacent to the one or more membranes 120 and including a set of openings 235 aligned with the set of reservoirs 214; and optionally, one or more fasteners (including fastener 240) configured to retain the first substrate 210, the one or more membranes 220, and optional sealing body 230 in position relative to a set of collecting containers 250. In variations, the system 200 can additionally include a second substrate 260, wherein the one or more membranes 220 and optionally, the sealing body 230, may be retained in position between the first substrate 210 and the second substrate 260 by the one or more fasteners. In using embodiments, variations, and examples of the system 200, material derived from each sample is retained in its own tube and may not comprise batching and pooling, allowing for scalable batch size.

In variations, the distribution of holes 225 can be generated in bulk material with specified hole diameter(s), hole depth(s) (e.g., in relation to membrane thickness), aspect ratio(s), hole density, and hole orientation, where, in combination with fluid parameters, the structure of the membrane can achieve desired flow rate characteristics, with reduced or eliminated polydispersity and merging, suitable stresses (e.g., shear stresses) that do not compromise the single cells but allow for partitioning of the single cells, and steady formation of droplets (e.g., without jetting of fluid from holes of the membrane).

In variations, the hole diameter can range from 0.02 micrometers to 30 micrometers, and in examples, the holes can have an average hole diameter of 0.02 micrometers, 0.04 micrometers, 0.06 micrometers, 0.08 micrometers, 0.1 micrometers, 0.5 micrometers, 1 micrometers, 2 micrometers, 3 micrometers, 4 micrometers, 5 micrometers, 6 micrometers, 7 micrometers, 8 micrometers, 9 micrometers, 10 micrometers, 20 micrometers, 30 micrometers, any intermediate value, or greater than 30 micrometers (e.g., with use of membrane having a thickness greater than or otherwise contributing to a hole depth greater than 100 micrometers).

In variations, the hole depth can range from 1 micrometer to 200 micrometers (e.g., in relation to thickness of the membrane layer) or greater, and in examples the hole depth (e.g., as governed by membrane thickness) can be 1 micrometers, 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or any intermediate value.

In variations, the hole aspect ratio can range from 5:1 to 200:1, and in examples, the hole aspect ratio can be 5:1, 10:1, 20:1, 30:1, 40:1, 50:1, 60:1, 70:1, 80:1, 90:1, 100:1, 125:1, 150:1, 175:1, 200:1, or any intermediate value.

In variations, the hole-to-hole spacing can range from 5 micrometers to 200 micrometers or greater, and in examples, the hole-to-hole spacing is 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or greater. In a specific example, the hole-to-hole spacing is greater than 10 micrometers.

In examples, the hole orientation can be substantially vertical (e.g., during use in relation to a predominant gravitational force), otherwise aligned with a direction of applied force through the distribution of holes, or at another suitable angle relative to a reference plane of the membrane or other droplet generating substrate 120.

Additionally or alternatively, embodiments, variations, and examples of the methods described can be implemented by or by way of embodiments, variations, and examples of components described in U.S. application Ser. No. 17/687,080 filed 4 Mar. 2022, U.S. Pat. No. 11,242,558 granted 8 Feb. 2022, U.S. application Ser. No. 16/309,093 filed 25 May 2017, and PCT Application PCT/CN2019/093241 filed 27 Jun. 2019, each of which is herein incorporated in its entirety by this reference. However, methods described can additionally or alternatively implement other system elements for sample reception and processing.

### 2.3 Method—Target-Specific Tagging and Amplification

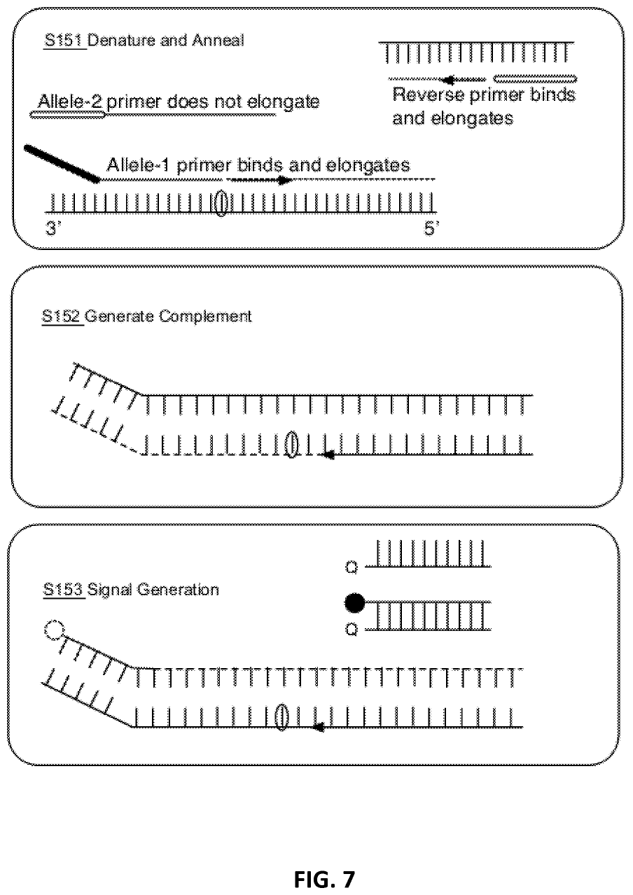

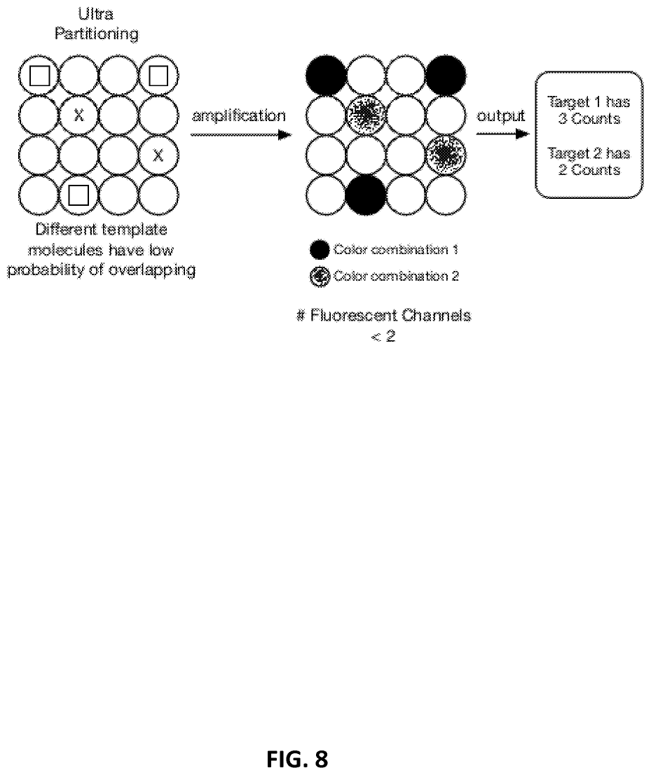

Step S150 recites performing target-specific (e.g., allele-specific) tagging and amplification (and/or providing suitable environments for supporting such operations), with the set of processing materials, for target regions associated with the set of targets across a set of stages. Step S150 and associated Steps S151, S152, and S153 (described in more detail below and in shown in the schematic of FIG. 7 and FIG. 8) may occur within partitions generated from the sample.

In one embodiment, the sample can be combined with an embodiment, variation, or example of the set of processing materials described above, and then partitioned such that template molecules of the sample occupy individual partitions with minimal overlap between different template molecules. Partitioning can be performed by passing the sample combined with the set of processing materials through a partitioning device (e.g., to generate droplets, to generate an emulsion with droplets provided in a continuous phase, etc.). Partitioning can alternatively be performed by distribution of the sample combined with the set of processing materials across a set of containers (e.g., microwells, nanowells, etc.). Partitioning can still alternatively be performed by distributing the sample combined with the set of processing materials across a substrate (e.g., as spots) and/or in another suitable manner.

As shown in FIG. 7, Step S150 functions to denature the template material (e.g., DNA template) of the sample, anneal components to the target region(s) being evaluated, and amplify the target region(s) in a first stage, with generation of complements of allele-specific tail sequences in a second stage. Then, in subsequent stages of amplification, amounts of allele-specific tagged sequences increase in a manner that can be detected (e.g., through an optical-based detection method).

As shown in FIG. 7, the first stage of sample processing S151 can include denaturing of sample material (e.g., sample DNA) and processing the denatured sample material with primers (i.e., allele-specific forward primers grouped with corresponding reverse primers for each target). In the first stage, one of the allele-specific forward primers of the set of sample processing materials matches the target (e.g., target SNP) and, with the common reverse primer, amplifies the target region. As such, targets (e.g., target SNPs) present in the sample may be amplified upon interacting with respective allele-specific forward primers.

As shown in FIG. 7, the second stage of sample processing S152 can include generation of allele-specific sequences (e.g., tail sequences), where the common reverse primer binds to, elongates, and produces a complimentary copy of a labeled tail sequence corresponding to the target allele.

As shown in FIG. 7, the third stage of sample processing S153 and subsequent stages can include one or more rounds of amplification/PCR to produce a detectable signal, whereby levels of tagged allele-specific sequences increase until a detection threshold is reached and/or surpassed. In the third stage S153 and subsequent stages of sample processing, labeled oligonucleotides bind to new complementary sequences (e.g., tail sequences), releasing fluorophores from corresponding quenchers to produce detectable signals for each target (e.g., target SNP, other target) present in the sample. However, fluorophores corresponding to targets that may be not present may be not released and thus continue to be quenched during rounds of amplification. In particular, with regard to parameters associated with threshold cycles at which or beyond which amplified targets become detectable (e.g., Ct, Cp, Cq, etc.), step S153 can include detecting and/or returning results indicative of target presence prior to the end-point of the process and/or at the end-point of the process (e.g., as in end-point PCR). Additionally or alternatively, real-time measurement of signals can be performed contemporaneously with each cycle of amplification.

In relation to the one or more stages of sample processing, activation-associated steps can be performed at a temperature or temperature profile (e.g., 90° C., 92° C., 94° C., another suitable temperature), for a duration of time (e.g., 10 minutes, 12 minutes, 15 minutes, another suitable duration of time), and/or for a number of cycles (e.g., 1 cycle, 2 cycles, another suitable number of cycles). In relation to the one or more stages of sample processing, denaturation-associated steps can be performed at a temperature (e.g., 90° C., 92° C., 94° C., another suitable temperature) or temperature profile, for a duration of time (e.g., 10 seconds, 15 seconds, 20 seconds, 25 seconds, another suitable duration of time), and/or for a number of cycles (e.g., 1 cycle, 5 cycles, 10 cycles, 20 cycles, another suitable number of cycles). In relation to the one or more stages of sample processing, annealing/elongation-associated steps can be performed at a temperature or temperature profile (e.g., 52-70° C. with a ramp down rate, another suitable temperature profile), for a duration of time (e.g., 20 seconds, 30 seconds, 60 seconds, 90 seconds, another suitable duration of time), and/or for a number of cycles (e.g., 1 cycle, 5 cycles, 10 cycles, 20 cycles, 25 cycles, 30 cycles, another suitable number of cycles).

In a specific example, activation-associated steps in a first stage of sample processing can be performed at a temperature of 94° C., for 15 minutes, with 1 cycle. In the specific example, denaturation-associated steps in a second stage of processing can be performed at 94° C. for 20 seconds, with annealing/elongation performed from 61-55° C. (with a drop of 0.6° C./cycle), for 60 seconds and for 10 cycles. In the specific example, denaturation-associated steps in a third stage of processing can be performed at 94° C. for 20 seconds, with annealing/elongation performed at 55° C. for 60 seconds and for 26 cycles. Additional denaturation-associated steps can be performed at 94° C. for 20 seconds, with annealing/elongation performed at 57° C. for a suitable number of cycles (e.g., 3 cycles).

In another specific example, activation-associated steps in a first stage of sample processing can be performed at a temperature of 94° C., for 15 minutes, with 1 cycle. In the specific example, denaturation-associated steps in a second stage of processing can be performed at 94° C. for 20 seconds, with annealing/elongation performed from 65-57° C. (with a drop of 0.8° C./cycle), for 60 seconds and for 10 cycles. In the specific example, denaturation-associated steps in a third stage of processing can be performed at 94° C. for 20 seconds, with annealing/elongation performed at 57° C. for 60 seconds and for 30 cycles. Additional denaturation-associated steps can be performed at 94° C. for 20 seconds, with annealing/elongation performed at 57° C. for a suitable number of cycles (e.g., 3 cycles).

Stages of sample processing in Block S150 can further include implementation of additives (described in Section 2 above) to improve signal-to-noise ratio (SNR) characteristics in the context of multiplexed detection, by increasing signal characteristics and/or reducing background (e.g., noise other artifacts). Additionally or alternatively, stages of sample processing in Step S150 can implement other components (e.g., density gradient mediums) to improve SNR.

In particular, in the context of emulsion digital PCR with the numbers of partitions described, such multiplexed assay design aspects described can produce significantly improved signal-to-noise (SNR) values with reduced background, in relation to detection techniques described below (e.g., based on light sheet imaging, etc.). In examples, target signals can be at least 102 greater than background noise signals, 103 greater than background noise signals, 104 greater than background noise signals, 105 greater than background noise signals, 106 greater than background noise signals, 107 greater than background noise signals, or better. Background noise can be attributed to fluorescence from adjacent partitions and adjacent planes of the set of planes of partitions in the context of emulsion digital PCR, or attributed to other sources with closely-positioned partitions. Determining the SNR can include scanning a set of planes of the set of partitions, determining a target signal value and a noise signal value for the set of planes, and determining the SNR from the target signal value and the noise signal value S180, where a variation of determining the target signal value and the noise signal value is described below.

In examples associated with reaction materials described and used for droplet digital PCR, determining the target signal value and a noise signal value can include: for each plane of a set of planes of partitions under interrogation (e.g., by light sheet detection, fluorescent microscopy, confocal microscopy, detection by photodiodes, by another method of detection, etc.): determining a categorization (of a set of categorizations for the respective plane) based upon a profile of signal-positive partitions represented in a respective plane S181, determining a target signal distribution and a noise signal distribution specific to the profile S182. Here, a target signal value can be determined from the target signal distribution S183, and can be an average value (or other representative value) of the target signal intensities determined from the set of planes. Similarly, a noise signal value can be determined from the noise signal distribution S184, and the background noise signal value can be an average value (or other representative value) of the noise signal intensities determined from the set of planes. A schematic is shown in FIG. 5B.

However, materials used for the amplification and/or detection reactions can be otherwise configured to improve SNR.

### 2.4 Method—Signal Detection and Returned Outputs

As shown in FIG. 1A, the method 100 can include Step S110, which recites detecting signals indicative of a profile of a set of targets, from a processed sample (e.g., within a single vessel, within a set of vessels) S110. Step S110 functions to enable detection of signals from dyes/fluorophores that may be released upon processing the sample with the set of processing materials, thereby providing indications of presence of targets (e.g., SNP targets, other targets) within the sample. In particular, with regard to parameters associated with threshold cycles at which or beyond which amplified targets become detectable (e.g., Ct, Cp, Cq, etc.), step S110 can include detecting and/or returning results indicative of target presence prior to the end-point of the process and/or at the end-point of the process (e.g., as in end-point PCR). Additionally or alternatively, real-time measurement of signals can be performed contemporaneously with each cycle of amplification.

Processed sample material can include samples processed according to methods described above, with respect to multiplexed tagging of alleles of targets of interest.

In variations, detection of signals can include irradiating processed sample material with suitable excitation wavelengths of light, and/or receiving emitted wavelengths of light corresponding to released dyes/fluorophores. As such detection of signals can be implemented by an optical signal detection subsystem (e.g., imaging subsystem), including embodiments, variations, and examples of systems described above. In particular, detection subsystems can be structured for detection of signals from partitions (e.g., by light sheet imaging, by fluorescence microscopy, by confocal microscopy, by another suitable optical detection subsystem, etc.) using combinations of filters and/or color channels, where signals from individual partitions may be detected in a high-partition number but low-occupancy regime. As such, detection can be performed for partitions arranged in 3D (e.g., as in droplets of an emulsion within a container), in 2D (e.g., for a monolayer or bi-layer of partitions at a substrate), and/or in another suitable format.

In variations, the sample can be processed with the set of processing materials in coordination with distribution of the sample across a set of partitions, where the set of partitions can include droplets (e.g., droplets of an emulsion, droplets provided in a sheathing fluid, gel droplets, other forms of droplets), microchambers, microwells, spotted samples on a substrate, and/or other partitions. As such, the partitions can be provided within one or more of: a container configured for centrifugation (e.g., a centrifuge tube, a microcentrifuge tube, etc.), a process container for PCR (e.g., a PCR tube), a strip tube, a plate having wells (e.g., a microtiter plate, a multi-well plate, a microwell plate, a nanowell plate, etc.), or another suitable collecting container. Additionally or alternatively, partitions can include regions of sample provided in another manner upon a substrate (e.g., spotted onto a substrate/slide).

With respect to sample processing using the set of processing materials, reactions within individual partitions can thus produce signals that may be detected by systems that can detect signals from multiple partitions or all partitions simultaneously in a distinguishable manner (e.g., with a 3D scanning technique, as described above). Alternatively, reactions within individual partitions can produce signals that may be detected by systems that can detect signals from individual partitions in a sequential manner.

Characterizing a set of targets of a sample (e.g., in relation to presence/absence, in relation to counts of targets), upon scanning the set of partitions, can include: generating a multivariable vector of emission values (e.g., emitted intensity values across the set of available color channels), for each detected emitted signal from a respective partition, paired with the excitation parameters used to excite the set of partitions (e.g., in the context of probes that exhibit FRET and/or probes that can be photobleached); performing a clustering operation with the multivariable vectors of emission values generated from the set of partitions; sorting partitions of the set of partitions into a set of categories corresponding to targets of the set of targets, based upon the clustering operation and a set of probes used to tag the set of targets; and generating a count of each of the set of targets represented in the set of partitions, based upon said sorting. In variations, the clustering operation can include performance of a co-localization operation, whereby scanning deviations may be corrected for in order to further delineate/provide stronger discrimination between different clusters of partitions. Additionally or alternatively, clustering algorithms can further include one or more of: principal component analysis (PCA), k-means clustering, t-stochastic neighbor embedding (t-SNE), UMAP clustering, and/or other algorithms. In variations, characterizing partitions can further include identifying partitions that may be signal positive in more than one channel, in relation to color combinatorics described above.

Step S120 recites returning a characterization of the processed sample based upon the profile. Step S120 functions to provide information pertaining to presence or absence of the set of targets associated with the sample being evaluated, and/or presence or absence of variants of the set of targets. The characterization can then be used to provide diagnostics and/or to support diagnostics of the organism(s) from which the processed sample was sourced, and/or to provide quality for conclusiveness of diagnostic results. Additionally or alternatively, the characterization can be used to guide provision of therapeutics (e.g., personalized therapeutics) corresponding to determined states of the organism(s) from which the processed sample was sourced, in order to improve or maintain health statuses of the organism(s).

In specific applications, the characterization can be used to inform diagnostics, provide other characterizations (e.g., of disease resistance, of disease predisposition, of genetic relationships, etc.) and/or guide generation of therapeutics associated with non-invasive prenatal testing (described above and in more detail below). More broadly, outputs of step S120 can be used to characterize (e.g., based on relative abundance measurements) self genetic material (e.g., genetic material of an organism) and non-self genetic material (e.g., genetic material not of the organism, genetic material of a different organism) from a sample.

Additionally or alternatively, in specific applications, the characterization can be used to inform characterizations of a subject from which the sample is sourced, in relation to one or more of: cancers, integumentary system conditions, skeletal system conditions, muscular system conditions, lymphatic system conditions, respiratory system conditions, digestive system conditions, nervous system conditions, endocrine system conditions, cardiovascular system conditions, urinary system conditions, reproductive system conditions, and/or other conditions.

Outputs can additionally or alternatively support at least one of: pathogen detection, non-invasive prenatal testing, organ transplantation analysis, forensics, and oncology, based upon the quantitative analysis.

In other specific applications, the characterization can be used to inform diagnostics, provide other characterizations (e.g., of disease resistance, of disease predisposition, of genetic relationships, etc.) and/or guide generation of therapeutics in the context of other multicellular organisms, plants, fungi, unicellular organisms, viruses, and/or other subjects.

### 2.5 Method—Signal Detection and Returned Outputs for Fetal Fraction and Non-Invasive Prenatal Testing Applications

As shown in FIG. 1C, in a variation, a method 100b for determination of fetal fraction (FF) can include Step S110c, which recites detecting signals indicative of a profile of a set of single nucleotide polymorphisms from a sample. Step S110c functions to enable characterizations of presence or absence of alleles of a set of SNPs from a sample (e.g., a maternal sample, other sample), which can be used to determine FF and inform conclusiveness of results in NIPT applications. In particular, with regard to parameters associated with threshold cycles at which or beyond which amplified targets become detectable (e.g., Ct, Cp, Cq, etc.), step S110c can include detecting and/or returning results indicative of SNP allele presence prior to the end-point of the process and/or at the end-point of the process (e.g., as in end-point PCR).

Processed sample material can include samples processed according to methods described above, with respect to multiplexed tagging of alleles of SNPs of interest.

In variations, detection of signals can include irradiating processed sample material with suitable excitation wavelengths of light, and/or receiving emitted wavelengths of light corresponding to released dyes/fluorophores. As such detection of signals can be implemented by an optical signal detection subsystem (e.g., imaging subsystem), including embodiments, variations, and examples of systems described above. In particular, detection subsystems can be structured for detection of signals from partitions (e.g., by light sheet imaging, by another suitable optical detection subsystem, etc.) using combinations of filters and/or color channels, where signals from partitions may be detected in a high-partition number but low-occupancy regime.

In variations, the sample can be processed with the set of processing materials in coordination with distribution of the sample across a set of partitions, where the set of partitions can include droplets (e.g., droplets of an emulsion, droplets provided in a sheathing fluid, gel droplets, other forms of droplets). Additionally or alternatively, the partitions can be provided within one or more of: a container configured for centrifugation (e.g., a centrifuge tube, a microcentrifuge tube, etc.), a process container for PCR (e.g., a PCR tube), a strip tube, a plate having wells (e.g., a microtiter plate, a multi-well plate, a microwell plate, a nanowell plate, etc.), or another suitable collecting container. Additionally or alternatively, partitions can include regions of sample provided in another manner upon a substrate (e.g., spotted onto a substrate/slide).

Step S120c recites returning a characterization of relative abundance of alleles of each SNP in the set of SNPs to generate an estimate of fetal DNA fraction in the sample, which functions to enable determinations of conclusiveness of NIPT results.

In variations, SNP alleles processed and evaluated in a massively parallel manner to determine FF in step S120c can include SNPs associated with chromosomes 1, 13, 18, 21, X, and/or Y, at various loci (e.g., from 10 to 20,000 polymorphic loci); however, SNPs characterized to determine FF can additionally or alternatively be associated with other chromosomes and/or loci. SNPs evaluated can be biallelic or multiallelic, with more than two alleles per SNP. SNPs evaluated can further be characterized by a high minor allele fraction (MAF), with an MAF above a suitable threshold (e.g., MAF>0.2, MAF>0.3, MAF>0.4, etc.); however, SNPs evaluated can be characterized with other MAF values. SNPs evaluated can be for coding regions (e.g., synonymous, non-synonymous, missense, nonsense) and/or non-coding regions.

With respect to determination of FF in Step S120c, target panels undergoing evaluation can be designed such that FF associated with fetus of any gender can be determined, without detection of chromosome Y markers. As such, for a male fetus, FF can be estimated by the amount of chromosome Y fragments present in the sample (e.g., maternal sample) relative to the amount of other non-sex chromosomes. For determination of FF for a female fetus, the set of SNPs evaluated may be selected such that for each fetus-mother pair, there may be at least a few SNPs in the common SNP panel that may be homozygous in mother and heterozygous in fetus. The count of the alternate allele from the fetus, when compared to the count of the homozygous allele (from mother, and also half from fetus), may yield FF for a female fetus (or non-male fetus, such as in intersex conditions).

In a specific application, the method can implement counting rules per reference chromosome to provide indications of confidence in NIPT assay results with respect to threshold FF values. In a specific example, for a counting requirement of 400,000 counts per reference chromosome, the lowest FF (e.g., DNA FF) in which an aneuploidy assay may be confident in calling a true negative is ~4%; thus, the FF assay estimates <4% DNA FF, then the results from the aneuploidy assay may be inconclusive. However, if the FF assay estimates >4% DNA FF, then the results from the aneuploidy assay may be more conclusive with increasing FF.

However, in other specific examples, the counting requirement per reference chromosome can be set at another value (e.g., less than 400,000 counts, greater than 400,000 counts, etc.) in relation to other FF threshold values (e.g., 3%, 5%, 6%, other percentages, etc.).

Expansions of the methods can be applied to detection of sex aneuploidies (e.g., Klinefelter syndrome, Turner syndrome, etc.), trisomies (e.g., Downs syndrome, Edwards syndrome, Palau syndrome, etc.), and/or other genetic conditions.

### 2.6 Method—Signal Detection and Returned Outputs for Transplant Rejection Applications

In another example, materials and methods described can be adapted for characterization and/or early detection of transplant rejection in a subject. Methods described here in Section 2.6 can be used to detect and digitally quantify donor-specific genetic material (e.g., dd-cfDNA, ds-DNA, GcfDNA) in a sample from a subject who has received a transplant, such that the sample potentially contains a quantifiable amount of self genetic material and donor genetic material. Furthermore, longitudinal characterization of the amount of donor genetic material in samples acquired from the subject at different time points can be used to assess onset of transplant rejection, where increases in donor genetic material over time can serve as a proxy for transplant rejection.

The biological rationale behind the utility of donor genetic material as a biomarker for transplant rejection is that the immune system of the subject receiving a transplant is activated upon recognition of the transplanted material (e.g., organ, cells, etc.) and produces antibodies in response. The antibodies attack the transplanted material, which leads to apoptosis or cell necrosis. The ruptured or dead cells then release their contents into the subject's blood plasma and thereafter, the subject carries the genetic material of the donor, in a detectable manner.

The methods described (e.g., 100, 100b, 100c) can, however, include other suitable steps and/or enable other downstream applications.

For instance, in another specific use case, the methods can be adapted for evaluation of minimal residual disease (MRD) based upon detection of numbers of cancer cell targets present in a sample from a subject after one or more phases of cancer treatment (e.g., treatment of leukemia, treatment of lymphoma, treatment of multiple myeloma, etc.).

In another specific use case, the methods can be adapted for single nucleotide polymorphism genotyping (SNPtyping) to measure genetic variations of SNPs between members (e.g., members of a species). Additionally, the method(s) can be used for single nucleotide variant genotyping (SNVtyping) for germline DNA samples.

The method(s) can also be used for applications involving disease prediction generation and monitoring with multiplexed detection of markers of a gene expression marker panel (e.g., for pregnancy-associated complications, for other applications).

In another specific use case, the methods can be adapted for ribosomal 16S and/or ITS characterization, where sequencing technologies may be fraught with high false positive rates and/or high PCR error. In relation to the specific use case, systems, methods, and compositions described can be used to disperse a sample of 16S and/or ITS ribosomal RNA (rRNA) across a plurality of partitions (as described in more detail below), where processing materials described enable detection of regions/sequences of interest (e.g., V3 region, V4 region, V5, region, other hypervariable regions, etc.), and subsequently, for operational taxonomic unit (OTU) or amplicon sequence variant (ASV) categorizations. For instance, detection of V3, V4, and/or V5 regions can be used for bacterial microbiome analyses, fungal microbiome analyses, other microbiome analyses, rare species detection, and/or other applications. Additionally or alternatively, such rRNA characterizations can be used for antimicrobial susceptibility testing (e.g., with a sample having one or more antibiotics being assessed, combined with bacteria and materials that can be used to indicate bacteria responses to the antibiotic(s)). Additionally or alternatively, such rRNA characterizations can be used for detection of a set of pathogens (e.g., up to 30 pathogens, up to 40 pathogens, up to 50 pathogens, up to 60 pathogens, up to 70 pathogens, etc.) and quantification (e.g., in relation to detection of presence or absence of various pathogens, in relation to characterization of infectious agents and potential prognoses). Additionally or alternatively, for microbial pathogen detection/quantification, any part of microbial genomics of a sample (e.g., non-rRNA targets) can be targeted, and subsequent detection can involve detection of sample composition (e.g., microbial composition, microbiome composition, etc.) without performance of next generation sequencing (NGS). In a related use case, detection/quantification of targets of a sample in a multiplexed manner can be used to differentiate between viral, fungal, and/or microbial infections (e.g., for a respiratory illness panel).

Embodiments of the methods described can be further adapted for other applications of use.

An aspect of the disclosure provides a method. The method may comprise providing a plurality of partitions. Each partition of the plurality of partitions may comprise a portion of a nucleic acid sample, processing materials, or a combination thereof. A distribution of nucleic acid material of the nucleic acid sample to partition have an average amount of nucleic acid material per partition. The plurality of partitions comprises an average volume. The method may comprise reacting processing materials of each partition of the plurality of partitions with a portion of the nucleic acid sample of each partition of the plurality of partitions. The method may comprise detection signals from at least a subset of the plurality of partitions. The method may comprise using the detected signals to identify a plurality of nucleic acid molecules of the nucleic acid sample. In some cases, the distribution of nucleic acid material of the nucleic acid sample to partition may have an average amount of at least 10 femtograms (fg) of nucleic acid material per partition. In some cases, the plurality of partitions may comprise at least 1,000 partitions. In some cases, the plurality of partitions may comprise an average volume of at most 10 picolitres (pL).

Each partition of the plurality of partitions may comprise a volume. The average volume may be determined by determining a mean, a median, an average, or a combination thereof based on the volumes of each partition within a plurality of partitions. The average volume of the plurality of partitions may be at least about 0.01 pL, at least about 0.05 pL, at least about 0.1 pL, at least about 0.5 pL, at least about 1 pL, at least about 5 pL, at least about 10 pL, at least about 15 pL, at least about 20 pL, at least about 25 pL, at least about 30 pL, at least about 35 pL, at least about 40 pL, at least about 45 pL, at least about 50 pL, at least about 55 pL, at least about 60 pL, at least about 65 pL, at least about 70 pL, at least about 75 pL, at least about 80 pL, at least about 85 pL, at least about 90 pL, at least about 95 pL, at least about 100 pL, at least about 125 pL, at least about 150 pL, at least about 200 pL, at least about 250 pL, at least about 300 pL, at least about 400 pL, at least about 500 pL, at least about 600 pL, at least about 700 pL, at least about 800 pL, at least about 900 pL, at least about 1000 pL, at least about 10000 pL, or more. The average volume of the plurality of partitions may be at most about 0.01 pL, at most about 0.05 pL, at most about 0.1 pL, at most about 0.5 pL, at most about 1 pL, at most about 5 pL, at most about 10 pL, at most about 15 pL, at most about 20 pL, at most about 25 pL, at most about 30 pL, at most about 35 pL, at most about 40 pL, at most about 45 pL, at most about 50 pL, at most about 55 pL, at most about 60 pL, at most about 65 pL, at most about 70 pL, at most about 75 pL, at most about 80 pL, at most about 85 pL, at most about 90 pL, at most about 95 pL, at most about 100 pL, at most about 125 pL, at most about 150 pL, at most about 200 pL, at most about 250 pL, at most about 300 pL, at most about 400 pL, at most about 500 pL, at most about 600 pL, at most about 700 pL, at most about 800 pL, at most about 900 pL, at most about 1000 pL, at most about 10000 pL, or less. The average volume of the plurality of partitions may be about 1-700 pL, about 5-600 pL, about 10⁻⁵⁰⁰ pL, about 15-400 pL, about 20-300 pL, about 25-250 pL, about 30-200 pL, about 35-150 pL, about 40-125 pL, about 45-100 pL, about 50-95 pL, about 55-90 pL, about 60-85 pL, about 65-80 pL, or about 70-75 pL.

Another aspect of the disclosure provides a method. The method may comprise providing a plurality of partitions comprising at least 1,000,000 partitions. Each partition of the plurality of partitions may comprise a portion of a nucleic acid sample, processing materials, or a combination thereof. A distribution of nucleic acid material of the nucleic acid sample to partition may have an average amount of nucleic acid material per partition. The method may comprise reacting processing materials of each partition of the plurality of partitions with a portion of the nucleic acid sample of each partition of the plurality of partitions. The method may comprise detection signals from at least a subset of the plurality of partitions. The method may comprise using the detected signals to identify a plurality of nucleic acid molecules of the nucleic acid sample. In some cases, the distribution of nucleic acid material of the nucleic acid sample to partition may have an average amount of at least 10 femtograms (fg) of nucleic acid material per partition.

The distribution of nucleic acid material to partition may have different average amounts of nucleic acid sample per partition. In some cases, the distribution of nucleic acid material to partition may have an average amount of nucleic acid sample per partition of at least about 0.01 fg, at least about 0.05 fg, at least about 0.1 fg, at least about 0.5 fg, at least about 1 fg, at least about 5 fg, at least about 10 fg, at least about 15 fg, at least about 20 fg, at least about 25 fg, at least about 30 fg, at least about 35 fg, at least about 40 fg, at least about 45 fg, at least about 50 fg, at least about 55 fg, at least about 60 fg, at least about 65 fg, at least about 70 fg, at least about 75 fg, at least about 80 fg, at least about 85 fg, at least about 90 fg, at least about 95 fg, at least about 100 fg, at least about 125 fg, at least about 150 fg, at least about 200 fg, at least about 250 fg, at least about 300 fg, at least about 400 fg, at least about 500 fg, at least about 600 fg, at least about 700 fg, at least about 800 fg, at least about 900 fg, at least about 1000 fg, at least about 10000 fg, or more. In some cases, the distribution of nucleic acid material to partition may have an average amount of nucleic acid sample per partition of at most about 0.01 fg, at most about 0.05 fg, at most about 0.1 fg, at most about 0.5 fg, at most about 1 fg, at most about 5 fg, at most about 10 fg, at most about 15 fg, at most about 20 fg, at most about 25 fg, at most about 30 fg, at most about 35 fg, at most about 40 fg, at most about 45 fg, at most about 50 fg, at most about 55 fg, at most about 60 fg, at most about 65 fg, at most about 70 fg, at most about 75 fg, at most about 80 fg, at most about 85 fg, at most about 90 fg, at most about 95 fg, at most about 100 fg, at most about 125 fg, at most about 150 fg, at most about 200 fg, at most about 250 fg, at most about 300 fg, at most about 400 fg, at most about 500 fg, at most about 600 fg, at most about 700 fg, at most about 800 fg, at most about 900 fg, at most about 1000 fg, at most about 10000 fg, or less. In some cases, the distribution of nucleic acid material to partition may have an average amount of nucleic acid sample per partition of about 1-700 fg, about 5-600 fg, about 10-500 fg, about 15-400 fg, about 20-300 fg, about 25-250 fg, about 30-200 fg, about 35-150 fg, about 40-125 fg, about 45-100 fg, about 50-95 fg, about 55-90 fg, about 60-85 fg, about 65-80 fg, or about 70-75 fg. In some cases, the distribution of nucleic acid material to partition has an average of at least 100 fg of said nucleic acid sample per partition. In some cases, the distribution of nucleic acid material to partition has an average of at least 200 fg of said nucleic acid sample per partition.

In some cases, the plurality of partitions may comprise different number of partitions. The plurality of partitions may comprise at least 100, at least about 1,000, at least about 5,000, at least about 10,000, at least about 50,000, at least about 100,000, at least about 500,000, at least about 1,000,000, at least about 2,000,000, at least about 3,000,000, at least about 4,000,000, at least about 5,000,000, at least about 6,000,000, at least about 7,000,000, at least about 8,000,000, at least about 9,000,000, at least about 10,000,000, at least about 11,000,000, at least about 12,000,000, at least about 13,000,000, at least about 14,000,000, at least about 15,000,000, at least about 16,000,000, at least about 17,000,000, at least about 18,000,000, at least about 19,000,000, at least about 20,000,000, at least about 21,000,000, at least about 22,000,000, at least about 23,000,000, at least about 24,000,000, at least about 25,000,000, at least about 26,000,000, at least about 27,000,000, at least about 28,000,000, at least about 29,000,000, at least about 30,000,000, at least about 31,000,000, at least about 32,000,000, at least about 33,000,000, at least about 34,000,000, at least about 35,000,000, at least about 36,000,000, at least about 37,000,000, at least about 38,000,000, at least about 39,000,000, at least about 40,000,000, at least about 41,000,000, at least about 42,000,000, at least about 43,000,000, at least about 44,000,000, at least about 45,000,000, at least about 46,000,000, at least about 47,000,000, at least about 48,000,000, at least about 49,000,000, at least about 50,000,000, at least about 60,000,000, at least about 70,000,000, at least about 80,000,000, at least about 90,000,000, at least about 100,000,000, or more partitions. The plurality of partitions may comprise at most 100, at most about 1,000, at most about 5,000, at most about 10,000, at most about 50,000, at most about 100,000, at most about 500,000, at most about 1,000,000, at most about 2,000,000, at most about 3,000,000, at most about 4,000,000, at most about 5,000,000, at most about 6,000,000, at most about 7,000,000, at most about 8,000,000, at most about 9,000,000, at most about 10,000,000, at most about 11,000,000, at most about 12,000,000, at most about 13,000,000, at most about 14,000,000, at most about 15,000,000, at most about 16,000,000, at most about 17,000,000, at most about 18,000,000, at most about 19,000,000, at most about 20,000,000, at most about 21,000,000, at most about 22,000,000, at most about 23,000,000, at most about 24,000,000, at most about 25,000,000, at most about 26,000,000, at most about 27,000,000, at most about 28,000,000, at most about 29,000,000, at most about 30,000,000, at most about 31,000,000, at most about 32,000,000, at most about 33,000,000, at most about 34,000,000, at most about 35,000,000, at most about 36,000,000, at most about 37,000,000, at most about 38,000,000, at most about 39,000,000, at most about 40,000,000, at most about 41,000,000, at most about 42,000,000, at most about 43,000,000, at most about 44,000,000, at most about 45,000,000, at most about 46,000,000, at most about 47,000,000, at most about 48,000,000, at most about 49,000,000, at most about 50,000,000, at most about 60,000,000, at most about 70,000,000, at most about 80,000,000, at most about 90,000,000, at most about 100,000,000, or fewer partitions. In some cases, the plurality of partitions may comprise about 100-100,000,000, about 1,000-90,000,000, about 5,000-80,000,000, about 10,000-70,000,000, about 50,000-60,000,000, about 100,000-50,000,000, about 500,000-49,000,000, about 1,000,000-48,000,000, about 2,000,000-47,000,000, about 3,000,000-46,000,000, about 4,000,000-45,000,000, about 5,000,000-44,000,000, about 6,000,000-43,000,000, about 7,000,000-42,000,000, about 8,000,000-41,000,000, about 9,000,000-40,000,000, about 10,000,000-39,000,000, about 11,000,000-38,000,000, about 12,000,000-37,000,000, about 13,000,000-36,000,000, about 14,000,000-35,000,000, about 15,000,000-34,000,000, about 16,000,000-33,000,000, about 17,000,000-32,000,000, about 18,000,000-31,000,000, about 19,000,000-30,000,000, about 20,000,000-29,000,000, about 21,000,000-28,000,000, about 22,000,000-27,000,000, about 23,000,000-26,000,000, or about 24,000,000-25,000,000. In some cases, the plurality of partitions may comprise at least about 10,000 partitions. In some cases, the plurality of partitions may comprise at least about 1,000,000 partitions.

The nucleic acid sample may comprise nucleic acid material. Nucleic acid material may comprise a variety of components including but not limited to nucleic acid molecules, nucleic acid fragments, chromosomes or fragments thereof, or a combination thereof. The nucleic acid material may comprise deoxyribonucleic acid (DNA), ribonucleic acid (RNA), xeno nucleic acid (XNA), locked nucleic acid (LNA), or a combination thereof. The RNA may comprise one or more types of RNA including but not limited to messenger RNA (mRNA), ribosomal RNA (rRNA), transfer RNA (tRNA), micro RNA (miRNA), or a combination thereof. The nucleic acid material may comprise single-stranded regions, double-stranded regions, or a combination thereof. The nucleic acid material may comprise one or more modifications. In some cases, the one of more modifications may be a label or moiety affixed to the nucleic acid material. For example, nucleic acid molecules of the nucleic acid material may comprise one or more dye modifications. In some cases, the one or more modifications may comprise modifications relative to a reference sequence. For example, a nucleic acid molecule of the nucleic acid material may comprise a mutation relative to a human reference sequence. In some cases, the reference sequence may comprise a reference genome, for example a human reference genome. In some cases, the reference sequence may comprise a healthy human genome. The one or more modifications may comprise one or more single nucleotide variant (SNVs), one or more single nucleotide polymorphisms (SNPs), one or more indels, one or more deletions, one or more mutations, or a combination thereof. In some cases, the one or more (SNVs) may comprise one of more C to G variants, one of more C to A variants, one of more C to T variants, A to G variants, one of more A to C variants, one of more A to T variants, G to A variants, one of more G to C variants, one of more G to T variants, T to A variants, one of more T to C variants, one of more T to G variants or a combination thereof. In some cases, the one or more modifications may comprise one or more mutations on one or more genes. For example, the one or more modifications may comprise one or more mutations on the KIT gene (e.g. a D816V mutation). In some cases, the one or more modifications may comprise one or more mutations on the JAK2 gene (e.g. a V617F mutation.)

The nucleic acid material may comprise one or more nucleic acid molecules. The one or more nucleic molecules of the nucleic acid sample may have a variety of sizes and lengths. Each of the one or more nucleic acid molecules of the nucleic acid sample in a sample may have the same size. In some cases, the one or more nucleic acid molecules of the nucleic acid sample may have different sizes. Each of the one or more nucleic acid molecules of the nucleic acid sample may have the same length. In some cases, the one or more nucleic acid molecules may have different lengths. The one or more nucleic acid molecules of the nucleic acid sample may have lengths of at least about 10 base pairs (bp), at least about 15 bp, at least about 20 bp, at least about 25 bp, at least about 30 bp, at least about 45 bp, at least about 50 bp, at least about 60 bp, at least about 70 bp, at least about 80 bp, at least about 90 bp, at least about 100 bp, at least about 110 bp, at least about 120 bp, at least about 130 bp, at least about 140 bp, at least about 150 bp, at least about 160 bp, at least about 170 bp, at least about 180 bp, at least about 190 bp, at least about 200 bp, at least about 210 bp, at least about 220 bp, at least about 230 bp, at least about 240 bp, at least about 250 bp, at least about 500 bp, at least about 1 (kilo base pairs) kb, at least about 2 kb, at least about 3 kb, at least about 4 kb, at least about 5 kb, at least about 10 kb, at least about 20 kb, at least about 50 kb, at least about 100 kb, or longer. The one or more nucleic acid molecules of the nucleic acid sample may have lengths of at most about 10 bp, at most about 15 bp, at most about 20 bp, at most about 25 bp, at most about 30 bp, at most about 45 bp, at most about 50 bp, at most about 60 bp, at most about 70 bp, at most about 80 bp, at most about 90 bp, at most about 100 bp, at most about 110 bp, at most about 120 bp, at most about 130 bp, at most about 140 bp, at most about 150 bp, at most about 160 bp, at most about 170 bp, at most about 180 bp, at most about 190 bp, at most about 200 bp, at most about 210 bp, at most about 220 bp, at most about 230 bp, at most about 240 bp, at most about 250 bp, at most about 500 bp, at most about 1 kb, at most about 2 kb, at most about 3 kb, at most about 4 kb, at most about 5 kb, at most about 10 kb, at most about 20 kb, at most about 50 kb, at most about 100 kb, or shorter. The one or more nucleic acid molecules of the nucleic acid sample may have lengths of at most about 10 bp-100 kb, about 15 bp-50 kb, about 20 bp-20 kb, about 25 bp-10 kb, about 30 bp-5 kb, about 45 bp-4 kb, about 50 bp-3 kb, about 60 bp-2 kb, about 70 bp-1 kb, about 80 bp-500 bp, about 90 bp-250 bp, about 100 bp-240 bp, about 110 bp-230 bp, about 120 bp-220 bp, about 130 bp-210 bp, about 140 bp-200 bp, about 150 bp-190 bp, or about 160 bp-180 bp.

The nucleic acid sample may comprise an amount of nucleic acids. For example, the nucleic acid sample may comprise 0.1 micrograms (ug) of nucleic acids. The amount of nucleic acids of the nucleic acid sample may be determined in a variety of ways. For example, an absorbance measurement of a nucleic acid sample may be acquired to measure a concentration of the nucleic acid material. A spectrophotometer may be used to measure an absorbance of the nucleic acid material. The absorbance at 260 nanometers (nm) may be used to determine the concentration of the nucleic acids within the nucleic acid sample. In some cases, a fluorescence dye may be used to determine the concentration, amount, or combination thereof of nucleic acids within the nucleic acid sample. For example, a DNA binding dye including Hoechst dyes, PicoGreen, QuantiFluor, or a combination thereof may be used to estimate the amount of nucleic acids within a nucleic acid sample. Gel electrophoresis may be used to determine an amount of nucleic acids within the nucleic acid sample. In some cases, more than one method may be used to determine the amount of nucleic acids in a nucleic acid sample.

The nucleic acid sample may comprise different amounts of nucleic acids. The nucleic acid sample may comprise at least about 0.001 μg, at least about 0.005 μg, at least about 0.01 μg, at least about 0.05 μg, at least about 0.1 μg, at least about 0.2 μg, at least about 0.3 μg, at least about 0.4 μg, at least about 0.5 μg, at least about 0.6 μg, at least about 0.7 μg, at least about 0.8 ug, at least about 0.9 μg, at least about 1 μg, at least about 1.1 μg, at least about 1.2 μg, at least about 1.3 μg, at least about 1.4 μg, at least about 1.5 μg, at least about 1.6 μg, at least about 1.7 ug, at least about 1.8 μg, at least about 1.9 μg, at least about 2 μg, at least about 2.5 μg, at least about 3 μg, at least about 3.5 μg, at least about 4 μg, at least about 5 μg, at least about 6 μg, at least about 7 μg, at least about 8 μg, at least about 9 μg, at least about 10 μg, at least about 15 μg, at least about 20 μg, at least about 25 μg, at least about 30 μg, at least about 50 μg, at least about 75 μg, at least about 100 μg, or more. The nucleic acid sample may comprise at most about 0.001 μg, at most about 0.005 μg, at most about 0.01 μg, at most about 0.05 μg, at most about 0.1 μg, at most about 0.2 μg, at most about 0.3 μg, at most about 0.4 μg, at most about 0.5 ug, at most about 0.6 μg, at most about 0.7 μg, at most about 0.8 μg, at most about 0.9 μg, at most about 1 μg, at most about 1.1 μg, at most about 1.2 μg, at most about 1.3 μg, at most about 1.4 μg, at most about 1.5 μg, at most about 1.6 μg, at most about 1.7 μg, at most about 1.8 μg, at most about 1.9 μg, at most about 2 μg, at most about 2.5 μg, at most about 3 μg, at most about 3.5 μg, at most about 4 μg, at most about 5 μg, at most about 6 μg, at most about 7 μg, at most about 8 μg, at most about 9 μg, at most about 10 μg, at most about 15 μg, at most about 20 μg, at most about 25 μg, at most about 30 μg, at most about 50 μg, at most about 75 μg, at most about 100 μg, or more. The nucleic acid sample may comprise about 0.001-100 μg, about 0.005-75 μg, about 0.01-50 μg, about 0.05-30 μg, about 0.1-25 μg, about 0.2-20 μg, about 0.3-15 μg, about 0.4-10 μg, about 0.5-9 μg, about 0.6-8 μg, about 0.7-7 μg, about 0.8-6 μg, about 0.9-5 μg, about 1-4 μg, about 1.1-3.5 μg, about 1.2-3 μg, about 1.3-2.5 μg, about 1.4-2 μg, about 1.5-1.9 μg, or about 1.6-1.8 μg. The nucleic acid sample comprises at least about 0.1 microgram of nucleic acid material. The nucleic acid sample comprises at least about 0.5 microgram of nucleic acid material. The nucleic acid sample comprises at least about 1.0 microgram of nucleic acid material.

The nucleic acid sample may comprise a variety of nucleic acid molecules. In some cases, the nucleic acid sample may comprise at least about 1×10³ nucleic acid molecules, at least about 1×10⁴ nucleic acid molecules, at least about 1×10⁵ nucleic acid molecules, at least about 1×10⁶ nucleic acid molecules, at least about 1×10⁷ nucleic acid molecules, at least about 1×10⁸ nucleic acid molecules, at least about 1×10⁹ nucleic acid molecules, at least about 1×10¹⁰ nucleic acid molecules, at least about 1×10¹¹ nucleic acid molecules, at least about 1×10¹² nucleic acid molecules, at least about 1×10¹³ nucleic acid molecules, at least about 1×10¹⁴ nucleic acid molecules, or more nucleic acid molecules. In some cases, the nucleic acid sample may comprise at most about 1×10³ nucleic acid molecules, at most about 1×10⁴ nucleic acid molecules, at most about 1×10⁵ nucleic acid molecules, at most about 1×10⁶ nucleic acid molecules, at most about 1×10⁷ nucleic acid molecules, at most about 1×10⁸ nucleic acid molecules, at most about 1×10⁹ nucleic acid molecules, at most about 1×10¹⁰ nucleic acid molecules, at most about 1×10¹¹ nucleic acid molecules, at most about 1×10¹² nucleic acid molecules, at most about 1×10¹³ nucleic acid molecules, at most about 1×10¹⁴ nucleic acid molecules, or more nucleic acid molecules.

The nucleic acid sample may comprise cell-free nucleic acids (cfNA). The cfNA may comprise cell-free RNA, cell-free DNA (cfDNA), or a combination thereof. The nucleic acid sample comprising cfNA may be prepared from a bodily fluid. The bodily fluid may comprise blood, serum, saliva, urine, cerebrospinal fluid, bone marrow, or a combination thereof. For example, a blood sample may be processed into separate layers using centrifugation. The separate layers may include a plasm layer, a buffy coat layer, a red blood cell layer, or a combination thereof. The cfNA may be extracted from the plasma layer.

The plurality of partitions may be generated in a variety of ways. In some cases, the nucleic acid sample may be loaded into a spin column. The spin column containing the nucleic acid sample may be centrifuged into a collection container containing one or more fluids to generate the plurality of partitions. The spin column may comprise a membrane with pores. In some cases, the spin column may comprise a fluid prior to loading the nucleic acid sample into the spin column. The fluid may be immiscible with the nucleic acid sample such that upon addition of the nucleic acid sample, there is more than one fluid layer. In some cases, the nucleic acid sample may be loaded into the spin column using a pipette.

The collection container may have any suitable shape, dimensions, size, composition, etc. The collection container may comprise a tube, a cuvette, a receptacle, a bucket, a reservoir, or a vial. For example, in some cases, the collection container may comprise a polymerase chain reaction (PCR) tube. The collection container may comprise a microcentrifuge tube. The collection container may comprise one or more fluid layers. In some cases, the collection container may comprise an aqueous fluid layer. The aqueous fluid layer may comprise an index of refraction that is similar or equivalent to the index of refraction of the nucleic acid sample.

The collection container may comprise a fluid layer that is immiscible with the aqueous fluid layer of the collection container. The aqueous fluid layer may be below the fluid layer that is immiscible with the aqueous fluid layer of the collection container. The aqueous fluid layer may be above the fluid layer that is immiscible with the aqueous fluid layer of the collection container.

A centrifuge may be used to spin the spin column through the membrane into the collection tube. In some cases a swinging bucket centrifuge may be used. The spin column and the collection container may be placed within a holder during the centrifuging. In some cases. The nucleic acid sample within the spin column may be rotated in a first direction and then rotated in a second direction. The plurality of partitions may be generated by driving a solution comprising the nucleic acid sample through the membrane. The plurality of partitions may comprise a plurality of droplets.

The plurality of partitions may be present in the collection container. The plurality of partitions may be part of a gel, a gel matrix, an emulsion, or a combination thereof. In some cases, the emulsion may comprise a double emulsion. For example, the partitions of the plurality of partitions may comprise an aqueous solution. The aqueous solution of each of the partitions may be surrounded by an immiscible film. The plurality of partitions may be present in a continuous phase. The continuous phase may be miscible with the aqueous solutions of each of the partitions of the plurality of partitions and may be immiscible with the immiscible film of each of the partitions of the plurality of partitions.

The plurality of partitions may be immobilized in the collection container. For example, the plurality of partitions may be part of a gel matrix that is immobilized in the collection container. The gel matrix may comprise a consistency such that a partition of the plurality of partitions does not move substantially in response to a perturbation (e.g. a movement of the collection container). The plurality of partitions may remain in essentially the same position (e.g. the same x, y, and z, positions) within the collection container in response to a movement and/or jostling of the collection container. For example, the collection container may be lifted, shifted in location, rotated, or a combination thereof and the plurality of partitions may not move relative to the original location of the partitions of the plurality of partitions as a result of the movement and/or jostling of the collection container. In some cases, the plurality of partitions may be immobilized in the collection container during different parts of the method. For example, the plurality of partitions may be immobilized in the collection container after generation of the plurality of partitions, before performing reactions, during reacting, after performing reactions, before detecting signals, during detecting signals, after detecting signals, or a combination thereof. In some cases, the plurality of partitions may be immobilized in the container during reacting the processing materials of each partition of the plurality of partitions with the portion of the nucleic acid sample of each partition of the plurality of partitions. In some cases, the plurality of partitions may be immobilized in the container while detecting signals from at least a subset of partitions of the plurality of partitions.

The methods described herein may further comprise incubating the plurality of partitions. In some cases, the plurality of partitions may be incubated after generation of the plurality of partitions, before performing reactions, during reacting, after performing reactions, before detecting signals, during detecting signals, after detecting signals, or a combination thereof. The plurality of partitions may be incubated for at least about 10 minutes, at least about 20 minutes, at least about 30 minutes, at least about 45 minutes, at least about 1 hour, at least about 2 hours, at least about 3 hours, at least about 5 hours, at least about 10 hours, at least about 20 hours, at least about 1 day, at least about 2 days, at least about 3 days, at least about 5 days, at least about 1 week, at least about 1 month, or longer. The plurality of partitions may be incubated for at most about 10 minutes, at most about 20 minutes, at most about 30 minutes, at most about 45 minutes, at most about 1 hour, at most about 2 hours, at most about 3 hours, at most about 5 hours, at most about 10 hours, at most about 20 hours, at most about 1 day, at most about 2 days, at most about 3 days, at most about 5 days, at most about 1 week, at most about 1 month, or shorter. The plurality of partitions may be incubated for about 10 minutes-1 month, about 20 minutes-1 week, about 30 minutes-5 days, about 45 minutes-3 days, about 1 hour-2 days, about 2 hours-1 day, about 3 hours-20 hours, or about 5 hours-10 hours.

The plurality of partitions may be incubated at a temperature. The temperature may be at least about −80° C., at least about −20° C., at least about −5° C., at least about 0° C., at least about 2° C., at least about 4° C., at least about 6° C., at least about 8° C., at least about 10° C., at least about 12° C., at least about 14° C., at least about 16° C., at least about 18° C., at least about 20° C., at least about 22° C., at least about 24° C., at least about 26° C., at least about 28° C., at least about 30° C., at least about 32° C., at least about 34° C., at least about 36° C., at least about 38° C., at least about 40° C., at least about 45° C., at least about 50° C., at least about 55° C., at least about 60° C., at least about 65° C., at least about 70° C., at least about 75° C., at least about 80° C., at least about 85° C., or at least about 90° C., or higher. The temperature may be at most about −80° C., at most about −20° C., at most about −5° C., at most about 0° C., at most about 2° C., at most about 4° C., at most about 6° C., at most about 8° C., at most about 10° C., at most about 12° C., at most about 14° C., at most about 16° C., at most about 18° C., at most about 20° C., at most about 22° C., at most about 24° C., at most about 26° C., at most about 28° C., at most about 30° C., at most about 32° C., at most about 34° C., at most about 36° C., at most about 38° C., at most about 40° C., at most about 45° C., at most about 50° C., at most about 55° C., at most about 60° C., at most about 65° C., at most about 70° C., at most about 75° C., at most about 80° C., at most about 85° C., or at most about 90° C., or less. The temperature may be about −80° C. to 90° C., about −20° C. to 85° C., about −5° C. to 80° C., about 0° C. to 75° C., about 2° C. to 70° C., about 4° C. to 65° C., about 6° C. to 60° C., about 8° C. to 55° C., about 10° C. to 50° C., about 12° C. to 45° C., about 14° C. to 40° C., about 16° C. to 38° C., about 18° C. to 36° C., about 20° C. to 34° C., about 22° C. to 32° C., about 24° C. to 30° C., or about 26° C. to 28° C. In some cases, the plurality of partitions may be incubated prior to performing a reaction at a temperature less than or equal to 20° C. for at least 1 hour. In some cases, the plurality of partitions may be incubated prior to detecting signals at a temperature less than or equal to 20° C. for at least 1 hour. In some cases, the plurality of partitions may be incubated prior to using detected signal to identify the plurality of nucleic acid molecules at a temperature less than or equal to 20° C. for at least 1 hour.

The sample used to generate the plurality of partitions may comprise the nucleic acid sample, processing materials, or a combination thereof. The partitions of the plurality of partitions may comprise the processing materials. The partitions of the plurality of partitions may comprise a portion of the nucleic acid sample, processing materials, or a combination thereof. The processing materials may comprise a variety of components. The processing materials may comprise one or more enzymes. The one or more enzymes of the processing materials may be used to interact with (e.g. react with) the nucleic acid sample or portion thereof. For example, the enzyme may be used to amplify the portion of the nucleic acid material of each partition of the plurality of partitions. In some cases, the one or more enzymes may comprise one or more polymerases, one or more helicases, one or more nickases, one or more ligases, or a combination thereof. The one or more polymerases of the one or more enzymes of the processing materials may comprise a DNA polymerase, an RNA polymerase, or a combination thereof. The DNA polymerase may comprise Q5 High-Fidelity DNA Polymerase, Q5U Hot Start High-Fidelity DNA Polymerase, Bsu DNA Polymerase, Large Fragment, phi29 DNA Polymerase, phi29-XT DNA Polymerase, T7 DNA Polymerase (unmodified), Sulfolobus DNA Polymerase IV, Therminator™ DNA Polymerase, DNA Polymerase I (E. coli), DNA Polymerase I, Large (Klenow) Fragment′, Klenow Fragment (3′→5′ exo−), T4 DNA Polymerase, Legacy Polymerases, Vent DNA Polymerase, Vent (exo−) DNA Polymerase, Deep Vent DNA Polymerase, Deep Vent (exo-) DNA Polymerase, Phusion High-Fidelity DNA Polymerase*, Routine PCR, OneTaq DNA Polymerase, Taq DNA Polymerase, LongAmp Taq DNA Polymerase, Hemo KlenTaq, Epimark Hot Start Taq DNA Polymerase, Isothermal Amplification and Strand Displacement, Bst DNA Polymerase, Bst DNA Polymerase, Bst 2.0 DNA Polymerase, Bst 3.0 DNA Polymerase, SuperFi II DNA Polymerase, Platinum SuperFi II, Dream Taq polymerase, or a combination thereof.

The processing materials may comprise one or more buffers. The one or more buffers may comprise one or more buffering reagents, one or more salts, water, detergents, or a combination thereof. In some cases, the one or more buffering reagents of the one or more buffers of the processing materials may comprise 2-(N-morpholino) ethanesulfonic acid, (MES), bis-tris methane, (Bis-Tris), N-(2-acetamido)iminodiacetic acid, (ADA), N-(2-acetamido)-2-aminoethanesulfonic acid, (ACES), 1,4-Piperazinediethanesulfonic acid, (PIPES), 3-morpholinopropanesulfonic acid, (MOPSO), Bis-Tris Propane, (Bis-Tris Propane), N,N-Bis(2-hydroxyethyl)-2-aminoethanesulfonic acid, (BES), 3-(N-Morpholino) propanesulfonic acid, (MOPS), N-[Tris(hydroxymethyl)methyl]-2-aminoethanesulfonic acid, (TES), 2-[4-(2-hydroxyethyl) piperazin-1-yl]ethanesulfonic acid, (HEPES), 4-(N-Morpholino) butanesulfonic acid, (MOBS), 3-[N-tris(hydroxymethyl)methylamino]-2-hydroxypropanesulfonic acid, (TAPSO), Tris(hydroxymethyl)aminomethane, (Tris), N-(2-Hydroxyethyl) piperazine-N′-(2-hydroxypropanesulphonic acid), (HEPPSO), piperazine-1,4-bis(2-hydroxypropanesulfonic acid), (POPSO), Tris-acetate-EDTA, (TEA), N-tris(hydroxymethyl)methylglycine, (Tricine), (2-Aminoacetamido) acetic acid, (Gly-Gly), N,N-bis(2-hydroxyethyl)glycine, (Bicine), ([tris(hydroxymethyl)methylamino]propanesulfonic acid), (TAPS), 2-amino-2-methyl-1,3-propanediol, (AMPD), Phosphate buffered saline, (PBS), or a combination thereof.

The processing materials may comprise one or more salts. The one or more salts of the processing materials may comprise sodium chloride (NaCl), calcium chloride (CaCl₂), magnesium chloride (MgCl₂), potassium chloride (KCl), Ethylenediaminetetraacetic acid (EDTA), or a combination thereof. The processing materials may comprise one or more detergents. The one or more detergents may comprise polysorbate 20 (Tween 20), Octoxynol-9; toctylphenoxypolyethoxyethanol (Triton X-100™), octylphenoxypolyethoxyethanol (NP-40), or a combination thereof. The processing materials may a blocking component, including bovine serum albumin (BSA), sheared salmon sperm DNA, glycine, dimethyl sulfoxide (DMSO), or a combination thereof.

The processing materials may comprise one or more probes. The one or more probes of the processing materials may comprise nucleic acid. For example, the one or more probes of the processing materials may comprise one or more oligonucleotides. The one or more oligonucleotides may be complementary to at least a portion of the nucleic acid material of the nucleic acid samples. In some cases the one or more probes may comprise primers for use in a polymerase chain reaction (PCR). For example, the one or more primers of the one or more probes of the processing materials may comprise a sequence that is complementary to at least a portion of a nucleic acid molecule of the plurality of nucleic acid molecules of the nucleic acid sample. The one or more primers of the one or more probes of the processing materials may comprise a sequence that is not complementary to at least a portion of a nucleic acid molecule of the plurality of nucleic acid molecules of the nucleic acid sample. In some cases, the sequence that is not complementary to at least a portion of the nucleic acid molecule of the plurality of nucleic acid molecules of the nucleic acid sample may comprise a sequence that creates a single-strand overhang when a primer of the one or more primers of the one or more probes of the processing materials hybridizes to the nucleic acid molecule of the plurality of nucleic acid molecules of the nucleic acid sample. The single-strand overhang of the primer of the one or more primers may be at a 5′ end of the primer of the one or more primers, 3′ end of the primer of the one or more primers, or a combination thereof.

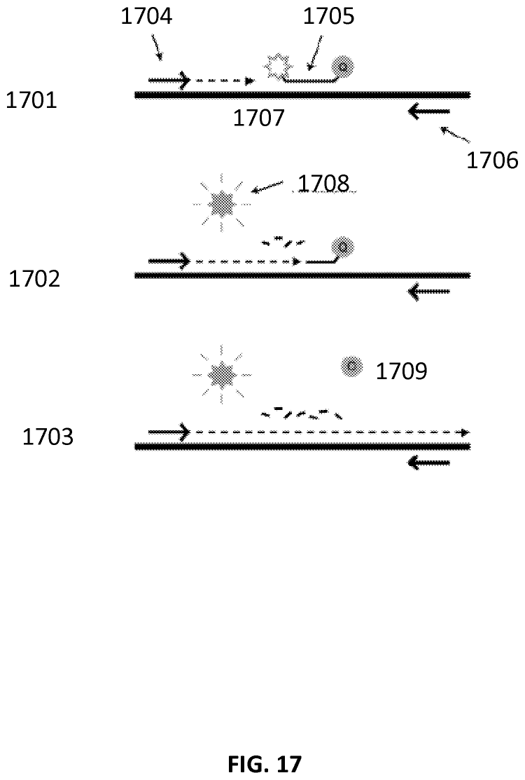

In some cases, the one or more primers may comprise a pair of primers for a loci. In some cases, the one or more probes may comprise a hydrolysis probe. In some cases, the one or more probes may comprise one or more pairs of primers for one or more loci and one or more hydrolysis probes. For example, a pair of primers and a hydrolysis probe may be included in the processing materials and may be used to detect a signal associated with a loci (FIG. 17). The pair of primers may comprise a forward primer (1704) and a reverse primer (1706). The forward and reverse primers my bind to the loci. The hydrolysis probe (1705) may bind to the loci in between where the forward and reverse primers bind to the loci. The hydrolysis probe may comprise a fluorescent modification and a quencher modification. An amplification reaction may be performed using the forward and reverse primers. The sequence of the forward primer may be extended using a polymerase (1701). The hydrolysis probe may be bound to the loci. The hydrolysis probe may be degraded during the extension process when the polymerase is in proximity to the hydrolysis probe (1702). The degradation of the hydrolysis probe may result in release of the fluorescent modification from the hydrolysis probe to generate an unbound fluorescent modification (1708). The hydrolysis probe may be fully degraded during the amplification reaction (1703). The full degradation of the hydrolysis probe may result in the generation of an unbound quencher modification (1709). The amplification reaction may result in detectable signal because of the generation of the unbound fluorescent modification (1708.)

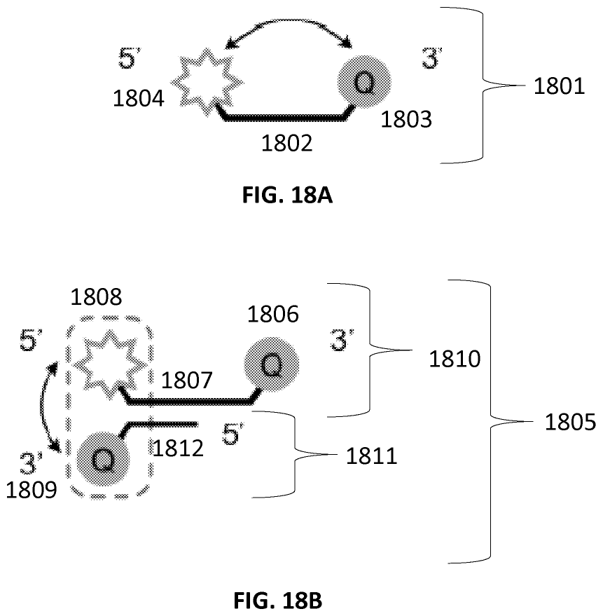

The one or more probes of the processing materials may comprise one or more hydrolysis probes (1801) (FIG. 18A). In some cases, the one or more hydrolysis probes may comprise one or more fluorescent modifications (1804), one or more quencher modifications (1803), one or more sequences that may bind to a loci (1802), or a combination thereof. In some cases, the one or more hydrolysis probes (1810) may be bound by one or more quencher probes (1809) to form a complex (1805) (FIG. 18B). The one or more hydrolysis probes may comprise one or more fluorescent modifications (1808), one or more quencher modifications (1806), one or more sequences that may bind to a loci (1807), or a combination thereof. The one or more quencher probes may comprise one or more quencher modifications (1809), one or more sequences that binds to one or more hydrolysis probes (1812), or a combination thereof. The one or more quencher probes may serve to quench the signal associated with the one or more fluorescent modifications of the one or more hydrolysis probes when the one or more quencher probes is bound to the one or more hydrolysis probes. The one or more quencher modifications of the one of more hydrolysis probes may serve to quench the signal associated with the one or more fluorescent modifications of the one or more hydrolysis probes.

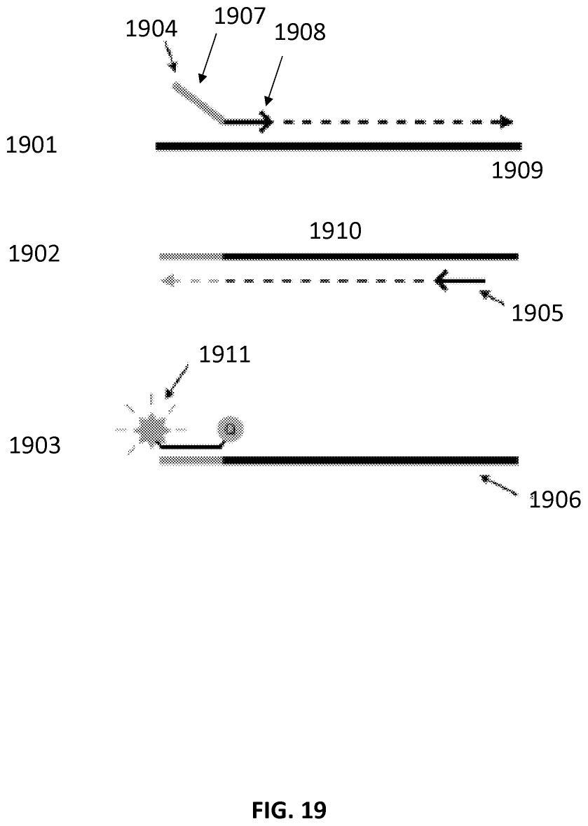

In some cases, the one or more probes of the processing materials may comprise a pair of primers and a hydrolysis probe (FIG. 19). The pair of primers may comprise a forward primer (1904) and a reverse primer (1905). The forward primer may comprise a sequence that binds to a loci (1909) and a sequence that does not bind to the loci (1907). An amplification reaction (1901) may be performed to extend the sequence that binds to the target of the forward primer based on the sequence of the loci to generate a sequence that includes the sequence that does not bind the loci (1907) and the reverse complement of at least a portion of the loci (1910). The reverse primer (1905) may bind to sequence 1910 and an amplification reaction (1902) may be performed to generate a reverse complement sequence (1906). The hydrolysis probe (1911) may bind to the sequence of 1906 corresponding to the sequence of the forward primer that does not bind to the loci. The hydrolysis probe may comprise a fluorescent modification, a quencher modification, a sequence that binds the sequence corresponding to the sequence of the forward primer that does not bind to the loci, or a combination thereof.

In some cases, the processing materials may comprise one or more primers for each loci identified in the methods described herein. For example, five loci may be identified using the methods described herein by identifying a plurality of nucleic acid molecules. In some cases the nucleic acid molecules of the plurality of nucleic acid molecules may comprise at least a portion of the sequence associated at least one of the five loci. The five loci may comprise sequence of interest, for example a gene sequence associate with a mutation. In some cases, the processing materials may comprise a pair of primers for each different loci. The pair of rimers may comprise a first primer and a second primer. The first primer of the pair of primers may comprise a sequence that is complementary to a 5′ end of a loci. The second primer of the pair of primers may comprise a sequence that is complementary to a 3′ end of the same loci. In some cases, the 5′ end of the loci and the 3′ end of the loci may be separated by a portion of the loci. In some cases, the portion of the loci may comprise at least about 4 bp, at least about 6 bp, at least about 8 bp, at least about 10 bp, at least about 12 bp, at least about 14 bp, at least about 15 bp, at least about 20 bp, at least about 25 bp, at least about 30 bp, at least about 35 bp, at least about 40 bp, at least about 45 bp, at least about 50 bp, at least about 60 bp, at least about 70 bp, at least about 80 bp, at least about 90 bp, at least about 100 bp, at least about 110 bp, at least about 120 bp, at least about 130 bp, at least about 140 bp, at least about 150 bp, at least about 160 bp, at least about 170 bp, at least about 180 bp, at least about 190 bp, at least about 200 bp, at least about 250 bp, at least about 300 bp, at least about 350 bp, at least about 400 bp, at least about 450 bp, at least about 500 bp, or more. In some cases, the portion of the loci may comprise at most about 4 bp, at most about 6 bp, at most about 8 bp, at most about 10 bp, at most about 12 bp, at most about 14 bp, at most about 15 bp, at most about 20 bp, at most about 25 bp, at most about 30 bp, at most about 35 bp, at most about 40 bp, at most about 45 bp, at most about 50 bp, at most about 60 bp, at most about 70 bp, at most about 80 bp, at most about 90 bp, at most about 100 bp, at most about 110 bp, at most about 120 bp, at most about 130 bp, at most about 140 bp, at most about 150 bp, at most about 160 bp, at most about 170 bp, at most about 180 bp, at most about 190 bp, at most about 200 bp, at most about 250 bp, at most about 300 bp, at most about 350 bp, at most about 400 bp, at most about 450 bp, at most about 500 bp, or less. In some cases, the portion of the loci may comprise about 4-500 bp, about 6-450 bp, about 8-400 bp, about 10-350 bp, about 12-300 bp, about 14-250 bp, about 15-200 bp, about 20-190 bp, about 25-180 bp, about 30-170 bp, about 35-160 bp, about 40-150 bp, about 45-140 bp, about 50-130 bp, about 60-120 bp, about 70-110 bp, or about 80-100 bp.

The one or more probes of the processing materials may comprise one or more probes comprising one or more modifications. The one or more modifications may comprise one or more fluorescent modifications, one or more quencher modifications, or a combination thereof. For example, the processing materials may comprise one or more probes comprising a fluorescent modification. In some cases, one or more probes of the processing materials may comprise both a fluorescent modification and a quencher modification. For example, a probe of the one or more probes of the processing materials may comprise a fluorescent modification and a quencher modification. The fluorescent modification may be on one end of the probe of the one or more probes of the processing materials (e.g. a 5′-end) and the quencher modification may be on another end of the probe of the one or more probes of the processing materials (e.g. a 3′ end). In some cases, the probe of the one or more probes of the processing materials comprising the fluorescent modification and the quencher modification may complement at least a portion of one or more loci detected using the methods described herein. For example, the probe of the one or more probes of the processing materials comprising the fluorescent modification and the quencher modification may comprise a sequence that hybridizes to a sequence of the loci. In some cases, the one or more probes of the processing materials may comprise a probe comprising a fluorescent modification and a quencher modification for each loci detected using the methods described herein. The probes comprising a fluorescent modification and a quencher modification for each loci detected using the methods described herein may comprise the same fluorescent modification or different fluorescent modifications. For example, each probe comprising a fluorescent modification and a quencher modification for each loci detected using the methods described herein may comprise a different fluorescent modification (e.g. each probe comprises a different type of fluorescent modification). In some cases, the one or more probes of the processing materials may comprise a probe comprising a quencher modification. The probe comprising the quencher modification of the one of more probes of the processing materials may be complementary to another probe of the one or more probes of the processing materials. For example, the probe comprising the quencher modification of the one of more probes of the processing materials may be complementary to a probe comprising a fluorescent modification and a quencher modification. In some cases, the one or more probes of the processing materials may bind to one or more nucleic acid molecules of the plurality of nucleic acid molecules of the nucleic acid sample.

The one or more fluorescent modifications of the one or more probes of the processing materials may comprise AlexaFluor Texas Red, rhodamine B, rhodamine 6G, rhodamine 10, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE, TMR, lissamine rhodamine, TEX 615, TYE™ 665, TYE 705, SUN, ATTO™ 425, ATTO™ 488, ATTO™ 532, ATTO™ 550, ATTO™ 565, ATTO™ Rho101, ATTO™ 590, ATTO™ 633, ATTO™ 647, ATTO™ 700, Alexa Fluor® 488 (NHS Ester), Alexa Fluor® 532 (NHS Ester), Alexa Fluor® 546 (NHS Ester), Alexa Fluor® 594 (NHS Ester), Alexa Fluor® 647 (NHS Ester), Alexa Fluor® 660 (NHS Ester), Alexa Fluor® 750 (NHS Ester), IRDye® 700, IRDye® 800, Rhodamine Red™, 5-TAMRA™, Texas Red®-X, Lightcycler® 640, Dy 750, Cascade Blue and derivatives such as Cascade Blue acetyl azide, Cascade Blue cadaverine, Cascade Blue ethylenediamine, Cascade Blue hydrazide, Lucifer Yellow, Lucifer Yellow CH, cyanine and derivatives such as indolium based cyanine dyes, benzo-indolium based cyanine dyes, Oregon Green dyes, WellRED dyes, IRD dyes, phycoerythrin and phycobilin dyes, Malachite green, stilbene, DEG dyes, Cy3, Cy5, and Cy7, fluorescein, or a combination thereof. The one or more quencher modifications of the one or more probes of the processing materials may comprise black hole quencher 2, black hole quencher 3, iowa black RQ, ZEN, or a combination thereof.

The processing materials may comprise one or more nucleotides. The one of more nucleotides of the processing materials may be used in PCR reactions. The one or more nucleotides of the processing materials may comprise natural nucleotides, non-natural nucleotides, or a combination thereof. The one or more nucleotides may comprise adenine (A), cytoside (C), guanine (G), thymine (T), uracil (U), 2-amino-6-(N,N-dimethylamino) purine, 2-amino-(6-thienyl) purine, N1-Methyl-Pseudouridine-5′-Triphosphate, 5-Methoxyuridine-5′-Triphosphate, Pseudouridine-5′-Triphosphate, 5-Methylcytidine-5′-Triphosphate, deoxycytidine triphosphate, deoxyguanosine triphosphate, thymidine triphosphate, deoxyadenosine triphosphate, or a combination thereof.

Reacting processing materials of the plurality of partitions with a portion of the nucleic acid sample of each partition of the plurality of partitions may comprise performing an amplification reaction. In some cases, the amplification reaction may comprise a PCR reaction, a quantitative PCR (qPCR) reaction, or a combination thereof. The amplification reaction may comprise heating the plurality of partitions. The plurality of partitions may be in a container during the amplification reaction. In an example, the container comprises a tube. In some cases, the amplification reaction of the plurality of partitions may comprise cooling the plurality of partitions. For example, the plurality of partitions may be cooled to a temperature below a temperature used to heat the plurality of partitions. In some cases, the plurality of partitions may be heated and cooled more than once during the amplification reaction. The amplification reaction may take place in a thermocycler instruction. The thermocycler instrument may automatically control the temperature and incubation time at each temperature of the plurality of droplets. The amplification reaction may result in the generating of multiple copies of nucleic acid molecules of the nucleic acid materials. The multiple copies of the nucleic acid molecules of the nucleic acid materials may comprise copies of the loci being detected in the methods describe herein. The amplification reaction may result in fluorescence signal. In some cases, the amplification reaction may comprise amplification of at least a portion of the nucleic acid materials of one or more partitions of the plurality of partitions. The amplification of the at least a portion of the nucleic acid materials of one or more partitions of the plurality of partitions may cause a fluorescent modification of one or more probes of the processing materials to be cleaved from the one or more probes. The cleavage of the fluorescent modification of the one or more probes may result in the fluorescent modification being physically separate from a quencher modification. The quencher modification may quench the fluorescence signal if in close proximity. The quencher modification may not quench the fluorescence signal if not in close proximity of the fluorescence modification. In some cases, the amplification reaction may cause a fluorescence modification of one or more probes of the processing material to not be in close proximity to a quencher modification resulting in signal. The signal of the fluorescence modification not in proximity to the quencher modification may be the result of a loci being present within a partition of the plurality of partitions.

The plurality of partitions may be imaged or otherwise optically interrogated. In some cases, the plurality of partitions may be imaged after performing an amplification reaction. In some cases, a subset of the plurality of partitions may be imaged. In some cases, all of the plurality of partitions may be imaged. The plurality of partitions may be immobilized within a collection container during imaging. In some cases, the plurality of partitions may be immobilized within the same container during the amplification reaction. The imaging may comprise scanning cross-sections of the plurality of containers immobilized within the container. The imaging may comprise using an imaging system. The imaging system may comprise light sheet imaging. In some cases, the imaging system may comprise a microscope, a stage, a fluidics module, a computer configured to execute imaging protocols, or a combination thereof. In some cases, the imaging system may comprise a light sheet microscope, a fluorescence microscope, a brightfield microscope, a confocal microscope, or a combination thereof. Examples of light sheet imaging systems are described in PCT/US2020/064117 and PCT/US2021/027353 which are incorporated herein by reference.

The plurality of partitions may be imaged by collecting image data across a set of channels. The set of channels may comprise fluorescence channels. For example, image data may be collected across a set of wavelengths corresponding to absorbance wavelengths of fluorescence modifications included in the one or more probes of the processing materials. In some cases, image data may be collected across at least about 1, at least about 2, at least about 3, at least about 4, at least about 5, at least about 6, at least about 7, at least about 8, at least about 9, at least about 10, or more channels. In some cases, image data may be collected across at most about 1, at most about 2, at most about 3, at most about 4, at most about 5, at most about 6, at most about 7, at most about 8, at most about 9, at most about 10, or fewer channels.

The plurality of partitions may comprise detectable signals after performing an amplification reaction. The detectable signals (e.g. signals) may comprise fluorescence intensities associated with the plurality of nucleic acid molecules of the nucleic acid sample. For example, the amplification reaction may comprise performing qPCR. The qPCR may result in the formation of unbound fluorescence molecules (e.g. unbound fluorescence modifications from one or more probes of the processing materials). The unbound fluorescence molecules may indicate the presence of a nucleic acid molecule within a partition of the plurality of partitions. In some cases one or more fluorescence signals may be associated with a nucleic acid molecule of the plurality of nucleic acid molecules of the nucleic acid sample. For example, two different probes, each comprising a different fluorescent modification may be bound to a nucleic acid molecule. After performing the amplification reaction, the two different fluorescent modifications may be unbound in solution. The unbound different fluorescent modifications may each comprise a different detectable signal. The combination of the different detectable signals of the unbound different fluorescent modifications of the two different probes may provide information related to the identity of the nucleic acid molecule bound by the two different probes within the partition.

Detecting signals from the plurality of partitions can be used to identify the plurality of nucleic acid molecules of the nucleic acid sample. In some cases, a detectable signal (e.g. signal) may correspond to a nucleic acid molecule of the plurality of nucleic acid molecules of the nucleic acid sample. For example, a signal may be detected using an imaging system in a partition of the plurality of partitions. The signal may correspond to a loci based on the sequence of the loci and sequence of probes of the one or more probes of the processing material included in the partition of the plurality of partitions. In some cases, one or more detectable signals (e.g. signals) may correspond to a nucleic acid molecule of the plurality of nucleic acid molecules of the nucleic acid sample. For example, two different signals may be detected using an imaging system in a partition of the plurality of partitions. The two different signals may correspond to a loci based on the sequences of the loci and sequences of probes of the one or more probes of the processing material included in the partition of the plurality of partitions. In some cases, identifying the plurality of nucleic acid molecules of the nucleic acid sample may comprise comparing said signals to a lookup table to identify said plurality of nucleic acid molecules of said nucleic acid sample. For example, signals detected in a partition of the plurality of partitions may be compared to a lookup table comprising combinations of signals that correspond to loci. The nucleic acid molecule may be identified as corresponding to a particular loci based on confirming the presence, absence, or combination thereof of signals included in the lookup table corresponding to the particular loci. The lookup table may comprise signal combinations include a total of at least about 1, at least about 2, at least about 3, at least about 4, at least about 5, at least about 6, at least about 7, at least about 8, at least about 9, at least about 10, at least about 15, at least about 20, at least about 25, or more different detectable signals (e.g. fluorescence channels). The lookup table may comprise signal combinations include a total of at most about 1, at most about 2, at most about 3, at most about 4, at most about 5, at most about 6, at most about 7, at most about 8, at most about 9, at most about 10, at most about 15, at most about 20, at most about 25, or fewer different detectable signals (e.g. fluorescence channels).

The plurality of nucleic acid molecules of the nucleic acid sample identified may comprise multiple copies of the same loci. For example, 1,000 nucleic acid molecules corresponding to a target loci may be identified. In some cases, the plurality of nucleic acid molecules may comprise one or more mutations relative to a reference sequence. In some cases, the plurality of nucleic acid molecules of the nucleic acid sample identified may comprise at least about 1,000, at least about 5,000, at least about 10,000, at least about 50,000, at least about 100,000, at least about 500,000, at least about 1,000,000, at least about 2,000,000, at least about 3,000,000, at least about 4,000,000, at least about 5,000,000, at least about 6,000,000, at least about 7,000,000, at least about 8,000,000, at least about 9,000,000, at least about 10,000,000, at least about 15,000,000, at least about 20,000,000, at least about 25,000,000, at least about 30,000,000, at least about 35,000,000, at least about 40,000,000, at least about 45,000,000, at least about 50,000,000, at least about 55,000,000, or more nucleic acid molecules. In some cases, the plurality of nucleic acid molecules of the nucleic acid sample identified may comprise at most about 1,000, at most about 5,000, at most about 10,000, at most about 50,000, at most about 100,000, at most about 500,000, at most about 1,000,000, at most about 2,000,000, at most about 3,000,000, at most about 4,000,000, at most about 5,000,000, at most about 6,000,000, at most about 7,000,000, at most about 8,000,000, at most about 9,000,000, at most about 10,000,000, at most about 15,000,000, at most about 20,000,000, at most about 25,000,000, at most about 30,000,000, at most about 35,000,000, at most about 40,000,000, at most about 45,000,000, at most about 50,000,000, at most about 55,000,000, or fewer nucleic acid molecules. In some cases, the plurality of nucleic acid molecules of the nucleic acid sample identified may comprise about 1,000-55,000,000, about 5,000-50,000,000, about 10,000-45,000,000, about 50,000-40,000,000, about 100,000-35,000,000, about 500,000-30,000,000, about 1,000,000-25,000,000, about 2,000,000-20,000,000, about 3,000,000-15,000,000, about 4,000,000-10,000,000, about 5,000,000-9,000,000, or about 6,000,000-8,000,000 nucleic acid molecules. In some cases, the plurality of identified nucleic acid molecules of the nucleic acid sample may comprise at least 10,000 nucleic acid molecules. In some cases, the plurality of identified nucleic acid molecules of the nucleic acid sample may comprise at least 100,000 nucleic acid molecules. In some cases, the plurality of identified nucleic acid molecules of the nucleic acid sample may comprise at least 1,000,000 nucleic acid molecules.

The identified plurality of nucleic acid molecules of the nucleic acid sample may comprise one or more loci. For example the identified plurality of nucleic acid molecules of the nucleic acid sample may comprise sequences corresponding to three loci. The loci may comprise sequences associated with a gene, a mutation, a genomic region of interest, a target nucleic acid sequence, or a combination thereof. The plurality of identified nucleic acid molecules of the nucleic acid sample may comprise at least about 1, at least about 2, at least about 3, at least about 4, at least about 5, at least about 6, at least about 7, at least about 8, at least about 9, at least about 10, at least about 12, at least about 14, at least about 15, at least about 16, at least about 18, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more different loci. The plurality of identified nucleic acid molecules of the nucleic acid sample may comprise at most about 1, at most about 2, at most about 3, at most about 4, at most about 5, at most about 6, at most about 7, at most about 8, at most about 9, at most about 10, at most about 12, at most about 14, at most about 15, at most about 16, at most about 18, at most about 20, at most about 25, at most about 30, at most about 35, at most about 40, at most about 45, at most about 50, at most about 55, at most about 60, at most about 65, at most about 70, at most about 75, at most about 80, at most about 85, at most about 90, at most about 95, at most about 100, or fewer different loci. The plurality of identified nucleic acid molecules of the nucleic acid sample may comprise about 1-100, about 2-95, about 3-90, about 4-85, about 5-80, about 6-75, about 7-70, about 8-65, about 9-60, about 10-55, about 12-50, about 14-45, about 15-40, about 16-35, about 18-30, or about 20-25. The plurality of identified nucleic acid molecules of the nucleic acid sample may comprise at least 10 different loci. The plurality of identified nucleic acid molecules of the nucleic acid sample may comprise at least 20 different loci. The plurality of identified nucleic acid molecules of the nucleic acid sample may comprise at least 30 different loci.

The methods described herein may be completed in a period of time. In some cases, imaging the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, reacting the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, generating the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, generating and reacting the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, reacting and imaging the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, generating, reacting, and imaging the plurality of partitions may be completed in no more than about 5 minutes, no more than about 10 minutes, no more than about 15 minutes, no more than about 20 minutes, no more than about 25 minutes, no more than about 30 minutes, no more than about 45 minutes, no more than about 1 hour, no more than about 2 hours, no more than about 3 hours, no more than about 4 hours, no more than about 5 hours, no more than about 6 hours, no more than about 7 hours, no more than about 8 hours, no more than about 9 hours, no more than about 10 hours, or longer. In some cases, reacting, and imaging the plurality of partitions may be completed in no more than about 3 hours.

In relation to generation of stabilized partitions having suitable clarity (e.g., with or without refractive index matching), the method(s) described herein may include transmission of signals from individual stabilized partitions from within the closed collecting container, for readout (e.g., by an optical detection platform, by another suitable detection platform). In some embodiments, clarity may be defined in units associated with clarity or turbidity (e.g., NTU, FNU). In some cases, the threshold level of clarity can be measured for the emulsion(s) generated according to the methods described. In one variation, clarity may be characterized in relation to transmissivity as detectable by a transmission detector and/or in relation to a suitable distance or depth (e.g., depth or distance into a collecting container for the emulsion; through a depth of a container of the emulsion, along an axis in which measurement of clarity is performed, etc.). In some cases, the threshold level of clarity of the stabilized partitions may be associated with a transmissivity greater than 70% transmissivity, greater than 80% transmissivity, greater than 90% transmissivity, greater than 95% transmissivity, greater than 99% transmissivity, etc. In some cases, upon measuring clarity of the stabilized partitions using a transmission detector the stabilized partitions may be characterized by a clarity associated with greater than 70% transmissivity, greater than 80% transmissivity, greater than 90% transmissivity, greater than 95% transmissivity, greater than 99% transmissivity, etc. In some cases, the clarity may be above the threshold level of clarity.

In some cases, during storage and/or incubation, as discussed above, clarity of stabilized partitions within the closed collecting container may regress to a less clear state (e.g., from over 80% transmissivity to less than 80% transmissivity). In some cases, collecting container comprising the plurality of partitions may be centrifuged (e.g., re-centrifuged). In some cases, centrifuging (e.g. re-centrifuging) the container comprising the plurality of partitions may improve and/or restore clarity of the stabilized partitions (e.g., to an over 80% transmissivity format, to an over 80% transmissivity format, etc.).

### Computer Systems

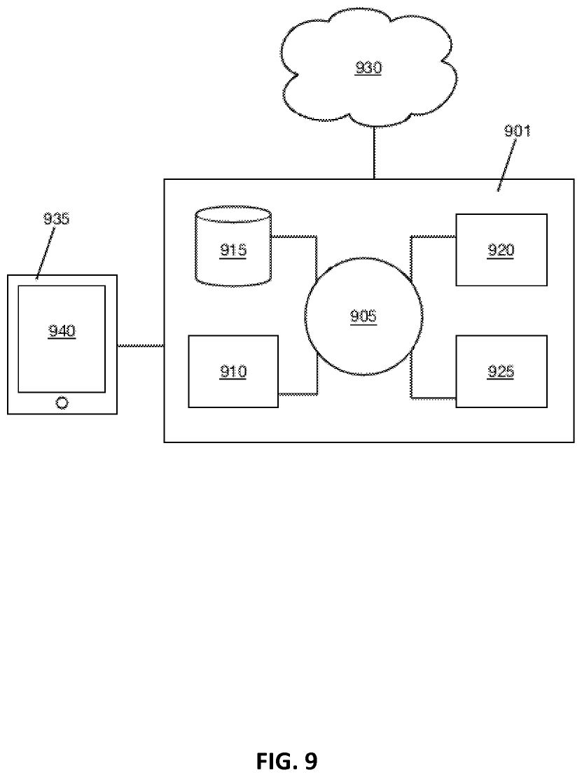

The present disclosure provides computer systems that may be programmed to implement methods of the disclosure. FIG. 9 shows a computer system 901 that is programmed or otherwise configured to, for example, perform a digital multiplexed analysis of a sample distributed across a set of partitions, where targets may be tagged using one or more multiplexing strategies described (e.g., involving color combinatorics, involving stimulus-responsive probes, involving tandem probes, involving probes that exhibit FRET behavior, with combinations of probes, with permutations of probes, etc.). Performing the digital multiplex analysis can include: reacting the sample with the set of processing materials within the set of partitions, and detecting signals from the set of partitions upon performing 3D scanning of the set of partitions with a number of color channels. In relation to color combinatorics and other multiplexing strategies described, signals from specific targets can correspond with a set of color combinatorics paired with targets of a set of targets potentially represented in the sample and contained within partitions of the set of partitions, and the set of targets can have a total number greater than the number of color channels used to detect colors corresponding to the set of color combinatorics. The digital multiplexed analysis can have SNR performance achievable as described.

The computer system 901 can additionally or alternatively perform other aspects of digital multiplexed assays for characterizations involving other loci of interest, with applications of use described above.

The computer system 901 can regulate various aspects of analysis, calculation, and generation of the present disclosure, such as, for example, generating a plurality of partitions (e.g., from an aqueous mixture including sample material and materials for an amplification reaction) within a collecting container at a desired rate, transmitting heat to and from the plurality of partitions within the collecting container, performing an optical interrogation operation with the plurality of partitions within the collecting container, and/or performing one or more digital multiplexed assay steps. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

In some embodiments, the network 930 is a telecommunication and/or data network. The network 930 can include one or more computer servers, which can enable distributed computing, such as cloud computing. For example, one or more computer servers may enable cloud computing over the network 930 (“the cloud”) to perform various aspects of analysis, calculation, and generation of the present disclosure, such as, for example, generating a plurality of droplets within a collecting container at a predetermined rate or variation in polydispersity. Such cloud computing may be provided by cloud computing platforms such as, for example, Amazon Web Services (AWS), Microsoft Azure, Google Cloud Platform, and IBM cloud. In some embodiments, the network 930, with the aid of the computer system 901, can implement a peer-to-peer network, which may enable devices coupled to the computer system 901 to behave as a client or a server.

The CPU 905 may comprise one or more computer processors and/or one or more graphics processing units (GPUs). The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some embodiments, the circuit is an application specific integrated circuit (ASIC).

The storage unit 915 can store files, such as drivers, libraries and saved programs. The storage unit 915 can store user data, e.g., user preferences and user programs. In some embodiments, the computer system 901 can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine-readable code can be provided in the form of software. During use, the code can be executed by the processor 905. In some embodiments, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 901, can be embodied in programming. Various aspects of the technology may be thought of as “products” or “articles of manufacture” and may be in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, a visual display indicative of stages of or results from performing a digital multiplexed analysis of a sample distributed across a set of partitions, where targets are tagged using one or more multiplexing strategies described (e.g., involving color combinatorics, involving stimulus-responsive probes, involving tandem probes, involving probes that exhibit FRET behavior, with combinations of probes, with permutations of probes, etc.). Performing the digital multiplex analysis can include reacting the sample with the set of processing materials within the set of partitions, and detecting signals from the set of partitions upon performing 3D scanning of the set of partitions with a number of color channels. The UI 940 can additionally or alternatively be adapted for performing other digital assays involving other loci of interests and/or other calculations, as described. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, generate a plurality of droplets within a collecting container with desired characteristics.

### EXAMPLES

### Example 1

In this example, patient samples were analyzed for detection of the KIT D816V mutation.

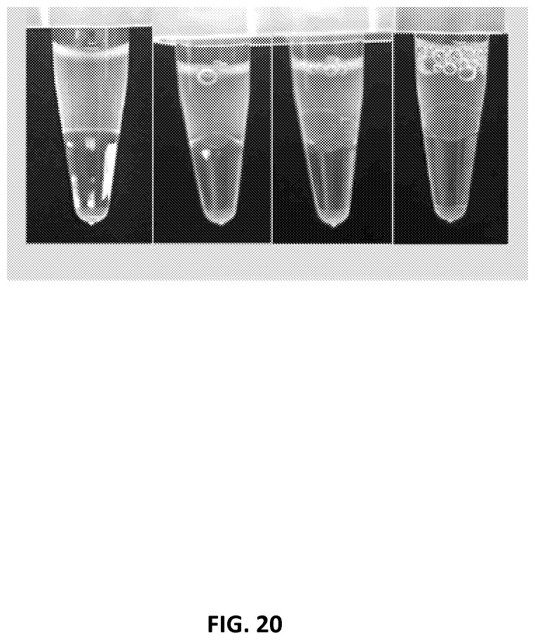

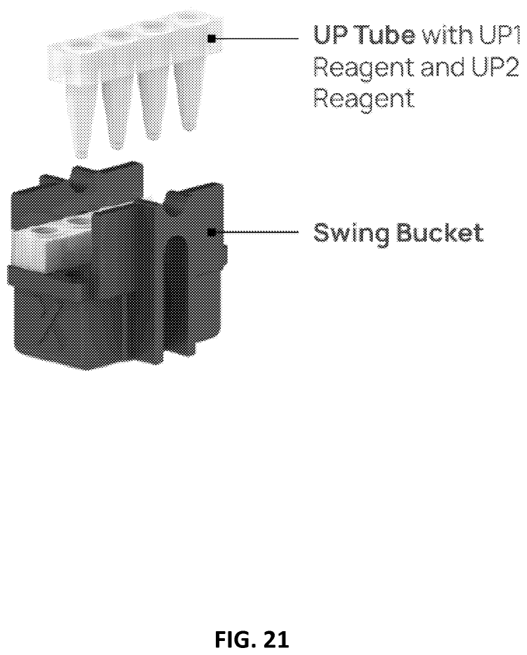

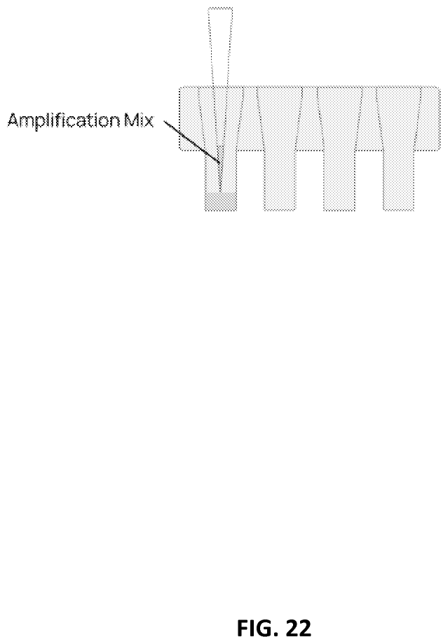

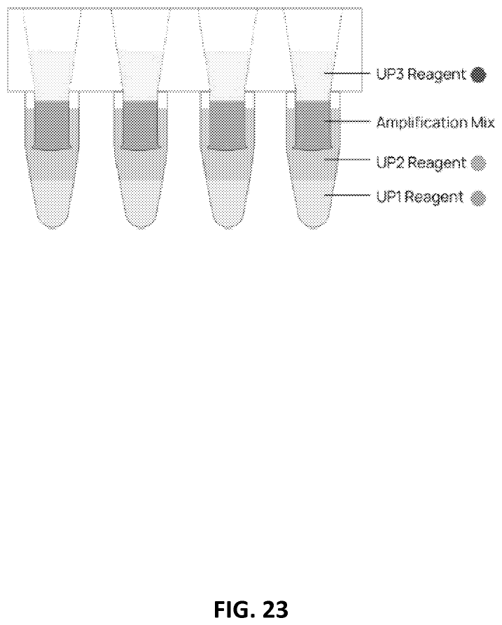

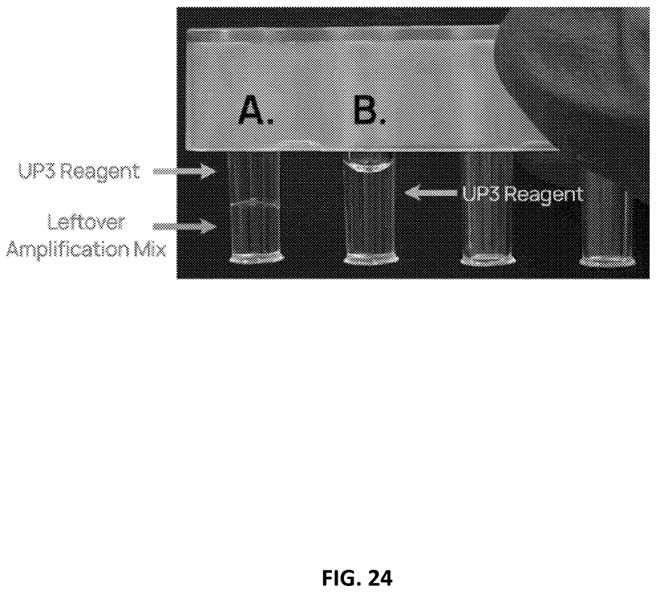

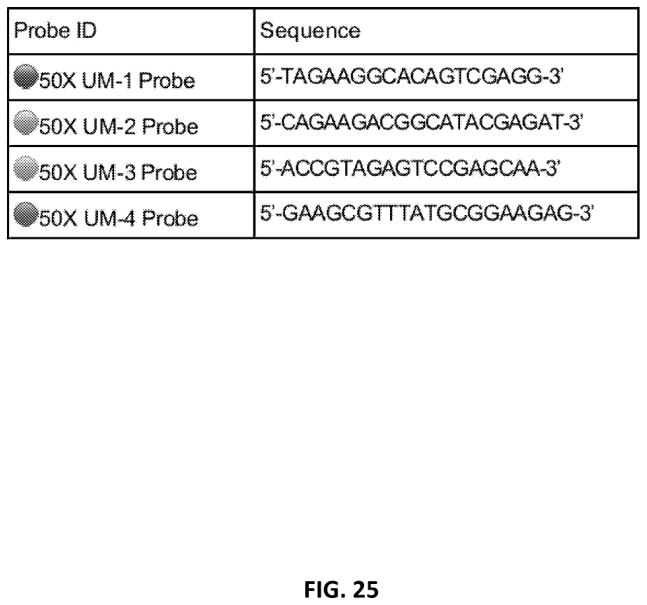

Blood samples or bone marrow samples from 16 patients were processed using EDTA extraction to isolate DNA. For each patient sample, between 0.15-1.452 μg of isolated DNA was added to a 50 μl reaction solution. Each reaction solution also contained materials to perform qPCR reactions, including a polymerase, deoxynucleotides, primers, and buffer. The primers were designed to be able to detect the KIT D816V mutation and differentiate between the KIT D816V mutation and wildtype alleles. The amplification primers were the same for detecting the D816V mutation and wildtype allele, whereas the hydrolysis probe was different for the KIT D816V mutation compared to the wildtype allele. A plurality of partitions was generated for each 50 μl reaction solution using centrifugation. Briefly, for each sample, fluid layers of an aqueous solution and an immiscible solution with the aqueous solution were generated in a microcentrifuge tube (FIG. 20). The aqueous solution was layered below the immiscible solution. A spin column was prepared for the centrifugation reaction by adding a layer of immiscible solution to the top above a membrane. The 50 μl reaction solution was then added to the spin column as a layer beneath the immiscible solution layer (FIG. 22). The spin column was placed within the microcentrifuge layer according to the diagram in FIG. 23. In this case, UP3 and UP2 denote the immiscible solutions, ‘Amplification Mix’ denotes the 50 μl reaction solution, and UP1 denotes the aqueous solution within the microcentrifuge tube. The microcentrifuge tubes with the spin columns were then placed in a swing bucket centrifuge using the holder shown in FIG. 21. The samples were then centrifuged resulting in the formation of the plurality of partitions for each patient sample. Confirmation that the plurality of partitions were formed correctly was done by visual inspection of the spin columns after centrifugation. A visible layer of aqueous solution within the spin column after centrifugation may be indicative that some of the 50 μl reaction solution was not contained within the plurality of partitions formed. Each plurality of partitions comprised at least 30 million partitions.

The plurality of partitions for each patient sample were then subjected to rounds of heating to perform a PCR reaction. After performance of the PCR reaction, partitions that contained the target nucleic acids with the KIT D816V mutation produced positive signals, based on the probes originally added to the 50 μl reaction solution, which included a fluorescent modification that was released upon subjecting the plurality of partitions to the amplification reaction conditions. The plurality of partitions were imaged using a custom light sheet microscope. Signals associated with partitions exhibiting a detectable signal were measured and analyzed to assess the sensitivity and specificity of the assay. Results generated from the described workflow were analyzed in comparison to a commercially-available digital PCR assay. The commercially-available digital PCR assay was not performed by generating the plurality of partitions, according to the workflow described above. Instead, 3 different samples having 100 ng of input DNA each were distributed across 78,000 droplets. Positive signals from these samples were detected and compared to those generated from the plurality partitions previously described with 30 million partitions per patient sample. There was 90.9% concordance in detecting positive signal, between the results generated using the workflow described above, in relation to the workflow of the commercially-available digital PCR assay, with an associated p-value of 0.11. Additionally, the results from analyzing patient samples across 30 million partitions as described were compared to control samples comprising a synthetic construct comprising the KIT D816V mutation. For the analysis involving control samples, 10,026 wild type partitions were detected compared to 5 mutation containing partitions. Finally, a patient sample was used in a dilution experiment where input DNA was added at known amounts of 300 ng-1000 ng DNA. Analysis of these samples relative to expected detection of the KIT D816V mutation showed 100% concordance.

### Example 2

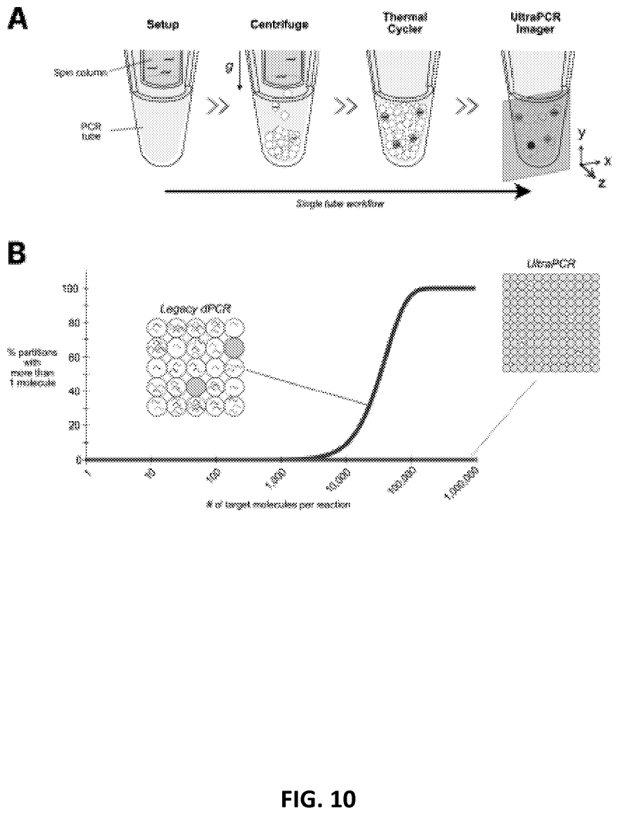

The example described herein demonstrates how singlet amplification using UltraPCR leads to clean signal amplification and detection (FIG. 10A), which enables high order multiplexing and a straightforward data analysis workflow. First, examples of the emulsion and imaging systems unlock the ability to incorporate 10+ fluorescent dyes into a single PCR assay. Second, singlets enable combinatorial labeling without the statistical limitations of multiple target occupation seen in other dPCR technologies (which may comprise deconvolution, use of correction factors, or a combination thereof). Third, the quasi-solid phase optically clear emulsion pellet allows for repeated interrogation via 3D imaging of the large number of partitions. When combined, these features allow for a highly scalable, inexpensive approach to high-level PCR multiplexing. The example demonstrates that these methods can comprise high-plex, high-precision assays by showcasing an 8-color, 22-plex pathogen ID panel.

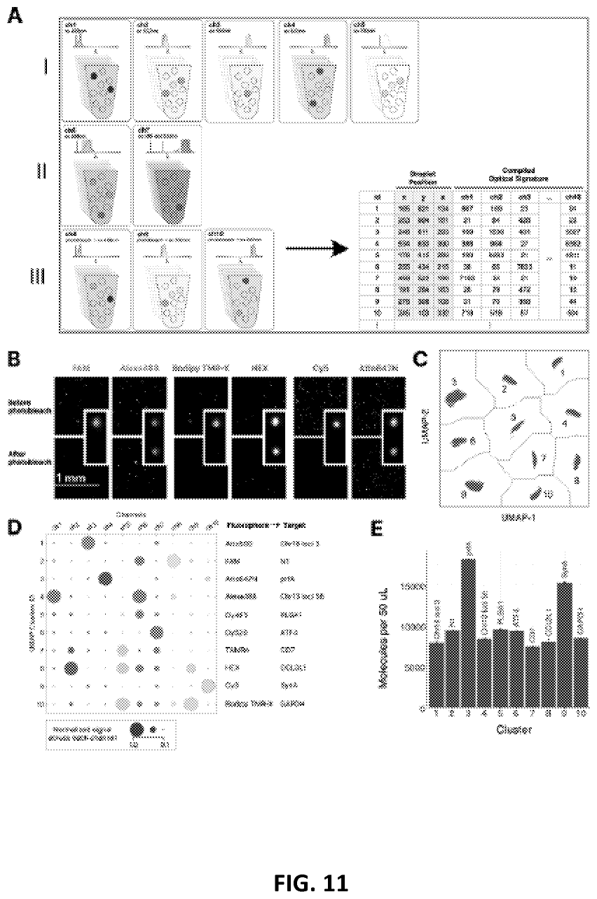

Optical signature profiling unlocks additional fluorescent dye repertoire for PCR. When applying centrifugation-based partitioning, each DNA target molecule of a sample is randomly partitioned into individual singlets directly into the PCR tube (FIG. 10A). Collectively, the unique chemistry of the example renders the resulting emulsion optically clear with spatially immobilized droplets/partitions (with immobilization resilient against movement of the container), facilitating the use of 3D light sheet microscopy or other 3D scanning techniques to measure fluorescence signals of individual partitions in a massively parallel manner with high spatial resolution (FIG. 10A). A single light sheet channel may take about 40 seconds to sweep through a sample of ~34 million partitions, and lower times can be achieved. The static state of the emulsion enables repeated imaging where the 3D spatial location of partitions remains traceable without sample manipulation. Leveraging these features, UltraPCR can perform serial imaging of the same PCR tube with different “channels”—each channel having its own excitation and emission configuration—to generate an “optical signature” of each positive partition (FIG. 11A).

The concept of an optical signature enables the profiling of highly similar fluorescent dyes that may otherwise be difficult to distinguish in other PCR platforms. For example, HEX and TAMRA dyes may not use concurrently in qPCR/dPCR due to their high spectral overlap. However with the example of UltraPCR described herein, these dyes can be differentiated across serial imaging steps. Using the same excitation laser (532 nm), the example involved creation of 2 channels in UltraPCR that employ different emission band passes for the detection of HEX (ch2) and TAMRA (ch5) (FIG. 11A). Even though the emission of HEX and TAMRA dyes are detectable in both channels, their fluorescence intensities in each channel collectively render distinct optical signatures.

The example further investigated and incorporated use of a large number of dyes. Methods employed with the example distinguished three major dye classes for a total of 10 dyes that are compatible with the UltraPCR Imager configuration (FIG. 11A) of the example, using different combinations of excitation lasers, emission filters, and imaging settings (also referred to as “channels”). The first class of dyes, include Alexa488, HEX, Atto590, Atto647N, and TAMRA (ch1, ch2, ch3, ch4, ch5, respectively).

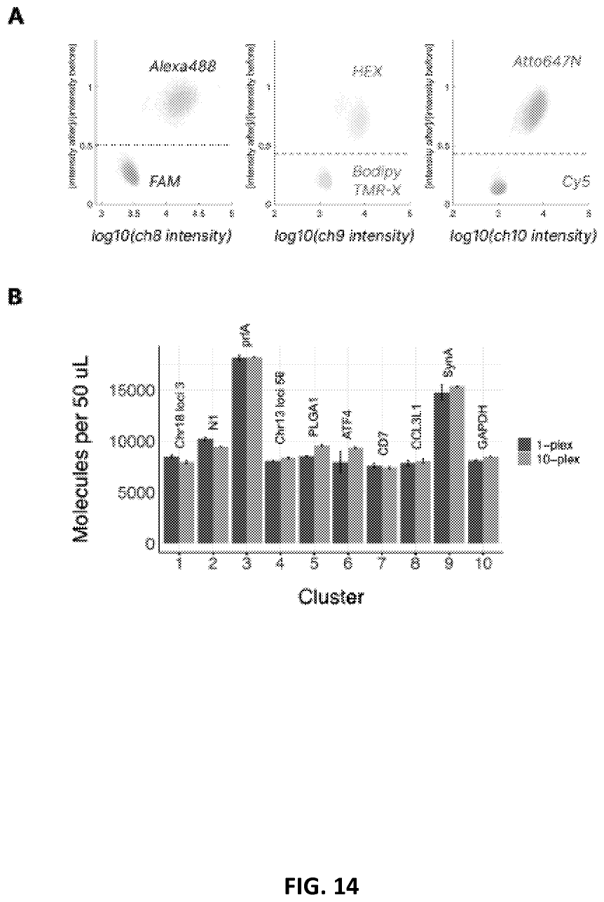

The class II dyes (Dy485XL and Dy520XL) use the same excitation lasers as the class I, but because they have larger Stokes shifts, the optical signatures can be uniquely identified by creating channels with the same excitation laser but different emission filters (ch6, ch7). The class III dyes are differentiable based on their photobleaching properties. The example further incorporated use of 3 additional dyes that have similar optical signatures as class I dyes, but can be photobleached in a controllable manner. Light sheet images of positive partitions labeled with these two classes of dyes reveal their distinctive optical signature (FIG. 11B); these class I and III dye pairs are FAM (photo bleach sensitive, ch8) vs Alexa488, Bodipy TMR-X (photo bleach sensitive, ch9) vs HEX, and Cy5 (photo bleach sensitive, ch10) vs Atto647N. The method incorporates a photo bleach event using UltraPCR Imager light sheet between 2 imaging events of the same excitation/emission setting that allows for differentiation between these dye pairs (FIG. 14A).

This example processes a 10-plex UltraPCR panel using a dye differentiating approach and the 10 dyes described. Positive partitions for each channel were automatically detected by signal intensity difference from background using methods described and executed by the UltraPCR Imager. Optical signatures from 10 channels were gathered to generate a vector of intensity values [ch1-ch10] for each positive partition detected in different locations [x,y,z] of the tube (FIG. 11A). This 2D matrix was then used to visualize partitions using UMAP, followed by clustering to distinguish the targets (FIG. 11C). Using these techniques provided rapid and clear distinguishing of 10 clusters of positive partitions with similar optical signatures in an unsupervised manner, and without overlap between clusters. The clear separation of clusters in UMAP showcases the difference in optical pattern of partitions with different dyes. The optical signature of each cluster was identified by measuring median signals of partitions residing in them to identify the fluorophore (and thereby the amplicon target) represented by the cluster (FIG. 11D). The data shown for each channel is normalized by the maximum signal across each channel. Since each datapoint was represented by a single target molecule, the molecule count per target was simply the number of datapoints per cluster (FIG. 11E). The molecule counts per cluster in the 10-plex assay matched the expected counts based on a single plex assay with no observable differences in counting precision (FIG. 14B). We were able to demonstrate that singlet partitions paired with optical signature profiling can increase multiplex capacity in PCR.

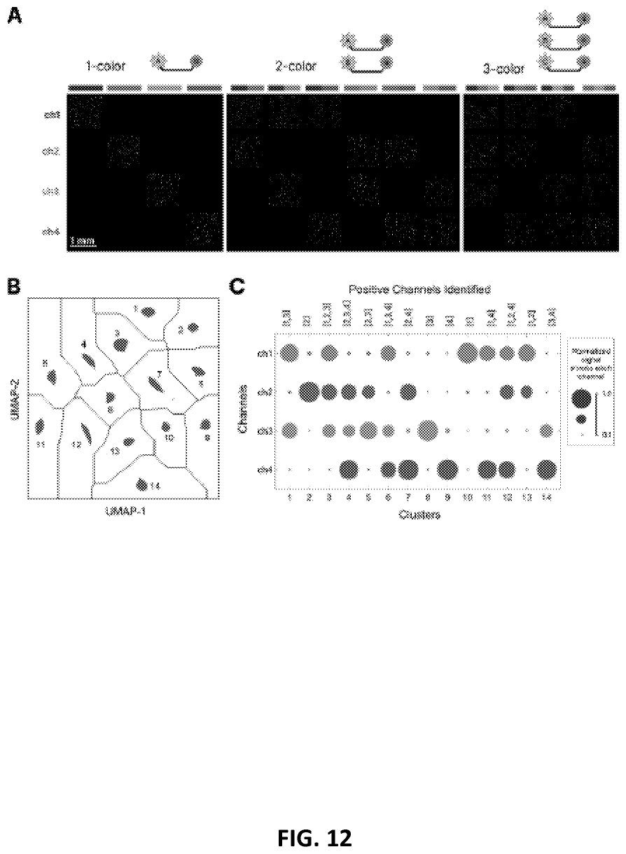

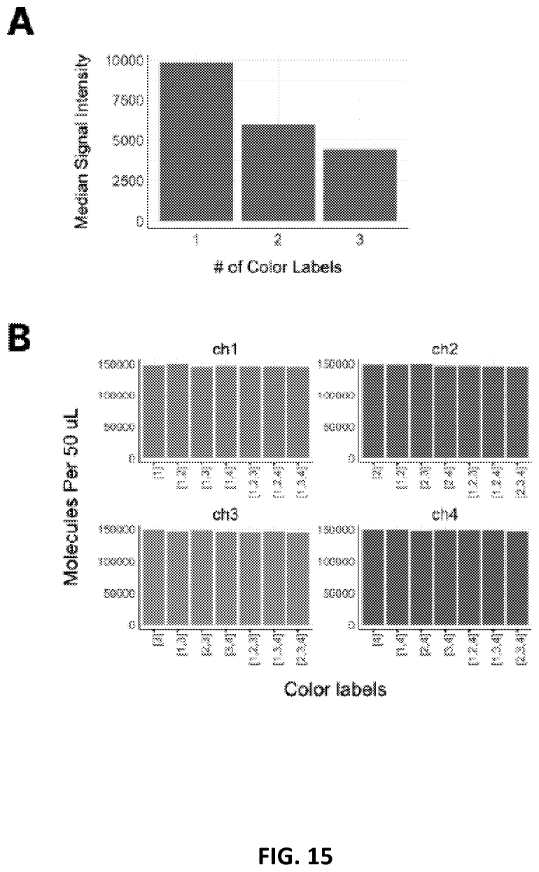

The multiplex capacity can be extended when dyes are combined combinatorially, as described (e.g., “comboplexed”) creating new distinctive optical signatures. The method labeled a single target with either 1, 2, or 3 fluorescent labels (although more labels can be implemented) where each configuration targets the same gene using the same probe sequence but conjugated to different dyes (FIG. 12A). Using a set of 4 dyes, all 14 combinations were tested, where each comboplex assay was serially imaged in 4 channels (ch1-4) to collect fluorescent intensities (FIG. 12A). In this particular configuration of the assay, even though the fluorescence intensity of targets with more than one fluorophore decreased due to probe competition for the same target amplicon the method was able to call positive partitions apart from background (FIG. 15A). The method first analyzed positive partitions in each channel independently and saw that the molecule counts for all label combinations were highly comparable (FIG. 15B). Another way of analyzing comboplex data was to combine the fluorescence optical signatures into a multivariable matrix and use UMAP and automatic clustering to identify dye combinations (FIG. 12B). The separation of the clusters with combinatorial labels is comparable with the separation of clusters with single dye per targets (FIG. 11C) indicating efficacy of comboplex assays. Each of the 14 clusters identified in the UMAP also have unambiguous optical signatures that can be used to map the actual dye combination used (FIG. 12C).

The concept of comboplex complements the singlet optical signature technique, as the same set of “base” fluorescent labels can be used to massively expand the configurations for single molecule labeling. The number of comboplex targets can be calculated by the following equation: P (n, k)=n!/(n−k)!, where n is the number of fluorophores available for the platform, and k is the number of labels used per target. Combined with the possibility that a target can be labeled with 1 or 2 fluorophores (k=1 or 2), the upper limit of comboplex can be extremely high. This approach is particularly useful because UltraPCR has at least a 10 dye compatibility as demonstrated above. Therefore, the theoretical plex can reach >50 if 1 and 2 dye combinations are used, which is at least 10-fold higher than other PCR assays, as outlined in Table 1.

TABLE 2
Possible number of multiplex combinations
achievable by the comboplex approach.

| # of | | | | k = 1, |
|---|---|---|---|---|
| dyes (n) | k = 1 | k = 2 | k = 3 | 2 or 3 |
| 4 | 4 | 6 | 4 | 14 |
| 5 | 5 | 10 | 10 | 25 |
| 6 | 6 | 15 | 20 | 41 |
| 7 | 7 | 21 | 35 | 63 |
| 8 | 8 | 28 | 56 | 92 |
| 9 | 9 | 36 | 84 | 129 |
| 10 | 10 | 45 | 120 | 175 |

Demonstration of simultaneous digital counting of 22 targets in a single tube

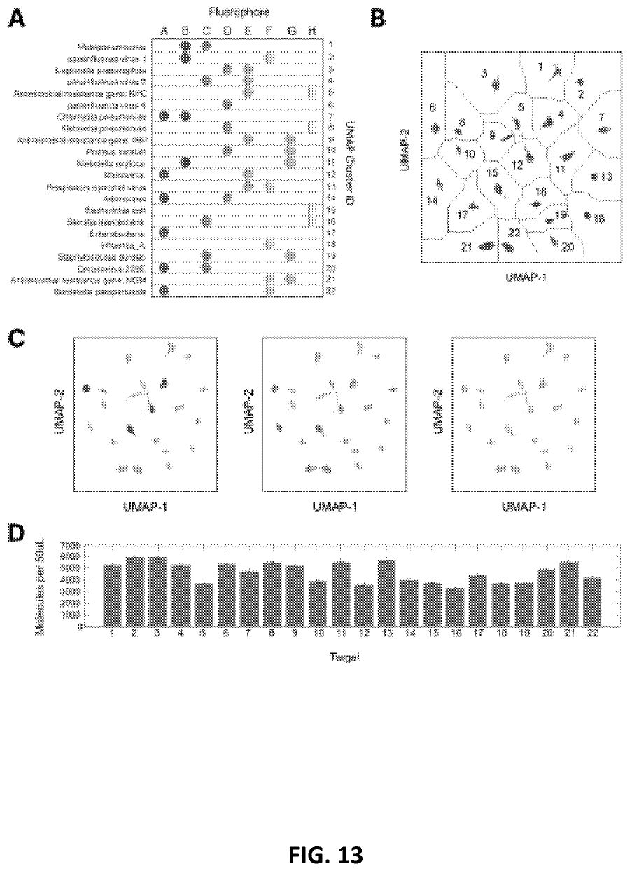

The example provides an expanded dye repertoire to build a proof-of-concept comboplex panel in UltraPCR. The example was applied to a set of 22 common targets for identifying respiratory pathogens and associated antibiotic resistance genes. In the multiplex design, 8 available DNA dyes were used to label each target with either 1 or 2 unique labels using a Universal Multiplex strategy (FIG. 13A). These multiplex primers were designed in silico using a custom primer design algorithm with the principles to minimize primer dimers and non-specific primer extensions to maximize compatibility. Each target labeling strategy was confirmed in single target reactions before pooling together for multiplexing.

With this magnitude of multiplex capability, the example implemented a new analysis workflow to streamline data interpretation. The method generated a ‘reference optical signature’ for each of the 22 targets by performing the 22-plex UltraPCR assay on spike-in DNA of each target individually. Each of these reactions was imaged through 8 channels to record the collective optical signature for each target. All optical signatures were combined and the 8-dimensional data was projected onto a 2D graph using UMAP. Twenty-two distinct clusters were identified using an automatic clustering algorithm (FIG. 13B). Using this automated approach, the method avoids tedious manual determination of target assignment for each partition, a method still commonly applied in legacy systems. Note that this map of the 22-plex assay can be applied across multiple experiment days and probe batches, further streamlining the analysis of these higher order multiplexing samples.

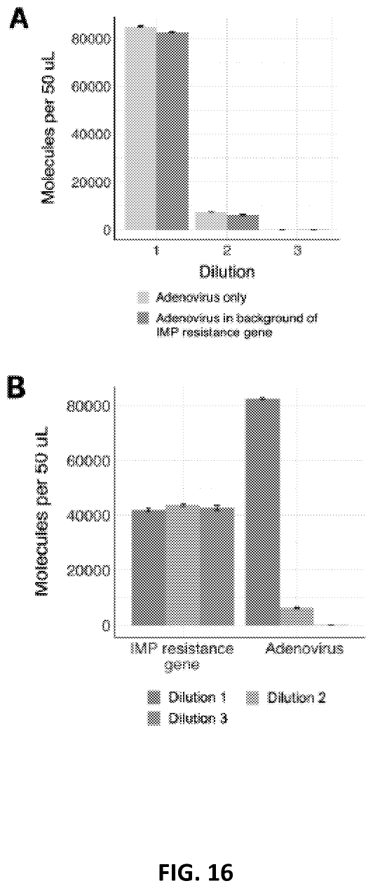

The method uses this reference map to determine the identities and molecule counts of targets in samples. In a first implementation, 2 targets were added concurrently at different concentrations; in target 1, Imp, was spiked at a constant level whereas target 2, Adv54, was serially diluted. Each reaction was analyzed independently using the “cluster-counting” workflow aforementioned. The method returned results showing that even at wide variation in spike-in levels of these 2 targets, the precision of molecule counting by UltraPCR remains extremely high (FIG. 16). The data is consistent with the notion that even at such a high order of “multiplexing”, UltraPCR remains functional at a singlet realm where amplification and detection remain independent between different target molecules.

The method further extended the singlet multiplexing notion by spiking in more targets concurrently into a 22-plex comboplex assay. The method tested replicates where 4, 8, or all 22 target templates were added into the assay; the optical signature of each positive partition was overlaid with the reference map to show little batch-to-batch variability in cluster identification, even though this spike-in experiment was performed on a separate day using a separate batch of fluorescent probes for some targets. In all conditions, the method was able to simultaneously detect the presence of multiple targets and quantify the targets (FIG. 13C). Additionally, for the 22-target test, the 4 technical replicates showed a remarkably high counting precision (FIG. 13D).

UltraPCR utilizes millions of picolitre partitions for PCR analysis. Each sample is divided into millions picolitre-sized partitions (e.g. more than 30 million partitions) using a simple centrifugation step (FIG. 10A)—a 1500-fold higher number of partitions than legacy dPCR. At this magnitude, the ratio of DNA to partition is so small that Poisson statistics indicate that target molecules are individually distributed into separate partitions, giving rise to single molecule (e.g. target molecule) partitions—or singlets—in a massively parallel manner (FIG. 10B). In this realm, PCR within each partition amplifies a single template molecule without competition. Fundamentally, UltraPCR is similar to single molecule PCR demonstrated via limiting dilution, but in a massively parallel manner such that even up to 1 million target molecules can be individually partitioned and analyzed, in some cases without the use of Poisson correction (FIG. 10B). The precision and dynamic range of UltraPCR is greater than the capability of legacy dPCR (FIG. 10B) that challenging assays such as fetal aneuploidy detection become possible with as low as 4% trisomy DNA against the backdrop of a normal individual's background DNA. This provides a PCR platform achieving NGS-like counting precision. The advantages of singlet partitioning, however, extends beyond counting capabilities; in this study, we show that singlet partitioning itself simplifies the biochemistry of multiplex PCR, enabling a new era towards straightforward and higher-order multiplexing.

In both bulk qPCR and dPCR regimes, partitions may contain mixed targets due to limited partition number (n=1 for qPCR, and n~20,000 for dPCR) (FIG. 10B). Each dye assigned per target therefore may be distinct by design with minimal spectral overlap. Due to this limitation, other dPCR platforms may support 2-6 colors. Even at this low order of multiplexing, emission crosstalk between fluorophores may comprise various compensation techniques to remove false positive fluorescence signals. In addition to a low number of concurrently usable fluorophores for multiplex PCR, the amplification signal may be extremely high such that the biomarker signal rises above what is removed during compensation. The UltraPCR platform reimagines how dyes can be used for multiplexing by leveraging our method of optically profiling singlet partitions. The resulting optical signature data is comparable to gene expression analysis in single cells—where each partition can have fluorescence expression across many imaging channels—and can be collectively analyzed like NGS datasets (FIG. 11). The proof-of-concept datasets were processed with multivariable analytic tools such as UMAP and associated clustering techniques, showcasing how future PCR analysis of many biomarkers can be just as easily automated, with minimal manual intervention for high throughput, high-plex assays (FIGS. 11-13).

The example can be extended to expand the number of dyes that can be used concurrently in a PCR reaction. The repertoire of dyes that can be used in UltraPCR can be further expanded by at least 2 ways: 1) increasing the number of lasers from 4 to a higher number, matching that of high-end flow cytometers, 2) expanding the number of dyes (available and/or custom) that can be conjugated to DNA probes, or 3) a combination thereof. Consider the number of lasers and dyes observed in flow cytometry: as many as 9 lasers and 50 targets (or parameters) have been commercially achieved.

UltraPCR-enabled singlet amplification overcomes the many challenges with PCR multiplexing and increases the level of multiplexing. By having an expanded dye portfolio (that can be further expanded) and combinatorial labeling enabled by singlet realm, development of a 22-plex pathogen ID panel for the detection of common respiratory viruses and antibiotic resistance genes (FIG. 13) was achieved. The maximum multiplex capacity is much higher than 22; when utilizing all 10 dyes, where each target is labeled with either 1 or 2 dyes, the maximum limit may be 55-plex (Table 1).

The methods described herein can be applied to many applications, ranging from basic research, translational research, to clinical diagnostics. UltraPCR's multiplexing capability can be useful in gene expression studies and biomarker discoveries, where panels of biomarkers identified by microarray or RNA-seq studies can be quickly and simultaneously validated with a PCR based readout with high precision, reducing the amount of sample usage and number of reactions that may be used. In infectious disease diagnostics, the ability to accurately quantitate one or more (in co-infection cases) pathogens with UltraPCR allows clinicians to determine which pathogen(s) may be present, providing potentially actionable results for quick, targeted treatment selection. In comparison, microbiology culture techniques may take several days longer in turnaround time with no quantitative result on pathogen level. In oncology, UltraPCR can be used to displace certain amplicon sequencing panels, providing broader patient access while dramatically reducing turnaround time and cost of each test as compared to NGS. The additional workflow advantages of UltraPCR—zero dead volume and high PCR loading volume (>30 μL)—may be particularly beneficial in liquid biopsy and rare molecule detection applications.

### Materials and Methods

UltraPCR Workflow: All UltraPCR reaction mixes were prepared using 4× UltraPCR mix. For every sample, 50 μL of the UltraPCR reaction mix (with primers, probes, and DNA template) was added to the UltraPCR Spin Columns, outfitted into PCR strip tubes carrying emulsifying reagents. The strip assembly was loaded into a custom UltraPCR swing bucket for use in a centrifuge. Up to 48 samples were spun for 20 min at 16,000 g to form UltraPCR emulsions. After centrifugation, the spin columns were discarded, and the PCR tubes containing the emulsions were sealed and placed into a thermal cycler. The same PCR strip tubes were then placed into an UltraPCR Imager for positive partition scanning, where a laser light sheet was translated across the PCR tube and the illuminated partitions were imaged. Four lasers were utilized to scan up to 10 dyes, where each dye had an optimized imaging setting with defined excitation laser(s) and emission filters. The current configuration of the UltraPCR Imager includes excitation wavelengths at 488 nm, 532 nm, 594 nm, and 640 nm.

For each sample, cross sectional images of the PCR tube containing emulsions collected by light sheet imaging were compiled to generate a 3D reconstruction of the PCR tube to count positive partitions using custom software. Given that signal from UltraPCR represents single molecules, the UltraPCR Imager analysis pipeline is deployed without user intervention for positive partition identification. In all samples, an auto-thresholding algorithm was applied to gate signal that is DNA-positive versus negative. Multivariable analysis was performed, using a UMAP package for visualization, and architecture for clustering.

Samples: Genomic RNA materials for Influenza A, Influenza B and SARS Covid 2 (SC2) were obtained and Jurkat total RNA was obtained. cDNA was synthesized using. Synthetic templates were obtained as gBlocks. All serial dilutions and template dilutions used TE buffer to reach intended copy numbers and confirmed using UltraPCR prior to multiplex experiments.

Assay Designs: Unless specified, individual primers and probes were designed using Primer3. For multiplex assays, primer candidates designed by Primer3 were screened and selected for the multiplex panel using a custom algorithm with the principles to minimize primer dimers and non-specific primer extensions to maximize compatibility. Sequences of primers and probes and its conditions are shown in Table S1.

| | Stage 1 | Stage 2 | Stage 3 |
|---|---|---|---|
| Assay | (1 cycle) | (40 cycles) | (1 cycle) |
| Hydrolysis | 95° C., | 95° C., 20 s | Hold at |
| probes | 2 min | 55° C., 30 s | 12° C. |
| | | 68° C., 30 s | |
| Universal | 95° C., | 95° C., 20 s | Hold at |
| Multiplex | 2 min | 56*° C., 60 s | 12° C. |
| (UM) | | 95° C., 30 s | |

Fluorophore characterization: Ten fluorophores were tested in the experiments, including 3 pairs of fluorophores with similar excitation/emission spectra (FAM vs Alexa 488, Bodipy TMR-X vs HEX, Cy5 vs Atto 647N), 2 large stoke shift fluorophores (Dy-485XL, Dy-520XL), TAMRA, and Atto590. TaqMan probes conjugated with these 10 fluorophores were used in UltraPCR experiments for fluorophore characterization.

Photobleaching scans were performed on FAM vs. Alexa 488, Bodipy TMR-X vs. HEX, and Cy5 vs. Atto 647N samples in corresponding channels to confirm that FAM, Bodipy TMR-X, and Cy5 were photobleaching sensitive, while Alexa 488, HEX, and Atto 647N were photobleaching resistant (defined as minimal decrease in fluorescence intensity after up to 7 repeated scans at 480 ms per scan per cross section). Dy-485XL, Dy-520XL, and TAMRA were further scanned using the same excitation lasers as FAM/Alexa 488 or Bodipy TMR-X/HEX but different emission filters to confirm that they were separable from other fluorophores. In the end, a 10-scan approach with 7 scans before photobleaching and 3 scans after photobleaching for 480 ms was used for characterizing the 10 fluorophores and profiling each fluorophore's distinct signature among our 10 channels. This setting was then programmed into UltraPCR Imager for all automated sample imaging.

10-color TaqMan multiplexing: To show compatibility of 10 different fluorescent dyes in the 4-color system, a 10-plex panel was designed to include targets from the human mRNA transcripts (CCL3L1, PLGA1, ATF4, GAPDH, CD7), N1 region from Sars-CoV-2, genomic loci on human chromosomes 13 and 18, synthetic template, and gene prfA from L. cytomonogenes. Each target was labeled with 1 of the 10 fluorescent dyes conjugated to hydrolysis probes. Each stock tube of synthetic template of each target was quantified by UltraPCR to obtain copies/μL. Next, all 10 synthetic templates, forward primers, reverse primers, and probes were assayed in one tube in duplicates (10-plex). Separately, individual synthetic templates with their respective forward primers, reverse primers, and probes were assayed in duplicate (1-plex). After emulsion generation and thermal cycling, image analysis was performed where positive partitions were counted and characterized by fluorescence level in 10 channels. The dimensional reduction software package UMAP was used to reduce the 10 channels to clusters along an x-y axis. The clusters were used to generate counts and 1-plex counts were compared to their respective clusters in the 10-plex samples.

Comboplex Approaches: For comboplex with hydrolysis probes, probes targeting the same sequence with different fluorescent dyes were used so that each target may be labeled with multiple colors. Synthetic template for prfA, primers and 1 to 3 hydrolysis probes were added in the mixture and followed hydrolysis probe amplifying condition. For the 22-plex comboplex experiments, UltraPCR's Universal Multiplex (UM) Kit and UltraPCR Mix were used to perform multiplexing without the use of hydrolysis probes. UM technology utilizes a 5′ adapter to a PCR forward primer to label targets without the use of hydrolysis probes. Targets were labeled with either 1 color or 2 colors using different combinations of forward primers. For 1-color labeling, 1 forward primer per target was used and for 2-colors labeling, 2 forward primers per target were used. Once minimal nonspecific interactions between all primers and probes were confirmed without template addition, all the primers and probes for the 22 targets were pooled together. Once pooled, the primers were filtered (0.1 μm) for 2 minutes at 12,000×g. Final primer concentration for targets labeled with 2 colors were 20 nM each for the two forward primers and 200 nM for the reverse primer. For targets labeled with 1 color, the final concentrations were 40 nM and 200 nM for the forward and reverse primer, respectively. Each synthetic target template was added accordingly depending on 4 plex, 8 plex and 22 plex conditions. Annealing temperature in UM thermal cycling condition was increased from 56° C. to 60° C. to minimize non-specific amplifications.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method comprising:
(a) providing a plurality of partitions comprising at least 1,000 partitions, wherein each partition of said plurality of partitions comprises:
(i) a portion of a nucleic acid sample; and
(ii) processing materials,
wherein a distribution of nucleic acid material of said nucleic acid sample to partition has an average of at least 10 femtograms of said nucleic acid material per partition and wherein each partition comprises an average volume of said plurality of partitions is less than or equal to 10 picolitre (pl);
(b) reacting said processing materials of each partition of said plurality of partitions with said portion of said nucleic acid sample of each partition of said plurality of partitions;
(c) detecting signals from at least a subset of said plurality of partitions; and
(d) using said signals detected in (c) to identify a plurality of nucleic acid molecules of said nucleic acid sample.

2. The method of claim 1, wherein said plurality of partitions comprises at least 10,000 partitions.

3. The method of claim 1, wherein said plurality of partitions comprises at least 1,000,000 partitions.

4. The method of claim 1, wherein said distribution of nucleic acid material to partition has an average of at least 100 femtograms of said nucleic acid sample per partition.

5. The method of claim 1, wherein said distribution of nucleic acid material to partition has an average of at least 200 femtograms of said nucleic acid sample per partition.

6. The method of claim 1, wherein said average volume of said plurality of partitions is less than or equal to 5 pl.

7. The method of claim 1, wherein said average volume of said plurality of partitions is less than or equal to 1 pl.

8. The method of claim 1, wherein said nucleic acid sample comprises at least 0.1 microgram of nucleic acid material.

9. The method of claim 1, wherein said nucleic acid sample comprises at least 0.5 microgram of nucleic acid material.

10. The method of claim 1, wherein said nucleic acid sample comprises at least 1.0 microgram of nucleic acid material.

11. The method of claim 1, wherein said plurality of partitions are part of a gel matrix.

12. The method of claim 1, wherein said plurality of partitions are part of an emulsion.

13. The method of claim 1, wherein said nucleic acid sample comprise cell-free nucleic acid molecules.

14. The method of claim 13, wherein said cell-free nucleic acid molecules comprise deoxyribonucleic acid molecules.

15. The method of claim 13, wherein said cell-free nucleic acid molecules comprise ribonucleic acid molecules.

16. A method comprising:
(a) providing a plurality of partitions comprising at least 1,000,000 partitions, wherein each partition of said plurality of partitions comprises:
(i) a portion of a nucleic acid sample; and
(ii) processing materials,
wherein a distribution of nucleic acid material of said nucleic acid sample to partition has an average of at least 10 femtograms of said nucleic acid material per partition;
(b) reacting said processing materials of each partition of said plurality of partitions with said portion of said nucleic acid sample of each partition of said plurality of partitions;
(c) detecting signals from at least a subset of said plurality of partitions; and
(d) using said signals detected in (c) to identify a plurality of nucleic acid molecules of said nucleic acid sample.

17. The method of claim 16, wherein said plurality of partitions comprises at least 10,000,000 partitions.

18. The method of claim 17, wherein said plurality of partitions comprises at least 20,000,000 partitions.

19. The method of claim 16, wherein said distribution of nucleic acid material to partition has an average of at least 100 femtograms of said nucleic acid sample per partition.

20. The method of claim 16, wherein said distribution of nucleic acid material to partition has an average of at least 200 femtograms of said nucleic acid sample per partition.
